(19) United States
(12) Patent Application Publication
(10) Pub. No.: US 2013/0045181 A1
(43) Pub. Date: Feb. 21, 2013

(54) Methods and Compositions for Chlamydial Antigens for Diagnosis and Treatment of Chlamydial Infection and Disease

(71) Applicant: Guangming Zhong, San Antonio, TX (US)

(72) Inventor: Guangming Zhong, San Antonio, TX (US)

(21) Appl. No.: 13/583,475

(22) PCT Filed: Mar. 9, 2011
(86) PCT No.: PCT/US11/27678

§ 371 Date: Nov. 8, 2012

Related U.S. Application Data
(60) Provisional application No. 61/312,000, filed on Mar. 9, 2010.

(51) Int. Cl. A61K 39/118; C40B 30/04; A61P 15/00; A61K 39/39; A61P 31/04; G01N 33/571; C07K 14/295

(52) U.S. Cl. 424/85.2; 436/501; 435/7.1; 506/9; 435/7.92; 530/350; 424/263.1

(57) ABSTRACT

The present invention provides Chlamydia proteins and methods of use in treatment and immunization protocols as well as in diagnostic and detection assays.

### STATEMENT OF PRIORITY

This application claims the benefit, under 35 U.S.C. §119(e), of U.S. Provisional Application Ser. No. 61/312,000, filed Mar. 9, 2010, the entire contents of which are incorporated by reference herein.

### STATEMENT OF GOVERNMENT SUPPORT

Aspects of the present invention were funded by government support under grant number AI47997 from the National Institutes of Health. The United States Government has certain rights in this invention.

### BACKGROUND OF THE INVENTION

1. Field of the Invention

The present invention relates to the field of diagnosis of chlamydial infection and disease as well as treatment/prevention of chlamydial infection and disease.

2. Background Art

Infection with Chlamydia trachomatis, a species of obligate intracellular bacterial pathogens, can cause various diseases in humans depending on the site of infection. Ocular infection can lead to blinding trachoma (1), while infection in the urogenital tract is a leading cause of sexually transmitted bacterial diseases (2). Although chlamydial infection is susceptible to antibiotic treatment, many urogenitally infected individuals don't seek treatment due to lack of obvious clinic symptoms, thus becoming vulnerable to persistent infection and the development of complications such as ectopic pregnancy and infertility (3,4). Vaccination is considered the most effective means for preventing chlamydial infection and diseases. However, inactivated whole organism-based vaccines failed in human trachoma trials (5). Despite the extensive efforts in developing subunit vaccines in past decades, there is still no licensed C. trachomatis vaccine, likely due to insufficient knowledge of protective determinants of chlamydial organisms.

Chlamydia-induced pathologies are largely due to inflammation triggered by chlamydial and host factors released during intracellular replication and lysis of the infected cells (6,7). Thus, chlamydial intracellular replication is considered a major contributor to chlamydial pathogenesis (8,9).

A typical intracellular infection starts with C. trachomatis entry into epithelial cells in the form of an elementary body (EB) that is infectious but metabolically inert (10,11). The EB-laden vacuole not only resists fusion with lysosomes but also supports chlamydial replication. The intravacuolar EB can rapidly differentiate into reticulate bodies (RBs) that are metabolically active but non-infectious. After replication within cytoplasmic vacuoles (also termed inclusions), the progeny RBs can differentiate back into EBs for spreading to adjacent cells.

To ensure successful completion of intracellular growth, chlamydial organisms have evolved multiple strategies for overcoming the host defense mechanisms encountered during the growth cycle. One of the chlamydial strategies is to secrete proteins outside of the chlamydial organisms for modifying intra-inclusion lumenal environments (12), decorating the inclusion membrane (13,14,15,16) and/or manipulating host signaling pathways in the host cell cytosol (17).

Some of the secreted proteins are preexisting proteins associated with the infectious EBs (18), while others are newly made during infection (17). Not all proteins newly synthesized during infection are packaged into the infectious EBs. For example, a chlamydial protease designated as CPAF is secreted into the host cell cytosol and can be detected only in the infected cell culture but not in the purified EB organisms (17). These types of proteins are defined herein as infection-dependent or in vivo expressed proteins. These proteins are produced and significantly enriched during live infection but only minimally packaged into the infectious organisms. It is very difficult to directly detect chlamydial proteins during natural infection in humans. Fortunately, antigen-specific antibodies can be used to indirectly monitor the expression of the infection-dependent antigens in humans (19).

The determination that inactivated whole chlamydial organism-based vaccines failed to induce protective immunity in humans (5) and that live chlamydial organism infection always induces better protective immunity than immunization with inactivated organisms in animal models suggests that some infection-dependent antigens may be able to induce protective responses. This is supported by the observation that CPAF, an infection-dependent antigen, has been found to induce protective immunity in mice (20). It is thus important to identify the infection-dependent antigens (that are expressed during chlamydial infection in humans) and to determine their roles in protective immunity.

The present invention overcomes previous shortcomings in the art by providing chlamydial antigens that can be used to develop rapid and convenient means for diagnosing chlamydial invention and to design effective treatment protocols and vaccines for treating and preventing chlamydial infection and diseases.

### SUMMARY OF THE INVENTION

The present invention provides a composition comprising one or more than one isolated Chlamydia trachomatis protein selected from the group consisting of (1) pCT03, (2) CT858, (3) CT841, (4) CT443, (5) CT143, (6) CT101, (7) CT694, (8) CT813, (9) CT142, (10) CT089, (11) CT442, (12) CT529, (13) CT806, (14) CT147, (15) CT119, (16) CT240, (17) CT812, (18) CT798, (19) CT067, (20) CT695, (21) CT875, (22) CT681, (23) CT795, (24) CT022, (25) CT456, (26) CT828, (27) CT381, an immunogenic fragment thereof, a homologue thereof from a different Chlamydia species and any combination thereof.

In a specific embodiment, the present invention provides a composition comprising one or more than one isolated Chlamydia trachomatis protein selected from the group consisting of pCT03, CT858, CT443, CT143, CT813, CT119, CT240, CT695, CT828, CT381, an immunogenic fragment thereof, a homologue thereof from a different Chlamydia species and any combination thereof.

In some embodiments, the composition of this invention can comprise one or more than one isolated Chlamydia trachomatis protein selected from the group consisting of (1) pCT03, (2) CT858, (4) CT443, (7), CT694, (10) CT089, (11) CT 442, (12) CT529, (14) CT147, (15) CT119, (17) CT812, (20) CT695, (22) CT681, (23) CT795, an immunogenic fragment thereof, a homologue thereof from a different Chlamydia species and any combination thereof.

In various embodiments, the compositions of this invention can further comprise an isolated Chlamydia trachomatis protein selected from the group consisting of porin B (PorB), CT110 (HSP90), CT681 (MOMP), an immunogenic fragment thereof, a homologue thereof from a different Chlamydia species and any combination thereof.

The compositions of this invention can be in a pharmaceutically acceptable carrier and in some embodiments can further comprise an adjuvant and/or an immunostimulant, which can be, but is not limited to, CpG, IL-12 and any combination thereof.

The present invention further provides embodiments wherein the compositions of this invention can also comprise a protein or immunogenic fragment thereof of a pathogenic organism other than Chlamydia trachomatis. Such pathogenic organism can be, but is not limited to Chlamydia muridarum, Chlamydia pneumoniae, Chlamydia caviae. Trichomonas vaginalis, Candida albicans, Neisseria gonorrheae, Treponema pallidum, herpes simplex virus, human papilloma virus and human immunodeficiency virus and any combination thereof.

Additional aspects of this invention include a method of eliciting an immune response against Chlamydia in a subject, comprising administering to the subject an effective amount of a composition of this invention, thereby eliciting an immune response against Chlamydia in the subject.

Furthermore, the present invention provides a method of inducing immunity against Chlamydia in a subject, comprising administering to the subject an amount of a composition of this invention sufficient to elicit an immune response, wherein said immune response is sufficient to decrease risk of onset of disease caused by Chlamydia.

In addition, the present invention provides a method of eliciting an immune response against Chlamydia in a subject, comprising administering to the subject an effective amount of a composition of this invention comprising a combination of chlamydial proteins or immunogenic fragments thereof of this invention, wherein said combination has a synergistic effect in eliciting an immune response, thereby eliciting an immune response against Chlamydia in the subject.

Also provided herein is a method of treating an infection by Chlamydia in a subject, comprising administering to the subject an effective amount of a composition of this invention, thereby treating an infection by Chlamydia in the subject.

In addition, the present invention provides a method of preventing infection by Chlamydia in a subject, comprising administering to the subject an effective amount of a composition of this invention, thereby preventing infection by Chlamydia in the subject.

The present invention further provides a method of reducing the likelihood of infertility due to Chlamydia infection in a subject, comprising administering to the subject an effective amount of a composition of this invention, thereby reducing the likelihood of infertility due to Chlamydia infection in the subject.

Further provided herein is a method of reducing the incidence of hydrosalpinx due to Chlamydia infection in a subject, comprising administering to the subject an effective amount of a composition of this invention, thereby reducing the incidence of hydrosalpinx due to Chlamydia infection in the subject.

The present invention additionally provides a method of detecting an antibody to Chlamydia in a sample, comprising: a) contacting the sample with a composition of this invention under conditions whereby an antigen/antibody complex can form; and b) detecting antigen/antibody complex formation, thereby detecting an antibody to Chlamydia in the sample.

Also provided herein is a method of diagnosing a Chlamydia infection in a subject, comprising: a) contacting a sample from the subject with a composition of this invention under conditions whereby an antigen/antibody complex can form; and b) detecting antigen/antibody complex formation, thereby diagnosing a Chlamydia infection in the subject.

The present invention further provides a method of detecting a Chlamydia protein in a sample, comprising: a) contacting the sample with an antibody that specifically binds a Chlamydia protein or immunogenic fragment thereof of this invention under conditions whereby an antigen/antibody complex can form; and b) detecting antigen/antibody complex formation, thereby detecting a Chlamydia protein in the sample.

In addition, a method is provided of diagnosing a Chlamydia infection in a subject, comprising: a) contacting a sample from the subject with an antibody that specifically binds a Chlamydia protein or immunogenic fragment thereof of this invention under conditions whereby an antigen/antibody complex can form; and b) detecting antigen/antibody complex formation, thereby diagnosing a Chlamydia infection in the subject.

### BRIEF DESCRIPTION OF THE DRAWINGS

FIG. 1. Reactivity of 99 STI antisera with 934 GST-chlamydial fusion proteins. Each of the 99 human antibodies (displayed along the X-axis) after 1:500 dilution was reacted with each of the 934 GST fusion proteins (listed along the Y-axis) immobilized onto the 96 well microplates. The human antibody binding was detected with a secondary goat anti-human IgG antibody conjugated with HRP plus a soluble substrate. The results were expressed as OD readings obtained at the wavelength of 405 nm. Any given reaction with an OD reading 4 fold above the value from the control well (GST alone-coated well) in the same microplate was determined positive. The positive OD values were expressed as binding intensity in increasing brightness of green fluorescent color while the negative OD values were always in black as indicated at the bottom of the figure. The 934 fusion proteins representing 909 unique ORFs encoded by C. trachomatis genome and plasmid were listed first in order of the ORFs from CT001 to CT875 plus 8 DEGs and 8 pCTs (left panel). Both letter and number extensions were used to distinguish multiple fusion proteins and protein fragments that share the same ORF names. The 934 fusion proteins were then reordered based on frequency recognized by the 99 human antisera (middle panel) as indicated along the left side of the panel. Both frequencies and numbers of antigens recognized (in brackets) at a given frequency were marked. The 719 antigens recognized by at least one antiserum were marked as the B cell ANTIGENome of C. trachomatis and the remaining 190 fusion proteins not recognized by any human antisera were marked along the right side of the panel. The 27 proteins recognized by 50% or more human antisera were designed as immunodominant antigens as indicated in the right panel. The immunodominant antigens cover a wide array of chlamydial proteins, some of which were identified as immunodominant antigens for the first time in the current study.

FIGS. 2A-C. Recognition of the 934 fusion proteins by one of the 99 human antisera in microplate ELISA. Each of the 99 human serum samples was reacted with the fusion proteins arrayed onto the microplates after the serum sample was pre-absorbed with GST-containing bacterial lysates (A) or further absorption with either Chlamydia-infected cells lysates (B) or HeLa alone lysates (C). The human antibody binding was visualized with a goat anti-human IgG conjugated with HRP and ABTS substrate (green). Only the remaining antibody binding that was still positive compared to the control well in the sample plate after HeLa alone lysate absorption but significantly reduced after Chlamydia-infected cell lysate absorption was counted as positive reactivity. Arrows indicate examples of three positive wells. The positive and negative control wells in each plate were marked with boxes as shown and the blank wells in the last plate were also marked with a box as shown. Binding of each human antiserum to the fusion proteins was confirmed in this way. For this particular antiserum, 54 fusion proteins, each representing a unique ORF, were determined to be positive antigens.

FIGS. 3A-B. Western blot detection of 54 chlamydial fusion proteins and precipitation of endogenous chlamydial proteins by the human antibodies. (A) The 54 GST fusion proteins were loaded onto a SDS gel and after electrophoresis separation, one set of the gels were subjected to Coomassie Blue dye staining for visualizing the total proteins. The parallel gels were used in a Western blot for measuring the human antibody reactivity. The same human positive serum used in FIG. 2 was 10 fold serially diluted and reacted with the Western blot membrane. At 1:1000 dilution, many GST fusion proteins were picked up by the human antibodies (panel b). As the dilution increased to 1:10,000, fewer proteins were recognized (c) and only a few proteins were detectable when the serum was diluted another 10 fold (d). No significant reactivity was detected when a negative serum was measured at 1:1000 dilution (e). Among the 54 GST fusion proteins, only 44 were reasonably recognized by the human serum while the remaining 10 fusion proteins were not or only minimally detected by the antiserum even at 1:1,000 (panel b, marked with white stars). (B) Lysates of HeLa cells infected with C. trachomatis D organisms were precipitated with the same positive and negative human antisera described in FIG. 2 but conjugated to protein A/G agarose beads, respectively. The HeLa alone lysates, Chlamydia-infected HeLa lysates and the human antibody-specific precipitates were resolved in a SDS polyacrylamide gel. The gel-separated protein bands were blotted onto a nitrocellulose membrane for Western blot detection of chlamydial proteins including CT341 (negative control, panel a), MOMP (positive control, b), CT101 (c), CT208 (d), CT222 (e), CT403 (f), CT437 (g), CT542 (h), CT610 (i), CT771 (j), CT798 (k), and pCT03 (Pgp3, 1) with mouse antibodies raised against the corresponding chlamydial fusion proteins. The mouse antibody binding was visualized with a goat-mouse IgG conjugated with HRP with standard ECL. Positive but not negative antiserum precipitated the endogenous chamydial proteins from Chlamydia-infected cell lysates, which is consistent with the proteome array ELISA result shown in FIG. 2.

FIG. 4. Human antibody recognition of 27 immunodominant chlamydial proteins in Western blot. The 27 chlamydial GST fusion proteins were detected by human antisera pooled from 99 patients on Western blot as described in FIG. 3A. Among the 27 GST fusion proteins, only 17 were reasonably recognized by the human antibodies while the remaining 10 fusion proteins were not or only minimally detected by the pooled human antisera.

FIG. 5. Comparison of C. trachomatis antigen profiles recognized by human, rabbit and mouse antibodies. The 934 GST fusion proteins (listed along the Y-axis) immobilized onto the 96 well microplates were reacted with each of the antisera from 99 women (as described in FIG. 1), 7 Balb/c (B/c) mice intranasally infected (in), 18 Balb/c mice intravaginally infected (iv), 12 C57BL mice (C57) intravaginally infected with live chlamydial serovar D organisms and 5 Balb/c mice intraperitoneally (ip) and 13 rabbits intramuscularly (im) immunized with dead chlamydial serovar D organisms as indicated on top of the figure. The individual antibody binding intensity was expressed as increasing brightness of the corresponding colors as described in FIG. 1. The 934 chlamydial fusion proteins were first listed in order of the ORFs (left panel) and then resorted based on the frequency of recognition by human antibodies (right panel). Although many antigens frequently recognized by human antisera were also dominantly recognized by animal antisera (marked with vertical bars along the right side of the right panel), some human-recognized antigens were not detected by animals at all, especially the immunized animals (marked with arrows). There were also antigens that were dominantly recognized by immunized animals that were only minimally or not recognized by human antibodies (arrowheads).

FIG. 6. Comparison of C. trachomatis antigen profiles recognized by different strains of mice infected or immunized via different routes. The antigen profiles recognized by antisera from Balb/c or C57BL mice infected or immunized with live or dead C. trachomatis serovar D organisms as described in FIG. 5 were compared. The 934 fusion proteins were first listed along the Y-axis in the order of ORFs (panel A) and then reordered based on mouse antibody recognition frequencies (panel B). All antigens detected by mouse antibodies were re-emphasized in panel C. Proteins that were recognized by live organism-infected mice were defined as infection-dependent antigens while those detected by both infected and immunized or immunized alone were defined as infection-independent antigens as marked along the right side of the figure. The total number of antigens recognized by one or more mice in each group was listed at the bottom of panel C.

FIG. 7. Recognition of infection-dependent and -independent antigens by infected and immunized host species. The C. trachomatis antigen profiles recognized by 6 groups of antisera were acquired as described in FIG. 5. The 934 GST fusion proteins (listed along the Y-axis) were resorted based on the recognition by both immunized animals and infected humans (left panel). Proteins recognized by infected humans or animals but not immunized animals were defined as infection-dependent antigens, otherwise as infection-independent antigens as indicated along the left side. The antigens from each group with top 40% recognition frequency by human antisera were re-emphasized in the right panel and the corresponding antigen ORF numbers were listed along the left side. The ORF numbers in bold indicate that the corresponding antigens were analyzed in FIG. 8.

FIG. 8. Detection of infection-dependent and -independent antigens in Chlamydia-infected cells versus purified organisms. HeLa cells with (HeLa-D) or without (HeLa) infection with C. trachomatis and purified EBs or RBs were resolved in a SDS polyacrylamide gel. The gel-separated protein bands were blotted onto a nitrocellulose membrane for Western blot detection of chlamydial proteins including representative antigens from the infection-dependent group such as CPAF (panel a), CT694 (b), CT813 (c), CT529 (d), CT806 (e) & CT828 (f) and representative antigens from the infection-independent group such as pCT03 (Pgp3, g), CT443 (h), CT143 (i), CT067 (j), CT681 (k), CT022 (1), CT456 (m) and CT823 (n). Most of the infection-dependent antigens were dominantly detected in the infected cell samples but with either reduced (CT694 & CT806) or undetectable (CT858, CT813 & CT828) amounts in the purified EB organisms. In comparison, the infection-independent antigens were always detected in the purified EB organisms.

FIG. 9. IFU of CT694(TC0066) in Vac1.

FIG. 10. IFU of TC0726(CT442).

FIG. 11. IFU of TC0177(CT795) in Vac3.

FIG. 12. IFU of TC0067(CT695) in VacExp4.

FIG. 13. IFU of TC0396 (CTI 19) and TC0052 (CT681) in Vac5.

FIG. 14. IFU of TC0727 (CT443).

FIG. 15. IFU of TC0364 (CT089) in Vac7.

FIG. 16. IFU of TC0197M(CT812) in VacB.

FIG. 17. pMoPN03(pCT03) IFU shedding in VacExp4.

FIG. 18. pMoPn03(PCT03) IFU shedding in VacExp7.

FIG. 19. IFU of CT858 in Vac2 (Balb/c, IN).

FIG. 20. IFU of CT858 in Vac5 (Balb/c, IM).

FIG. 21. IFU of TC0376, TC0190, TC0229 and TC0419 in Vac1.

FIG. 22. IFU of TC0420(CT143) in Vac1.

FIG. 23. IFU of TC0420(CT143) in Vac3.

FIG. 24. IFU of TC0199(CT813) in VacA.

FIG. 25. IFU of TC0511(CT240) in Vac2.

FIG. 26. IFU of TC0511(CT240) in Vac3.

FIG. 27. IFU or TC0338 and TC0181 in Vac7.

FIG. 28. IFU of TC0291(CT022) in Vac5.

FIG. 29. IFU of TC0741(CT456) in Vac2.

FIG. 30. IFU of CT828 in Vac2.

FIG. 31. IFU of TC0215(CT828) in Vac7.

FIG. 32. IFU of TC0660(CT381) in Vac6.

### DETAILED DESCRIPTION OF THE INVENTION

The present invention is based on the discovery of immunodominant proteins of Chlamydia trachomatis and particularly the identification of chlamydial proteins that are “infection-dependent” or “in vivo expressed antigens.” These immunodominant proteins have been identified by the screening fusion protein arrays described in the EXAMPLES section herein. These chlamydial proteins and immunogenic fragments thereof can be employed in methods of treating infection and disease caused by Chlamydia as well as in methods of prophylaxis (e.g., as a vaccine) to prevent or delay onset of infection and disease caused by Chlamydia. Furthermore, proteins, immunogenic fragments thereof and/or homologues thereof from other chlamydial species can be employed in methods of treating infection and disease caused by Chlamydia as well as in methods of prophylaxis (e.g., as a vaccine) to prevent or delay onset of infection and disease caused by Chlamydia. These immunodominant proteins, immunogenic fragments thereof and/or homologues of these proteins or immunogenic fragments from other chlamydial species can also be employed in methods of detection and diagnosis by identifying the presence of the chlamydial protein or the presence of an antibody to the chlamydial protein in a sample, such as a biological sample from a subject.

Thus, the present invention provides a composition comprising, consisting essentially of, or consisting of one or more than one (e.g., 2, 3, 4, 5, 6, 7, 8, 9, 10, 11, 12, 13, 14, 15, 16, 17, 18, 19, 20, 21, 22, 23, 24, 25, 26 or 27) isolated Chlamydia trachomatis protein selected from the group consisting of (1) pCT03, (2) CT858, (3) CT841, (4) CT443, (5) CT143, (6) CT101, (7) CT694, (8) CT813, (9) CT142, (10) CT089, (11) CT442, (12) CT529, (13) CT806, (14) CT147, (15) CT119, (16) CT240, (17) CT812, (18) CT798, (19) CT067, (20) CT695, (21) CT875, (22) CT681, (23) CT795, (24) CT022, (25) CT456, (26) CT828, (27) CT381, an immunogenic fragment thereof, a homologue thereof from a different Chlamydia species and any combination thereof.

Furthermore, the present invention provides a composition comprising, consisting essentially of or consisting of one or more than one (e.g., 2, 3, 4, 5, 6, 7, 8, 9, 10 or 11) isolated Chlamydia trachomatis protein selected from the group consisting of (1) pCT03, (2) CT858, (4) CT443, (7), CT694, (10) CT089, (11) CT 442, (12) CT529, (14) CT147, (15) CT119, (17) CT812, (20) CT695, (22) CT681, (23) CT795, an immunogenic fragment thereof, a homologue thereof from a different Chlamydia species and any combination thereof.

In a specific embodiment, the present invention provides a composition comprising, consisting essentially of and/or consisting of one or more than one (e.g., 2, 3, 4, 5, 6, 7, 8, 9, or 10) isolated Chlamydia trachomatis protein selected from the group consisting of pCT03, CT858, CT443, CT143, CT813, CT119, CT240, CT695, CT828, CT381, an immunogenic fragment thereof, a homologue thereof from a different Chlamydia species and any combination thereof.

In additional embodiments, the compositions of this invention can further comprise, consist essentially of, or consist of an isolated Chlamydia trachomatis protein selected from the group consisting of porin B (PorB) (e.g., as described in USPTO Publication No. 2005/0266016, the entire contents of which are incorporated by reference herein), CT110 (HSP90), CT858 (CPAF), CT681 (MOMP), an immunogenic fragment thereof, a homologue thereof from a different Chlamydia species and any combination thereof.

In yet further embodiments, any of the compositions of this invention can comprise, consist essentially of, or consist of one or more than one (e.g., 2, 3, 4, 5, 6, 7, 8, 9, 10, etc.) isolated Chlamydia trachomatis protein as set forth in International Application Serial No. PCT/US09/05541, filed Oct. 9, 2009 and entitled “Methods and compositions for chlamydial antigens for diagnosis and treatment of chlamydial infection and disease,” the entire contents of which are incorporated by reference herein. The one or more Chlamydia trachomatis protein as set forth in International Application Serial No. PCT/US09/05541 can be selected from any of proteins 1-156 of Table 1 of International Application Serial No. PCT/US09/05541, any of proteins 1-19 of Table 2 International Application Serial No. PCT/US09/05541, any of proteins 1-41 of Table 3 International Application Serial No. PCT/US09/05541, an immunogenic fragment thereof; a homologue thereof from a different Chlamydia species and any combination thereof.

In addition, the present invention provides a pharmaceutical composition comprising, consisting essentially of, or consisting of an isolated Chlamydia trachomatis protein of this invention, an immunogenic fragment thereof and/or a homologue thereof from a different Chlamydia species in a pharmaceutically acceptable carrier. These proteins, immunogenic fragments and homologues can be present in a composition of this invention in any combination and in any ratio relative to one another.

In certain embodiments, the fragments and/or polypeptides of this invention can be fused with a “carrier” protein or peptide to produce a fusion protein. For example, the carrier protein or peptide can be fused to a polypeptide and/or fragment of this invention to increase the stability thereof (e.g., decrease the turnover rate) in the cell and/or subject. Exemplary carrier proteins include, but are not limited to, glutathione-S-transferase or maltose-binding protein. The carrier protein or peptide can alternatively be a reporter protein. For example, the fusion protein can comprise a polypeptide and/or fragment of this invention and a reporter protein or peptide (e.g., green fluorescent protein (GFP), β-glucoronidase, β-galactosidase, luciferase, and the like) for easy detection. As a further alternative, the fusion protein attached to the polypeptides and/or fragments and a carrier protein or peptide can be targeted to a subcellular compartment of interest, i.e., to affect the co-localization of the polypeptide and/or fragment. Any suitable carrier protein as is well known in the art can be used to produce a fusion protein of this invention.

The present invention further provides an isolated nucleic acid encoding a Chlamydia protein or immunogenic fragment thereof or homologue thereof of this invention, the nucleotide sequences of which are well known in the art. Such nucleic acids can be present in a vector (e.g., a viral vector such as vaccinia virus, adenovirus, adeno-associated virus, lentivirus, herpes virus, alphavirus vectors, etc., as are well known in the art), which can be present in a cell (e.g., a cell transformed by the introduction of heterologous nucleic acid).

In additional embodiments of this invention, the chlamydial proteins, immunogenic fragments and/or homologues of this invention can be employed in the methods and compositions of this invention either singly, in multiples of the same and/or different proteins or immunogenic fragments and/or in any combination (e.g., 2, 3, 4, 5, 6, 7, 8, 9, 10 or more) with one another and/or in any combination with any other chlamydial protein, non-chlamydial protein and/or reagent of this invention.

The present invention further includes isolated polypeptides, peptides, proteins and/or fragments that are substantially equivalent to those described for this invention. As used herein, “substantially equivalent” can refer both to nucleic acid and amino acid sequences, for example a mutant sequence, that varies from a reference sequence by one or more substitutions (e.g., substitution with conservative amino acids as are well known in the art), deletions and/or additions, the net effect of which does not result in an undesirable adverse functional dissimilarity between reference and subject sequences. In some embodiments, this invention can include substantially equivalent sequences that have an adverse functional dissimilarity. For purposes of the present invention, sequences having equivalent biological activity and equivalent expression characteristics are considered substantially equivalent.

The invention further provides homologues, as well as methods of obtaining homologues, of the polypeptides and/or fragments of this invention from other strains of Chlamydia and/or other organisms included in this invention. As used herein, an amino acid sequence or protein is defined as a homologue of a polypeptide or fragment of the present invention if it shares significant homology or identity with one of the polypeptides and/or fragments of the present invention. Significant homology or identity means at least 50%, 55%, 60%, 65%, 70%, 75%, 80%, 85%, 90%, 95%, 98% and/or 100% homology or identity with another amino acid sequence. Such homologues can also be identified by having significant identity at the nucleotide sequence level (i.e., at least 50%, 55%, 60%, 65%, 70%, 75%, 80%, 85%, 90%, 95%, 98% and/or 100% or identity with another nucleotide sequence).

As one example, by using the nucleic acids that encode the chlamydial proteins or fragments of this invention (as are known in the art and incorporated by reference herein), as a probe or primer, and techniques such as PCR amplification and colony/plaque hybridization, one skilled in the art can identify homologues of the polypeptides and/or fragments of this invention in Chlamydia and/or other organisms on the basis of information available in the art.

As another example, homologues of the chlamydial proteins of this invention can be identified by having cross-reactivity when analyzed in immunoassays employing antibodies that specifically react with a chlamydial protein or immunogenic fragment of this invention.

As one non-limiting example, a listing of Chlamydia pneumoniae proteins and the Chlamydia trachomatis homologues of these proteins can be found in U.S. Pat. No. 6,822,071, the entire contents of which are incorporated by reference herein for these teachings.

The present invention further provides isolated nucleic acids, vectors and cells of this invention for use in the methods described herein. Thus, in particular embodiments, a nucleic acid of this invention encoding a Chlamydia protein, an immunogenic fragment and/or a homologue of this invention can be introduced into a subject under conditions well known in the art, wherein the nucleic acid is expressed and the encoded product is produced to elicit an immune response in the subject, thereby treating and/or preventing a Chlamydia infection and/or disease. Thus, the nucleic acids, vectors and/or cells of this invention can be present in a composition comprising a pharmaceutically acceptable carrier. The nucleic acids of this invention can also be used in detection and diagnostic assays according to the methods described herein and as are well known in the art.

In additional embodiments of this invention, the compositions of this invention can comprise a protein and/or immunogenic fragment thereof of a different pathogenic organism in any combination [e.g., a pathogenic organism that is sexually transmitted, including but not limited to: Trichomonas (e.g., Trichomonas vaginalis); a pathogenic yeast or fungus (e.g., Candida albicans), Neisseria (e.g., N. gonorrhea), Treponema pallidum, and pathogenic viruses (e.g., herpes simplex virus (HSV), human immunodeficiency virus (HIV), human papilloma virus (HPV)]. The compositions of the present invention can also comprise a protein and/or immunogenic fragment from other chlamydial species, including but not limited to Chlamydia muridarum, Chlamydia pneumoniae and Chlamydia caviae.

### DEFINITIONS

As used herein, “a,” “an” or “the” can mean one or more than one (e.g., 2, 3, 4, 5, 6, 7, 8, 9, 10, etc.). For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, the term “consists essentially of” (and grammatical variants) means that the immunogenic composition of this invention comprises no other material immunogenic agent other than the indicated agent(s). The term “consists essentially of” does not exclude the presence of other components such as adjuvants, immunomodulators, and the like.

As used herein, by “pharmaceutically acceptable” is meant a material that is not biologically or otherwise undesirable, i.e., the material may be administered to a subject without causing appreciable or undue undesirable or adverse biological effects. Thus, such a pharmaceutical composition may be used, for example, to prepare compositions for

immunization. Physiologically and pharmaceutically acceptable carriers may contain other compounds including but not limited to stabilizers, salts, buffers, adjuvants and/or preservatives (e.g., antibacterial, antifungal and antiviral agents) as are known in the art. The pharmaceutically acceptable carrier can be sterile in some embodiments and/or formulated for delivery into and/or administration to a subject of this invention.

A “pharmaceutically acceptable component” such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include, without limitation, any of the standard pharmaceutical carriers such as phosphate buffered saline solutions, water, emulsions such as oil/water emulsion, microemulsions and various types of wetting agents.

The terms “epitope,” “antigenic epitope” or “antigenic peptide” as used herein refers to at least about 3 to 5, or about 5 to about 10 or about 5 to about 15, and not more than about 1,000 amino acids (or any integer therebetween), which define a sequence that by itself or as part of a larger sequence, binds to an antibody generated in response to such sequence or stimulates a cellular immune response. Thus, the epitope, when present in the proper conformation, provides a reactive site for an antibody or T cell receptor. The identification of epitopes can be carried out by immunology protocols that are well known in the art. There is no critical upper limit to the length of the fragment, which can comprise nearly the full-length of the protein sequence, or even a fusion protein comprising two or more epitopes from a single or multiple chlamydial proteins. An epitope for use in the subject invention is not limited to a polypeptide having the exact sequence of the portion of the parent protein from which it is derived. Indeed, there are many known strains or isolates of Chlamydia and there are several variable domains that exhibit relatively high degrees of variability between isolates. Thus, the term “epitope” encompasses sequences identical to the native sequence, as well as modifications to the native sequence, such as deletions, additions and substitutions (generally, but not always, conservative in nature).

As set forth herein, the term “immunogenic fragment” means a fragment (e.g., a peptide) of a protein that can stimulate either humoral or cellular immune responses in the subject. An immunogenic fragment of this invention can comprise, consist essentially of and/or consist of one, two, three, four or more epitopes of a protein of this invention. An immunogenic fragment of this invention can comprise, consist essentially of and/or consist of a fusion protein comprising, consisting essentially of and/or consisting of two or more (e.g., 3, 4, 5, 6, 7, 8, 9, 10, etc.) antigenic epitopes. In a preferred embodiment, the immunogenic fragment has a protective effect.

An immunogenic fragment can be any fragment of contiguous amino acids of a protein of this invention and can be for example, 5, 10, 15, 20, 25, 30, 35, 40, 45, 50, 75, 80, 85, 90, 95, 100, 125, 150, 175, 200, 250, 300, 350, 400, 450, 500, 550, 600 or more amino acids in length, including any number between or beyond those numbers recited herein (e.g., 23, 47 or 468 amino acids). Identification of any such immunogenic fragments is routine in the art.

As used herein, the term “polypeptide” or “protein” is used to describe a chain of amino acids that correspond to those encoded by a nucleic acid. A polypeptide of this invention can be a peptide, which usually describes a chain of amino acids of from two to about 30 amino acids. The term polypeptide as used herein also describes a chain of amino acids having more than 30 amino acids and can be a fragment or domain of a protein or a full length protein. Furthermore, as used herein, the term polypeptide can refer to a linear chain of amino acids or it can refer to a chain of amino acids that has been processed and folded into a functional protein. It is understood, however, that 30 is an arbitrary number with regard to distinguishing peptides and polypeptides and the terms can be used interchangeably for a chain of amino acids. The polypeptides of the present invention are obtained by isolation and purification of the polypeptides from cells where they are produced naturally, by enzymatic (e.g., proteolytic) cleavage, and/or recombinantly by expression of nucleic acid encoding the polypeptides or fragments of this invention. The polypeptides and/or fragments of this invention can also be obtained by chemical synthesis or other known protocols for producing polypeptides and fragments.

The amino acid sequences of this invention are presented in the amino to carboxy direction, from left to right. Nucleotide sequences are presented herein by single strand only, in the 5′ to 3′ direction, from left to right. However, it is intended that the nucleic acids of this invention can be either single or double stranded (i.e., including the complementary nucleic acid). A nucleic acid of this invention can be the complement of a nucleic acid described herein.

The term “fragment,” as applied to a polynucleotide, will be understood to mean a nucleotide sequence of reduced length relative to a reference nucleic acid or nucleotide sequence and comprising, consisting essentially of, or consisting of a nucleotide sequence of contiguous nucleotides identical or almost identical (e.g., 75%, 80%, 85%, 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 6, 8, 10, 12, 15, 20, 25, 30, 35, 40, 45, 50, 75, 100, 150, 200, 250, 300, 350, 400, 450, 500 or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 75%, 80%, 85%, 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, 250, 300, 350, 400, 450, 500 or more consecutive amino acids of a polypeptide or amino acid sequence according to the invention.

As used herein, a “functional” polypeptide or “functional protein” or “functional fragment” or “biologically active fragment” of a polypeptide is one that substantially retains at least one biological activity normally associated with that polypeptide (e.g., immunogenic activity, protein binding, ligand or receptor binding, etc.). In particular embodiments, the “functional” polypeptide or protein or “functional fragment” substantially retains all of the activities possessed by the unmodified protein. By “substantially retains” biological activity, it is meant that the polypeptide or protein or fragment retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as protein binding and immunogenic activity can be measured using assays that are well known in the art and as described herein.

A fragment of a polypeptide or protein of this invention can be produced by methods well known and routine in the art. Fragments of this invention can be produced, for example, by enzymatic or other cleavage of naturally occurring peptides or polypeptides or by synthetic protocols that are well known. Such fragments can be tested for one or more of the biological activities of this invention according to the methods described herein, which are routine methods for testing activities of polypeptides, and/or according to any art-known and routine methods for identifying such activities. Such production and testing to identify biologically active fragments and/or immunogenic fragments of the polypeptides described herein would be well within the scope of one of ordinary skill in the art and would be routine.

The term “isolated” can refer to a nucleic acid, nucleotide sequence, polypeptide or fragment thereof that is substantially and/or sufficiently free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or fragment or nucleic acid in a form in which it can be used for the intended purpose (e.g., therapeutically and/or in a diagnostic or detection assay).

An “isolated cell” refers to a cell that is substantially and/or sufficiently separated from other components with which it is normally associated in its natural state. For example, an isolated cell can be a cell in culture medium (e.g., in vitro or ex vivo) and/or a cell in a pharmaceutically acceptable carrier of this invention. Thus, an isolated cell can be delivered to and/or introduced into a subject. In some embodiments, an isolated cell can be a cell that is removed from a subject and manipulated ex vivo and then returned to the subject.

By the terms “express,” “expressing” or “expression” with regard to a nucleic acid comprising a coding sequence, it is meant that the nucleic acid is transcribed, and optionally, translated. Typically, according to the present invention, expression of a coding sequence of the invention will result in production of the polypeptide, fragment, or other product of the invention. The produced polypeptide, fragment or other product can function in intact cells without purification.

As used herein, the term “antibody” includes intact immunoglobin molecules as well as fragments thereof, such as Fab, F(ab′)2, and Fe, which are capable of binding the epitopic determinant of an antigen (i.e., antigenic determinant). Antibodies that bind the polypeptides and fragments of this invention are prepared using intact polypeptides or fragments containing immunogeinic peptides as the immunizing antigen. The polypeptide or fragment used to immunize an animal can be derived from enzymatic cleavage, recombinant expression, isolation from biological materials, synthesis, etc., and can be conjugated to a carrier protein, if desired. Commonly used carriers that are chemically coupled to peptides and proteins for the production of antibody include, but are not limited to, bovine serum albumin, thyroglobulin and keyhole limpet hemocyanin. The coupled peptide or protein is then used to immunize the animal (e.g., a mouse, rat, or rabbit). The polypeptide or peptide antigens can also be administered with an adjuvant, as described herein and as otherwise known in the art.

The term “antibody” or “antibodies” as used herein also refers to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, and/or the antibody can be a chimeric or humanized antibody. See, e.g., Walker et al., Molec. Immunol. 26:403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed e.g., in U.S. Pat. No. 4,676,980. The antibody can further be a single chain antibody or bispecific antibody.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254:1275-1281).

The sample of this invention can be any sample in which a Chlamydia protein or fragment thereof, an antibody to a Chlamydia protein and/or a Chlamydia nucleic acid can be present. For example, the sample can be a body fluid, cells or tissue that can contain a Chlamydia protein, fragment and/or nucleic acid, including but not limited to, vaginal fluid, vaginal tissue, vaginal washing, vaginal swab, urethral swab, urine, blood, serum, plasma, saliva, semen, urethral discharge, vaginal discharge, sputum, bronchoalveolar lavage, joint fluid, cerebrospinal fluid and cells, fluids and/or tissue from any organs to which a Chlamydia protein, fragment and/or nucleic acid can disseminate, including lung, liver, heart, brain, kidney, spleen, muscle, etc., and any combination thereof.

Furthermore the sample can include cells and/or fluid from any site of infection, including but not limited to the cervical canal, uterus, fallopian tubes, testes, anus, throat, lungs, skin, eyes, gastro-intestinal tract, or body cavity. These samples can be used fresh, or stored (frozen, dried, refrigerated, as appropriate) for later use. The samples can be used in their native form (e.g., urine), or processed, extracted, solubilized, or manipulated. Any Chlamydia in these samples can be either viable or dead, whole cell or disrupted, processed or unprocessed.

A “subject” of this invention includes any animal susceptible to infection by a chlamydial species. Such a subject can be a mammal (e.g., a laboratory animal such as a rat, mouse, guinea pig, rabbit, primates, etc.), a farm or commercial animal (e.g., a cow, horse, goat, donkey, sheep, etc.), a domestic animal (e.g., cat, dog, ferret, etc.), an avian species and in particular embodiments, a human. A “subject in need thereof” is a subject known to be, suspected of being, or at risk of being infected with Chlamydia. A subject of this invention can also include a subject not previously known or suspected to be infected by Chlamydia or in need of treatment for Chlamydia infection. For example, a subject of this invention can be administered a composition of this invention even if it is not known or suspected that the subject is infected with Chlamydia or at risk of being infected (e.g., prophylactically). A subject of this invention is also a subject known or believed to be at increased risk of infection by Chlamydia.

By “protect,” “protecting,” and “protection” and like terms it is meant that any level of protection is achieved that is of some benefit to a subject and/or a population of subjects, such that there is a reduction in the incidence and/or the severity and/or the onset of chlamydial infection and/or related disease among treated subjects, regardless of whether the protection is partial or complete.

“Treat,” “treating,” or “treatment” is intended to mean that the inventive methods eliminate, avoid, reduce or delay the incidence and/or onset of the disorder, as compared to that which would occur in the absence of the measure taken. In some embodiments, the present methods slow, delay, control, or decrease the likelihood or probability of chlamydial infection and/or related disease in the subject, as compared to that which would occur in the absence of the measure taken.

“Treat,” “treating,” or “treatment” also refers to any type of action or activity that imparts a modulating effect, which, for example, can be a beneficial effect, to a subject afflicted with a disorder, disease, infection or illness, or at risk of developing a disorder, disease, infection or illness, including improvement in the condition of the subject (e.g., in one or more symptoms), delay in the progression of the condition, delay of the onset of the disorder, disease, infection or illness, and/or change in clinical parameters of the disorder, disease, infection or illness, as would be well known in the art.

“Effective amount” refers to an amount of a composition described herein that is sufficient to produce a desired effect, which can be a therapeutic effect. The exact amount of the composition required for an effective amount will vary from subject to subject, depending on the species, age, weight and general condition of the subject, the severity of the condition being treated, the particular composition used, its mode of administration, the duration of the treatment, the nature of any concurrent treatment, the pharmaceutically acceptable carrier used, and like factors within the knowledge and expertise of those skilled in the art. Thus, it is not possible to specify an exact amount for every composition of this invention. However, an effective amount can be determined by one of ordinary skill in the art in any individual case using only routine experimentation given the teachings herein and by reference to the pertinent texts and literature and/or by using routine experimentation. (See, for example, Remington: The Science and Practice of Pharmacy, 21^{st} Edition (latest edition), Lippincott Williams & Wilkins, Philadelphia, Pa.).

As used herein “effective response” or “responding effectively” means a positive or beneficial response to a particular treatment in contrast to a “lack of an effective response” which can be an ineffectual, negative or detrimental response as well as the lack of a positive or beneficial response. An effective response or lack of effective response (i.e., ineffective response) is detected by evaluation, according to known protocols, of various immune functions (e.g., cell-mediated immunity, humoral immune response, etc.) and pharmacological and biological functions as would be known in the art.

By “prime,” “primed” or “priming” (and grammatical variations thereof) as used herein, it is meant to initiate an active immune response that is less than a fully protective immune response until a second or subsequent dose (booster) is given at a later time.

“Boost” or “booster” means a second or subsequent immunization, after an initial (or “priming”) immunization that enhances the immune response of the subject. Therefore, in some embodiments, the invention provides a composition that produces an anamnestic response against a Chlamydia infection, in a sensitized subject, comprising an anamnestic response-inducing amount of a Chlamydia protein immunizing component. As used herein, the term “anamnestic response” means a secondary (booster) immune response in a sensitized subject. By “sensitized subject” is meant a subject that has previously been in contact with a chlamydial antigen or antigens, either by natural exposure or by vaccination (primary immunization) with Chlamydia protein immunizing components.

As used herein, “detecting” or “detection” means testing, screening or otherwise determining the presence and/or absence of a Chlamydia protein, nucleic acid and/or antibody in a subject. Such detecting or detection can be carried out by methods well known in the art and as described herein.

“Reduce,” “reduced,” “reducing,” and “reduction” (and grammatical variations thereof), as used herein, means a decrease or diminution in a chlamydial infection- or disease-related-parameter that is of some value or benefit (e.g., therapeutic or prophylactic value or benefit).

The terms “vaccine,” “vaccination” or “immunization” are well-understood in the art, and are used interchangeably herein. For example, the terms vaccine, vaccination or immunization can be understood to be a process or composition that increases a subject's immune reaction to immunogen (e.g., by providing an active immune response), and therefore its ability to resist, overcome and/or recover from chlamydial infection and/or related disease.

The terms “protective immunity” or “protective immune response,” as used herein, are intended to mean that the host animal mounts an active immune response to the immunogenic composition and/or that the immunogenic composition provides passive immunity, such that upon subsequent exposure or a challenge, the animal is able to resist or overcome infection and/or disease. Thus, a protective immune response will decrease the incidence of morbidity and/or mortality from subsequent exposure to the pathogen.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” (Herscowitz, Immunophysiology: Cell Function and Cellular Interactions in Antibody Formation, in IMMUNOLOGY: BASIC PROCESSES 117 (Joseph A. Bellanti ed., 1985)). Alternatively stated, an active immune response is mounted by the host after exposure to immunogens by infection and/or by vaccination. Active immunity can be contrasted with passive immunity, which is acquired through the “transfer of preformed substances (antibody, transfer factor, thymic graft, interleukin-2) from an actively immunized host to a non-immune host.” Id.

The term “therapeutically effective amount,” “effective amount” or “immunogenic amount” as used herein, refers to that amount of a composition of this invention that imparts a modulating effect, which, for example, can be a beneficial effect, to a subject afflicted with a condition (e.g., a disorder, disease, infection, syndrome, illness, injury, traumatic and/or surgical wound), including improvement in the condition of the subject (e.g., in one or more symptoms), delay or reduction in the progression of the condition, prevention or delay of the onset of the condition, and/or change in clinical parameters, status or classification of a disease or illness, etc., as would be well known in the art.

### Treatment and Prophylaxis

The methods of this invention can be carried out to treat a subject infected with a chlamydial species and/or to protect a subject against infection and/or disease caused by chlamydial species, including, for example, Chlamydia trachomatis, Chlamydia muridarum and Chlamydia pneumoniae.

Additional aspects of this invention include a method of eliciting an immune response against Chlamydia in a subject, comprising administering to the subject an effective amount of a composition of this invention, thereby eliciting an immune response against Chlamydia in the subject.

Furthermore, the present invention provides a method of inducing immunity against Chlamydia in a subject, comprising administering to the subject an amount of a composition of this invention sufficient to elicit an immune response, wherein said immune response is sufficient to decrease risk of onset of disease caused by Chlamydia.

In addition, the present invention provides a method of eliciting an immune response against Chlamydia in a subject, comprising administering to the subject an effective amount of a composition of this invention comprising, consisting essentially of and/or consisting of a combination (e.g., two or more) of chlamydial proteins and/or immunogenic fragments thereof of this invention, wherein said combination of chlamydial proteins and/or immunogenic fragments has a synergistic effect in eliciting an immune response, thereby eliciting an immune response against Chlamydia in the subject. As used herein, the term “synergistic” is understood to have the art-known meaning of the phenomenon in which the combined action of two or more things (e.g., chlamydial proteins and/or immunogenic fragments thereof) is greater than the sum of their effects individually.

Also provided herein is a method of treating an infection and/or related disease caused by Chlamydia in a subject, comprising administering to the subject an effective amount of a composition of this invention, thereby treating an infection and/or related disease caused by Chlamydia in the subject.

In addition, the present invention provides a method of preventing or reducing the likelihood of infection and/or the development of related disease caused by Chlamydia in a subject, comprising administering to the subject an effective amount of a composition of this invention, thereby preventing or reducing the likelihood of infection and/or the development of related disease caused by Chlamydia in the subject.

The present invention further provides a method of reducing the likelihood of infertility due to Chlamydia infection in a subject, comprising administering to the subject an effective amount of a composition of this invention, thereby reducing the likelihood of infertility due to Chlamydia infection in the subject.

Further provided herein is a method of reducing the incidence of hydrosalpinx due to Chlamydia infection in a subject, comprising administering to the subject an effective amount of a composition of this invention, thereby reducing the incidence of hydrosalpinx due to Chlamydia infection in the subject.

As indicated, in particular embodiments, the methods of the present invention can be practiced to induce an immune response to Chlamydia in a subject. As used herein, the term “induce (or grammatical variations thereof) an immune response against Chlamydia” or “induce immunity against Chlamydia” is intended to encompass the delivery or administration, to a subject of this invention, of agents that induce an immune response against the chlamydial organism itself and/or, e.g., toxins or secreted proteins produced by the chlamydial organism, by means of passive transfer and/or active immune response. In some embodiments, the immune response that is induced can be a protective immune response, for example, in vaccination methods. Protection is not required if there is some other purpose for inducing the immune response, for example, for research purposes or to produce antibody for passive immunization or as a reagent (e.g., to detect, isolate and/or identify Chlamydia species and/or in diagnosis).

The terms “immunogenic amount,” “effective amount,” “effective immunizing amount” or “therapeutically effective amount” as used herein, unless otherwise indicated, mean a dose of a composition of this invention sufficient to induce or elicit an immune response (e.g., protective or therapeutic response) in the immunized subject that is greater than the inherent immunity of non-immunized subjects. An immunogenic amount or effective amount or effective immunizing amount in any particular context can be routinely determined using methods known in the art.

In some embodiments, an effective amount or immunogenic amount or therapeutically effective amount can comprise one or more (e.g., two or three) doses of the composition of this invention so as to achieve the desired immune response.

For example, an effective amount or immunogenic amount can refer to the amount of a composition of this invention that improves a condition (e.g., treats chlamydial infection or related disease) in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

In additional embodiments, the present invention provides a method of providing passive immunity to a subject, comprising administering to the subject an effective amount of an antibody of this invention to the subject.

In addition, the present invention provides a method of treating or preventing infection or disease caused by Chlamydia in a subject comprising contacting an immune cell of the subject with any of the polypeptides, fragments, nucleic acids, vectors and/or antibodies of this invention. The cell can be in vivo or ex vivo and can be, for example, a CD8⁺ T cell which is contacted with the polypeptide and/or fragment of this invention in the presence of a class I MHC molecule, which can be a soluble molecule or it can be present on the surface of a cell which expresses class I MHC molecules. The cell can also be an antigen presenting cell or other class I MI-IC-expressing cell which can be contacted with the nucleic acids and/or vectors of this invention under conditions whereby the nucleic acid or vector is introduced into the cell (e.g., to produce a transformed cell) by standard methods for uptake of nucleic acid and vectors. The nucleic acid encoding the polypeptide and/or fragment of this invention is then expressed and the polypeptide and/or fragment product is processed within the antigen presenting cell or other MHC I-expressing cell and presented on the cell surface as an MHC I/antigen complex. The antigen presenting cell or other class I MHC-expressing cell is then contacted with an immune cell of the subject which binds the class I MHC/antigen complex and elicits an immune response which treats or prevents Chlamydia infection and/or related disease in the subject.

Further embodiments of the present invention include a method of reducing the likelihood of infertility due to Chlamydia infection in a subject, comprising administering to the subject an effective amount of a composition of this invention to the subject. The method can further comprise administering an adjuvant to the subject.

By “reducing the likelihood of infertility due to Chlamydia infection” is meant that a subject of this invention to whom the immunogenic compositions of this invention are administered is prevented from or protected against becoming infertile as a result of Chlamydia infection or that the likelihood that the subject will become infertile as a result of being infected by Chlamydia is reduced as compared to the likelihood that an untreated subject will become infertile as a result of being infected by Chlamydia. The prevention or reduced likelihood or incidence of infertility results from preventing or treating Chlamydia infection in the subject according to the methods of this invention. That infertility is prevented or its likelihood as a result of Chlamydia infection is reduced in a subject can be determined according to protocols described herein and as would be well known in the art.

The present invention further provides a method of reducing the incidence of hydrosalpinx due to Chlamydia infection in a subject, comprising administering to the subject an effective amount of a composition of this invention. The method can further comprise administering an adjuvant to the subject.

Hydrosalpinx is a result of tubal blockade and subsequent retention of fluid exudate within the tubal lumen. Given that the patency of oviducts is important to allow fertilization of the ovum and sperm, and that the hydrosalpinx fluid is toxic to the ovum, the presence of hydrosalpinx serves as an indirect marker of infertility.

By “reducing the incidence of hydrosalpinx due to Chlamydia infection” is meant that a subject of this invention to whom the immunogenic compositions of this invention are administered will be prevented from or protected against developing chlamydial infection and/or developing hydrosalpinx due to Chlamydia infection or has a reduced likelihood of developing hydrosalpinx due to Chlamydia infection or has a lesser degree of hydrosalpinx due to Chlamydia infection as compared to an untreated subject infected by Chlamydia. The prevention or reduced incidence of hydrosalpinx results from preventing or treating (e.g., reducing oviduct pathology) Chlamydia infection in the subject according to the methods of this invention. That hydrosalpinx due to Chlamydia infection is prevented or its incidence and/or degree are reduced in a subject can be determined according to protocols described herein and as would be well known in the art.

In some embodiments, the protein or immunogenic fragment thereof of this invention can be administered to a subject of this invention as a protein or peptide to elicit an immune response to treat and/or prevent chlamydial infections and/or to reduce the likelihood of infertility due to Chlamydia infection and/or to reduce the incidence of hydrosalpinx. In other embodiments, a nucleic acid or multiple nucleic acids encoding a protein or immunogenic fragment thereof of this invention in any combination can be administered to a subject of this invention to elicit an immune response to treat and/or prevent chlamydial infection and/or reduce the likelihood of infertility due to Chlamydia infection and/or to reduce the incidence of hydrosalpinx.

As set forth above, it is contemplated that in the methods wherein the compositions of this invention are administered to a subject or to a cell of a subject, such methods can further comprise the step of administering a suitable adjuvant to the subject or to a cell of the subject. As used herein, a “suitable adjuvant” describes an adjuvant capable of being combined with the polypeptide and/or fragment of this invention to further enhance an immune response without deleterious effect on the subject or the cell of the subject. A suitable adjuvant can be, but is not limited to, CpG, MONTANIDE ISA51 (Seppic, Inc., Fairfield, N.J.), SYNTEX adjuvant formulation 1 (SAF-1), composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion. An adjuvant of this invention can also be an immunostimulatory cytokine, numerous examples of which are described herein and are known in the art.

An adjuvant of this invention, such as, for example, an immunostimulatory cytokine, can be administered before, concurrent with, and/or within a few hours, several hours, and/or 1, 2, 3, 4, 5, 6, 7, 8, 9, and/or 10 days before or after the administration of a composition of this invention to a subject.

Furthermore, any combination of adjuvants, such as immunostimulatory cytokines, can be co-administered to the subject before, after or concurrent with the administration of a composition of this invention. For example, combinations of immunostimulatory cytokines, can include two or more immunostimulatory cytokines of this invention, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules.

The adjuvant can be in the composition of this invention or the adjuvant can be in a separate composition comprising the suitable adjuvant and a pharmaceutically acceptable carrier. The adjuvant can be administered prior to, simultaneous with, or after administration of the composition containing any of the polypeptides, fragments, nucleic acids and/or vectors of this invention. For example, QS-21, similar to alum, complete Freund's adjuvant, SAF, etc., can be administered within days/weeks/hours (before or after) of administration of the composition of this invention.

The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

The compositions of the present invention can also include other medicinal agents, pharmaceutical agents, carriers, diluents, immunostimulatory cytokines, etc. Actual methods of preparing such dosage forms are known, or will be apparent, to those skilled in this art.

The compositions of this invention can be administered to a cell of a subject or to a subject either in vivo or ex vivo. For administration to a cell of the subject in vivo, as well as for administration to the subject, the compositions of this invention can be administered orally, parenterally (e.g., intravenously), by intramuscular injection, by intraperitoneal injection, subcutaneous injection, transdermally, extracorporeally, topically or the like. Also, the compositions of this invention can be pulsed onto dendritic cells, which are isolated or grown from a subject's cells, according to methods well known in the art, or onto bulk peripheral blood mononuclear cells (PBMC) or various cell subtractions thereof from a subject.

The composition of this invention can be administered to a subject prior to, during and/or after Chlamydia infection. As an example, to a subject diagnosed with Chlamydia infection or known or suspected or believed to be at risk of being infected with Chlamydia or in whom it is desirable to induce an immune response to Chlamydia, between about 50-1000 nM, or between about 100-500 nM of a polypeptide and/or immunogenic fragment of this invention can be administered, e.g., subcutaneously, and can be in an adjuvant, at one to three hour/day/week intervals until an evaluation of the subject's clinical parameters indicate that the subject is not infected by Chlamydia and/or the subject demonstrates the desired immunological response. Alternatively, a polypeptide and/or fragment of this invention can be pulsed onto dendritic cells at a concentration of between about 10-100 μM and the dendritic cells can be administered to the subject intravenously at the same time intervals. The treatment can be continued or resumed if the subject's clinical parameters indicate that Chlamydia infection is present and can be maintained until the infection is no longer detected by these parameters and/or until the desired immunological response is achieved.

Parenteral administration of the peptides, polypeptides, nucleic acids and/or vectors of the present invention, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained. See, e.g., U.S. Pat. No. 3,610,795, which is incorporated by reference herein in its entirety.

The efficacy of treating or preventing Chlamydia infection by the methods of the present invention can be determined by detecting a clinical improvement as indicated by a change in the subject's symptoms and/or clinical parameters, as would be well known to one of skill in the art.

An immune response elicited or induced or produced by carrying out the methods of this invention employing a protein and/or immunogenic fragment thereof can be a protective immune response, a cellular immune response, a humoral immune response, a Th1 immune response, a Th2 immune response and any combination thereof.

To stimulate the humoral arm of the immune system, i.e., the production of antigen-specific antibodies, an immunogenic fragment can include at least about 5-10 contiguous amino acid residues of the full-length molecule, or at least about 15-25 contiguous amino acid residues of the full-length molecule, or at least about 20-50 or more contiguous amino acid residues of the full-length molecule, that define one or more (e.g., 2, 3, 4, 5, 6, etc.) epitopes, or any integer between five amino acids and the full-length sequence, provided that the fragment in question retains immunogenic activity, as measured by any art-known assay, such as, e.g., the ones described herein and/or those known in the art.

Regions of a given polypeptide that include an epitope can be identified using any number of epitope mapping techniques, well known in the art. (See, e.g., Epitope Protocols in Methods in Molecular Biology, Vol. 66, Glenn E. Morris, Ed., 1996, Humana Press, Totowa, N,J.). For example, linear epitopes can be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci, USA 81:3998-4002; Geysen et al. (1986) Molec. immunol. 23:709-715, all incorporated herein by reference in their entireties.

Similarly, conformational epitopes are readily identified by determining spatial conformation of amino acids such as by, e.g., x-ray crystallography and 2-dimensional nuclear magnetic resonance. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al., Proc. Natl. Acad. Sci. USA (1981) 78:3824-3828) for determining antigenicity profiles and the Kyte-Doolittle technique (Kyte et al., J. Mol. Biol. (1982) 157:105-132) for hydropathy plots.

Generally, T-cell epitopes that are involved in stimulating the cellular arm of a subject's immune system are short peptides of about 8-25 amino acids, and these are not typically predicted by the above-described methods for identifying humoral epitopes. A common way to identify T-cell epitopes is to use overlapping synthetic peptides and analyze pools of these peptides, or the individual ones, that are recognized by T cells from animals that are immune to the antigen of interest, using, for example, an enzyme-linked immunospot assay (ELISPOT). These overlapping peptides can also be used in other assays such as the stimulation of cytokine release or secretion, or evaluated by constructing major histocompatibility (MHC) tetramers containing the peptide. Such immunogenic fragments can also be identified based on their ability to stimulate lymphocyte proliferation in response to stimulation by various fragments from the antigen of interest.

The pharmaceutical compositions of this invention include those suitable for oral, rectal, topical, inhalation (e.g., via an aerosol) buccal (e.g., sub-lingual), vaginal, rectal, intraurethral, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and/or transdermal administration. The compositions herein may also be administered via a skin scarification method, or transdermally via a patch or liquid. The compositions may be delivered subdermally in the form of a biodegradable material that releases the compositions over a period of time. The most suitable route in any given case will depend, as is well known in the art, on such factors as the species, age, gender and overall condition of the subject, the nature and severity of the condition being treated and/or on the nature of the particular composition (i.e., dosage, formulation) that is being administered.

The amount of chlamydial polypeptide or fragment thereof and/or the amount of chlamydial polypeptide-encoding nucleic acid in each dose is selected as an amount which induces the desired immunological response without significant, adverse side effects. Such an amount will vary depending upon which specific immunogen is employed, whether or not the composition includes an adjuvant or nucleic acid encoding an adjuvant, and a variety of host-dependent factors.

In embodiments in which the chlamydial protein or fragment thereof is delivered directly, each dose can comprise, consist essentially of or consist of about 1-1000 ug of protein, including from about 1-200 ug, as well as from about 10-100 ug. An effective dose of a nucleic acid-based composition will generally involve administration of from about 1-1000 ug of nucleic acid. An optimal amount for a particular composition vaccine can be ascertained by standard studies involving observation of antibody titers and other responses in subjects. The level of immunity elicited or produced can be monitored to determine the need, if any, for boosters. Following an assessment of antibody titers in a sample from the immunized subject, optional booster immunizations may be desired.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year or as necessary to control the condition. In some embodiments, one, two, three or four doses over the lifetime of a subject can be adequate to achieve the desired therapeutic effect. In some embodiments, alternate day dosing can be employed (e.g., every other day). The amount and frequency of administration of the composition of this invention will vary depending on the particular condition being treated or to be prevented and the desired therapeutic effect.

Human disease associated with chlamydial infection that can be treated and/or prevented using the methods and compositions described herein include, but are not necessarily limited to, sexually transmitted disease (urethritis and epidiymitis in men; pelvic inflammatory disease in women), conjunctivitis, and pneumonia. Of particular interest is the treatment, inhibition and/or prevention of infection by C. trachomatis, by C. pneumonia, and by C. psittaci.

C. trachomatis, the most common cause of sexually transmitted diseases in the United States, causes a variety of diseases including nongonococcal urethritis and epididymitis in men; cervicitis, urethritis, and pelvic inflammatory disease in women; Reiter's syndrome; and neonatal conjunctivitis and pneumonia, the latter of which are generally acquired through maternal transmission. C. trachomatis has been implicated in 20% of adults with pharyngitis. Several immunotypes of C. trachomatis can cause lymphogranuloma venereum (LGV), a disease found mostly in tropical and subtropical areas. LGV strains invade and reproduce in regional lymph nodes.

C. pneumoniae (previously called Taiwan acute respiratory agent or TWAR), originally considered a serotype of C. psittaci, can cause pneumonia, especially in children and young adults. The organism has been found in atheromatous lesions, and infection is associated with increased risk of coronary artery disease.

C. psittaci infects many animals, but human infection is closely related to contact with birds. In humans, C. psittaci causes psittacosis, an infectious atypical pneumonia transmitted to humans by certain birds. In humans, psittacosis (omithosis, parrot fever) is usually caused by inhaling dust from feathers or excreta of infected birds or by being bitten by an infected bird; rarely, it occurs by inhaling cough droplets of infected patients or venereally. Human-to-human transmission may be associated with highly virulent avian strains.

The pharmaceutical compositions of this invention include those suitable for oral, rectal, topical, inhalation (e.g., via an aerosol) buccal (e.g., sub-lingual), vaginal (e.g., vaginal ring), rectal, intraurethral, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration. The compositions herein can also be administered via a skin scarification method or transdermally via a patch, liquid or gel. The compositions can be delivered subdermally in the form of a biodegradable material that releases the compositions over time. The most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

### Detection and Diagnosis

The present invention additionally provides a method of detecting an antibody to Chlamydia in a sample, comprising: a) contacting the sample with a composition of this invention under conditions whereby an antigen/antibody complex can form; and b) detecting antigen/antibody complex formation, thereby detecting an antibody to Chlamydia in the sample.

Also provided herein is a method of diagnosing a Chlamydia infection in a subject, comprising: a) contacting a sample from the subject with a composition of this invention under conditions whereby an antigen/antibody complex can form; and b) detecting antigen/antibody complex formation, thereby diagnosing a Chlamydia infection in the subject.

The present invention further provides a method of detecting a Chlamydia protein in a sample, comprising: a) contacting the sample with an antibody that specifically binds a Chlamydia protein or immunogenic fragment thereof of this invention under conditions whereby an antigen/antibody complex can form; and b) detecting antigen/antibody complex formation, thereby detecting a Chlamydia protein in the sample.

In addition, a method is provided of diagnosing a Chlamydia infection in a subject, comprising: a) contacting a sample from the subject with an antibody that specifically binds a Chlamydia protein or immunogenic fragment thereof of this invention under conditions whereby an antigen/antibody complex can form; and b) detecting antigen/antibody complex formation, thereby diagnosing a Chlamydia infection in the subject.

A variety of protocols for detecting the presence of and/or measuring the amount of polypeptides, fragments and/or peptides in a sample, using either polyclonal or monoclonal antibodies specific for the polypeptide, fragment and/or peptide are known in the art.

Examples of such protocols include, but are not limited to, enzyme immunoassays (ETA), agglutination assays, immunoblots (Western blot; dot/slot blot, etc.), radioimmunoassays (RIA), immunodiffusion assays, chemiluminescence assays, antibody library screens, expression arrays, enzyme-linked immunosorbent assays (ELISA), radioimmunoassays (RIA), immunoprecipitation, Western blotting, competitive binding assays, immunofluorescence, immunohistochemical staining precipitation/flocculation assays and fluorescence-activated cell sorting (FACS). These and other assays are described, among other places, in Hampton et al. (Serological Methods, a Laboratory Manual, APS Press, St Paul, Minn. (1990)) and Maddox et al. (J. Exp. Med. 158:1211-1216 (1993)).

The nucleic acids, vectors and/or cells of this invention can also be used in the detection and/or diagnostic methods of this invention according to conventional protocols. For example, a nucleic acid of this invention (e.g., a nucleic acid encoding a protein, immunogenic fragment or homologue of this invention) can be detected by contacting a sample suspected of containing a nucleic acid of this invention with a nucleotide sequence that is fully complementary or sufficiently complementary to the nucleic acid (e.g., as a probe or primer) under conditions whereby a hybridization complex can form and/or an amplification reaction can occur and detecting the formation of the hybridization complex and/or amplification product, thereby detecting the nucleic acid of this invention in the sample. Nucleic acid hybridization protocols and amplification protocols for detection of nucleic acids as well as for diagnosis of infection and disease are well known in the art.

For example, nucleic acid can be obtained from any suitable sample from the subject that will contain nucleic acid and the nucleic acid can then be prepared and analyzed according to well-established protocols. In some embodiments, analysis of the nucleic acid can be carried by sequencing the nucleic acid strand and/or its complement, by hybridization reaction and/or by amplification of a region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). In some embodiments, the amplification product can be visualized directly in a gel by staining or the product can be detected by hybridization with a detectable probe or by translation into a gene product. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides for use as primers and/or probes for detecting and/or identifying nucleic acids of this invention.

### Antibodies

The present invention also provides an antibody that specifically binds a chlamydial polypeptide and/or immunogenic fragment and/or homologue of this invention, as well as a method of making an antibody specific for a polypeptide and/or fragment of this invention comprising: a) immunizing an animal with a polypeptide and/or fragment of this invention under conditions whereby the animal produces antibodies that specifically bind the polypeptide and/or fragment of this invention; and b) removing biological materials comprising the antibodies from the animal. Also provided herein is an antibody produced by the methods set forth herein.

Antibodies of this invention can be generated using methods that are well known in the art. Such antibodies and immunoglobulin molecules of this invention can include, but are not limited to, polyclonal antibodies, monoclonal antibodies, chimeric antibodies, humanized antibodies, single chain antibodies (e.g., scFv), Fab fragments, and fragments produced by a Fab expression library.

In general, techniques for preparing polyclonal and monoclonal antibodies as well as hybridomas capable of producing a desired antibody are well known in the art. Any animal known to produce antibodies can be immunized with a polypeptide, fragment and/or antigenic epitope of this invention. Methods for immunization of animals to produce antibodies are well known in the art. For example, such methods can include subcutaneous or intraperitoneal injection of the polypeptide, fragment and/or antigenic epitope of this invention.

The polypeptide, fragment or antigenic epitope that is used as an immunogen can be modified and/or administered in an adjuvant in order to increase antigenicity. Methods of increasing the antigenicity of a protein or peptide are well known in the art and include, but are not limited to, coupling the antigen with a heterologous protein (such as globulin or β-galactosidase) and/or through the inclusion of an adjuvant during immunization.

For example, for the production of antibodies, various hosts including goats, rabbits, rats, mice, humans, and others, can be immunized by injection with the polypeptides and/or fragments of this invention, with or without a carrier protein. Additionally, various adjuvants may be used to increase the immunological response. Such adjuvants include, but are not limited to, Freund's complete and incomplete adjuvants, mineral gels such as aluminum hydroxide, and surface-active substances such as lysolecithin, pluronic polyols, polyanions, peptides, oil emulsions, keyhole limpet hemocyanin, and dinitrophenol. Nonlimiting examples of adjuvants used in humans include BCG (bacilli Calmette-Guerin) and Corynebacterium parvum.

Polypeptides, peptides and/or fragments of this invention used as antigens to produce the antibodies of this invention can have an amino acid sequence consisting of at least about five amino acids and in certain embodiments, at least about ten amino acids. In one embodiment, the antigen is identical to a portion of the amino acid sequence of the natural protein, and it can contain the entire amino acid sequence of a small, naturally-occurring molecule. Short stretches of the polypeptides and/or fragments of this invention can be fused with all or a fragment of another protein that acts as a carrier protein (e.g., keyhole limpet hemocyanin) and antibodies can be produced against the chimeric polypeptide or peptide.

Monoclonal antibodies to the polypeptides and/or fragments of this invention are prepared using any technique, which provides for the production of antibody molecules by continuous cell lines in culture. These include, but are not limited to, the hybridoma technique, the human B-cell hybridoma technique, and the EBV-hybridoma technique (Kohler et al. 1975. Nature 256:495-497; Kozbor et al. 1985. J. Immunol. Methods 81:31-42; Cote et al. 1983. Proc. Natl. Acad. Sci. 80:2026-2030; Cole et al. 1984. Mol. Cell. Biol. 62:109-120).

For example, to produce monoclonal antibodies, spleen cells from the immunized animal are removed, fused with myeloma cells, and cultured in selective medium to become monoclonal antibody-producing hybridoma cells, according to techniques routine in the art. Any one of a number of methods well known in the art can be used to identify the hybridoma cell, which produces an antibody with the desired characteristics. These include screening the hybridomas by ELISA assay, Western blot analysis, or radioimmunoassay. Hybridomas secreting the desired antibodies are cloned and the class and subclass are identified using standard procedures known in the art.

For polyclonal antibodies, antibody-containing serum is isolated from the immunized animal and is screened for the presence of antibodies with the desired specificity using any of the well known procedures as described herein.

Monoclonal Fab fragments can be produced in bacterial cell such as E. coli by recombinant techniques known to those skilled in the art. See, e.g., W. Huse, (1989) Science 246:1275-81. Antibodies can also be obtained by phage display techniques known in the art or by immunizing a heterologous host with a cell containing an immunogenic peptide or epitope of interest.

The present invention further provides antibodies of this invention in detectably labeled form. Antibodies can be detectably labeled through the use of radioisotopes, affinity labels (such as biotin, avidin, etc.), enzymatic labels (such as horseradish peroxidase, alkaline phosphatase, etc.), fluorescence labels (such as FITC or rhodamine, etc.), paramagnetic atoms, gold beads, etc. Such labeling procedures are well-known in the art. The labeled antibodies of the present invention can be used for in vitro, in vivo, and in situ assays to identify a polypeptide and/or fragment of this invention in a sample.

In some embodiments, the present invention further provides the antibodies and/or antigens of this invention immobilized on a solid support (e.g., beads, plates, slides or wells formed from materials such as, e.g., latex or polystyrene). Nonlimiting examples of such solid supports include polycarbonate, agarose, nitrocellulose, sepharose, acrylic resins, polyacrylamide and latex beads, as well as any other solid support known in the art. Techniques for coupling antibodies and antigens to such solid supports are well known in the art (Weir et al., Handbook of Experimental Immunology 4th Ed., Blackwell Scientific Publications, Oxford, England, Chapter 10 (1986)). Antibodies and/or antigens of this invention can likewise be conjugated to detectable groups such as radiolabels (e.g., ³⁵S, ¹²⁵I, ¹³¹I), enzyme labels (e.g., horseradish peroxidase, alkaline phosphatase), and fluorescence labels (e.g., fluorescein) in accordance with known techniques. Conditions suitable for the formation of an antigen/antibody complex are routine in the art and form the basis for all immunoassays. Such conditions may vary depending on the particular reagents, samples and/or steps employed in a given immunoassay, as would be readily determined by one of ordinary skill in the art. Determination of the formation of an antibody/antigen complex in the methods of this invention can be by detection of, for example, precipitation, agglutination, flocculation, radioactivity, color development or change, fluorescence, luminescence, etc., as is well know in the art.

In addition, techniques developed for the production of chimeric antibodies or humanized antibodies by splicing mouse antibody genes to human antibody genes to obtain a molecule with appropriate antigen specificity and biological activity can be used (Morrison et al. 1984. Proc. Natl. Acad. Sci. 81:6851-6855; Neuberger et al. 1984. Nature 312:604-608; Takeda et al. 1985. Nature 314:452-454). Alternatively, techniques described for the production of single chain antibodies can be adapted, using methods known in the art, to produce single chain antibodies specific for the polypeptides and fragments of this invention. Antibodies with related specificity, but of distinct idiotypic composition, can be generated by chain shuffling from random combinatorial immunoglobin libraries (Burton 1991. Proc. Natl. Acad. Sci. 88:11120-3).

Antibody fragments that specifically bind the polypeptides and/or fragments of this invention can also be generated. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed to allow rapid and easy identification of monoclonal Fab fragments with the desired specificity (Huse et al. 1989. Science 254:1275-1281).

Various immunoassays can be used for screening to identify antibodies having the desired specificity for the proteins and peptides of this invention. Numerous protocols for competitive binding or immunoradiometric assays using either polyclonal or monoclonal antibodies with established specificity are well known in the art. Such immunoassays typically involve the measurement of complex formation between an antigen and its specific antibody (e.g., antigen/antibody complex formation). For example, a two-site, monoclonal-based immunoassay utilizing monoclonal antibodies reactive to two non-interfering epitopes on the proteins or peptides of this invention can be used, as well as a competitive binding assay.

### Kits

It is further contemplated that the present invention provides kits for detection of the polypeptides and/or fragments and/or antibodies of this invention in a sample. In one embodiment, the kit can comprise one or more antibodies of this invention, along with suitable buffers, wash solutions and/or other reagents for the detection of antibody/antigen complex formation. In an alternative embodiment, a kit of this invention can comprise a polypeptide, an antigenic peptide of the polypeptide of this invention, a fragment of this invention and/or an antigenic peptide of a fragment of this invention, along with suitable buffers, wash solutions and/or other reagents for the detection of antibody/antigen complex formation.

The present invention further provides a kit for the detection of nucleic acid encoding the polypeptides and/or fragments of this invention. For example, in one embodiment, the kit can comprise one or more nucleic acids of this invention, along with suitable buffers, wash solutions and/or other reagents for the detection of hybridization complex formation.

It would be well understood by one of ordinary skill in the art that the kits of this invention can comprise one or more containers and/or receptacles to hold the reagents (e.g., antibodies, antigens, nucleic acids) of the kit, along with appropriate buffers and/or wash solutions and directions for using the kit, as would be well known in the art. Such kits can further comprise adjuvants and/or other immunostimulatory or immunomodulating agents, as are well known in the art.

The following examples are included to demonstrate various embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### EXAMPLES

### Example I

### Genome Wide Profiling of the Humoral Immune Response to C. trachomatis Infection Reveals Vaccine Candidate Antigens Expressed in Humans

A whole genome scale proteome array consisting of 934 fusion proteins representing 909 unique ORFs encoded in the C. trachomatis genome and plasmid was used to profile anti-chlamydial antibody responses. A total of 719 chlamydial proteins were recognized by one or more antisera from 99 women urogenitally infected with C. trachomatis. Twenty-seven of the 719 antigens were recognized by 50% human antisera with significant binding intensity, thus designated as immunodominant antigens. Comparison of antigen profiles recognized by live chlamydial organism-infected versus dead organism-immunized hosts has led to the identification of infection-dependent or in vivo expressed antigens. The infection-dependent antigens induced antibodies only during live infection but not after immunization with inactivated organisms. Many of these antigens, including some immunodominant ones, were highly expressed during chlamydial intracellular replication but only minimally packaged into the infectious elementary bodies. Since inactivated whole chlamydial organism-based vaccines failed to induce protective immunity in humans, identification of infection-dependent or in vivo expressed immunodominant antigens in humans greatly facilitates the selection of chlamydial subunit vaccine candidates. The ability of the whole genome scale proteome array approach to detect both linear and conformation-dependent antibodies has revealed a large C. trachomatis ANTIGENome in humans and identified novel vaccine candidate antigens from C. trachomatis. This approach is also applicable to other pathogens.

### Chlamydial Organisms and Chlamydial Infection in Cell Culture

C. trachomatis serovar D organisms were grown, purified and titrated as previously described (32). The EB organisms were purified from 48 h culture while the RB organisms were from 24 h culture. Aliquots of the purified organisms were stored at −80° C. until use. HeLa cells (ATCC, Manassas, Va. 20108) were maintained in DMEM (GIBCO BRL, Rockville, Md.) with 10% fetal calf serum (FCS; GIBCO BRL) at 37° C. in an incubator supplied with 5% CO₂. To prepare chlamydial infection samples for various assays, HeLa cells grown in tissue culture flasks or on glass coverslips in 24 well plates were pretreated with DMEM containing 30 ug/ml of DEAE-Dextran (Sigma, St Luis, Mo.) for 10 min. After removal of the DEAE-Dextran solution, chlamydial organisms diluted in DMEM were added to the cell monolayers and the organisms were allowed to attach for 2 hours at 37° C. The infected cells were continuously cultured in DMEM with 10% FCS and 2 ug/ml of cycloheximide (Sigma) and processed at various time points after infection as indicated in individual experiments. The infectious dose was pre-titrated and an infection rate of ˜50% or less was applied to samples for immunofluorescence assays or 90% or higher for Western blot assays or making whole cell lysates.

### Cloning Chlamydial Genes and Expressing Chlamydial Proteins.

All open reading frames (ORFs) encoded by C. trachomatis serovar D genome and the cryptic plasmid were cloned into a pGEX vector system (Amersham Biosciences Corp, Piscataway, N.J.). This vector system allows the protein of interest to be expressed as a fusion protein with the glutathione-S-transferase (GST) fused to the N-terminus of the chlamydial proteins (19,34). Protein expression was induced with isopropyl-beta-D-thiogalactoside (IPTG; Invitrogen, Carlsbad, Calif.). To ensure that each fusion protein is produced with adequate quantity and quality, protein induction conditions were individually optimized using the following variables: IPTG concentration (from 0 to 5 mM), starting bacterium number (OD=0.3 to 1.5), incubation temperature (10° C. to 37° C.) and time (0.5 h to overnight). After protein induction, bacteria were harvested and the pellets resuspended in a Triton lysis buffer [1% Triton X 100, 1 mM PMSF, 75 IU/ml of Aprotinin, 20 uM Leupeptin and 1.6 uM Pepstatin in PBS (phosphate-buffered saline at pH 7.5)], followed by short pulses of sonication on ice. The bacterial lysates, after a high-speed centrifugation to remove debris, were aliquoted and stored at −80° C. The quality of the expressed fusion proteins was assessed by purifying the fusion proteins from a portion of the lysates using glutathione-conjugated agarose beads (Amersham Biosciences Corp). The fusion proteins were checked on SDS-polyacrylamide gels stained with a Coomassie blue dye (Sigma). The bacterial lysate samples that showed a prominent band with the expected molecular weight were used for the subsequent microplate array assays.

All ORFs encoded by C. trachomatis serovar D genome and its cryptic plasmid have been cloned. These are 875 ORFs (designated as CT001 to CT875) plus an additional 20 ORFs identified after the genome annotation, including CT039.1, CT172.1, CT221.1, CT326.1, CT326.2, CT357R, CT382.1, CT421.1, CT421.2, CT444.1, CT480.1, CT496.1, CT593.1, CT606.1, CT638.1, CT652.1, CT794.1, CT814.1 & CT849.1 (35). In addition, during genome sequence analysis and gene cloning, six additional potential ORFs from five known gene sequences were identified and they are designated as CT150b, CT235b, CT743b, CT810b, CT810c and CT814b. Furthermore, (36) predicted 10 more ORFs from the previously determined intergenic regions of serovar D genome and they are designated as DEG02, DEG03, DEG04, DEG07, DEG12, DEG13, DEG14, DEG15A, DEG15B & DEG16. Finally, the plasmid encodes 8 ORFs. Thus, a total of 919 genes were cloned. However, due to protein insolubility, some ORFs had to be recloned and expressed in smaller fragments. As a result, 789 ORFs were expressed in full length and 120 in one or more fragments. The remaining 10 ORFs were not expressed at all despite best efforts. These difficult ORFs include CT081 (98 amino acids, unknown function), CT219 (302aa with 8 transmembrane domains, 4-hydroxybenzoate octaprenyltransferase or UbiA), CT267 (100aa, histone-like DNA binding protein or IhfA), CT786 (45aa, 50S ribosomal protein L36 or RL36), CT039.1 (50aa), CT221.1 (46aa), CT480.1 (54aa), CT814.1 (120aa), DEG02 (154aa) and DEG16 (113aa). It is possible that some of these ORFs may not code for any proteins by chlamydial organisms. Nevertheless, protein expression conditions have been optimized for 909 of the 919 ORFs in 934 bacterial expression clones (some ORFs were expressed in multiple fragments, thus multiple clones). These 934 bacterial lysates representing 909 unique Chlamydia trachomatis proteins were produced for setting up the whole genome scale protein array.

Arraying Chlamydial Proteins onto Microplates Pre-Coated with Glutathione.

Bacterial lysates containing the fusion proteins were added to glutathione-coated 96 well microplates (Pierce, Rockford, Ill.) at a 1:10 dilution in PBS with a total volume of 200 ul/well. The plates were incubated overnight at 4° C. to allow GST fusion proteins to bind to the plate-immobilized glutathione. To minimize the variations in quantity of fusion proteins captured onto the plates between the lysate samples, an excessive amount of each fusion protein was used to saturate the glutathione-coated assay plates. It was determined that 20 ul bacterial lysate per well was sufficient for saturating the assay plate if the amount of full-length fusion protein precipitated from the 20 ul bacterial lysate was visible on a SDS gel after Coomassie blue staining. After blocking with 2.5% milk in PBS and washing with PBST (PBS with 0.05% Tween; Sigma), the plates were ready for use.

### Chlamydial Fusion Protein-Arrayed Microplate Enzyme-Linked Immunosorbent Assay (ELISA).

The ELISA was carried out as descried previously (33). Briefly, all serum and antibody samples were pre-absorbed with bacterial lysates in order to prevent the detection of cross-reactive antibodies (human or animal sera may contain antibodies reactive with bacterial antigens that may contaminate the microplate wells). The bacterial lysates used for preabsorption were prepared in the same way as the fusion protein-containing lysates except that XL1-blue bacteria were transformed with the pGEX-6p-2 vector alone. After pre-absorption, all serum samples were titrated for recognizing chlamydial antigens using an immunofluorescence assay (17,33). The serum samples used in the current study meet the following criteria: The positive serum samples from infected or immunized hosts detected chlamydial antigens in Chlamydia-infected cultures with a minimum titer of 1:1000 while the control human sera and pre-bleeding animal sera failed to detect chlamydial antigens even at 1:50 dilution. For proteome array ELISA, the pre-absorbed serum samples were diluted in PBS containing 10% FCS. The primary antibody binding was permitted for 2 hrs at RT and visualized with horse radish peroxidase (HRP)-conjugated goat anti-human, mouse or rabbit IgG (Jackson ImmunoResearch Laboratories, Inc., West Grove, Pa.) in combination with substrate ABTS (2,2′azino-di-(3-ethylbenzthiozoline sulfonic acid, Sigma). The human antibody binding to chlamydial fusion proteins was quantitated by measuring the absorbance (OD) at 405 nm using a microplate reader (Molecular device, Ramsey, Minn.). The OD values that were 4 fold higher than that in the negative control well (coated with GST-alone lysate) in the same plate were considered positive. All positively detected antigens were re-measured with the same antibody samples with or without further absorption with lysates made from either HeLa cells alone or C. trachomatis serovar D-infected HeLa cells. The antibody binding that was still positive after HeLa-alone lysate absorption but significantly blocked by Chlamydia-infected HeLa lysate absorption was considered positive and the corresponding ODs obtained from the HeLa alone lysate absorption wells were used to represent the binding intensity of the corresponding antibody-antigen pairs.

Collecting Serum Samples from Humans and Producing Antibodies in Mice and Rabbits.

Patient sera were collected from women diagnosed with C. trachomatis cervical infections at the San Antonio Project SAFE research clinic. Women enrolled in a 5-year follow-up study were screened semi-annually for sexually transmitted infections, including chlamydial infection. The diagnosis was based on the detection of C. trachomatis-specific nucleic acids in endocervical secretions using a ligase chain reaction without distinguishing the serotypes of the organisms (Abbott LCX, Abbot Laboratories, Chicago, Ill.). The sera were collected at the time of clinic visits and stored in aliquots at −20° C. The human sera used in the current study were all from the initial visit when patients were detected positive for C. trachomatis DNA in vaginal swab samples and diagnosed with acute cervicitis by a physician. An IRB exempt permit is in place for the current study. Eight sera from healthy female individuals (without C. trachomatis infection) were used as negative controls. For some experiments, chlamydial antigen-specific human antibodies were purified from the human antiserum samples (19). Briefly, the corresponding human antisera were pooled and reacted respectively with following chlamydial fusion proteins, including GST-CT022, GST-CT067, GST-CT101, GST-CT142, GST-CT240, GST-CT695C, GST-CT798C, GST-CT806, GST-CT828 and GST-pCT03 (Pgp3), all of which were pre-immobilized onto glutathione-agarose beads. The bead-bound human antibodies were eluted using a high salt solution (from 200 to 900 mM NaCl). The eluted human antibodies were used to label the corresponding endogenous chlamydial antigens in the immunofluorescence assay as described herein. The high salt elution conditions used in this experiment only eluted human antibodies from glutathione-beads without affecting the binding of GST fusion proteins to the bead-conjugated glutathione. Free glutathione is usually required for competing off the GST fusion proteins.

Animals were either infected with live chlamydial organisms or immunized with inactivated organisms or fusion proteins as previously described (37,38,39,40,41,26). All animals were pre-bled prior to infection or immunization. To generate antibodies for detecting individual chlamydial proteins, chlamydial GST fusion proteins were purified using glutathione-conjugated beads. The bead-bound fusion proteins were cleaved off with a precision protease (Pharmacia) to generate soluble tag-free proteins. The tag-free proteins were concentrated via centricon (Millipore, Billerica, Mass.) and used to immunize mice for antibody production. To produce antibodies to the whole chlamydial organisms, rabbits or mice were immunized with UV-inactivated C. trachomatis servar D organisms emulsified in equal amounts of Incomplete Freund's Adjuvant (IFA). Rabbits were injected intramuscularly with 2×10⁷ IFUs for each injection while mice were injected intraperitoneally with 10⁶ IFUs. Three injections were given to each animal with a three-week interval between each injection and serum samples were collected 2 weeks after the last injection. To infect mice, 1×10⁵ live C. trachomatis organisms in 20 ul SPG were inoculated into mouse nostrals (intranasally, m) or vaginal canal (intravaginally, iv). Successful infection was confirmed by recovering infectious organisms in the lung or vaginal swab samples on day 7 after infection (34). Thirty days after infection, mouse blood was collected.

### Immunofluorescence Staining Assays

The C. trachomatis serovar D-infected HeLa monolayers grown on coverslips were processed for antibody staining (38,32). For detecting conformation-dependent antibodies, the Chlamydia-infected cells were fixed with 2% paraformaldehyde for 30 min, followed by permeabilization with 0.5% Saponin for another 30 min at RT. For detecting denatured proteins, the infected cells were fixed with 50% methanol in PBS for 15 min. After blocking with 5% milk, both the affinity-purified mono-specific human antibodies and a rabbit antiserum raised with the whole organisms were used to costain the cell samples. In some experiments, the affinity-purified human antibody preps were pre-absorbed with chlamydial fusion proteins or GST alone immobilized onto the glutathione agarose beads prior to adding to the cell samples. The primary antibody bindings were visualized with a donkey anti-human IgG conjugated with Cy3 (red) and a donkey anti-rabbit IgG conjugated with Cy2 (green). DNA was labeled with Hoechst dye (blue; Sigma). The images were acquired under an Olympus AX-70 fluorescence microscope (Olympus, Seattle, Wash.) using the software SimplePCI as single color (gray) and merged as tri-color images.

### Immunoprecipitation and Western Blot Assays

Immunoprecipitation and Western blot assays were carried out as described (42,43,44,45,46). Briefly, protein A/G agarose beads were used to immobilize antibodies from human serum samples and the bead-bound human antibody complexes were used to precipitate chlamydial antigens from Chlamydia-infected cell lysates. The cell lysates were prepared by sequential lysis and extraction of HeLa cells with or without Chlamydia infection in MLB buffer (25 mM HEPES pH7.4, 150 mM NaCl, 1% Igepal (sigma), 10 mM MgCl₂, 1 mM EDTA, 10% glycerol, 1 mM PMSF, 75 ug/ml of aprotinin, 20 uM Leupeptin & 1 mM Na3Vo3). For Western blot, the whole cell lysate samples with or without human antibody precipitation along with the purified chlamydial RB and EB organisms were loaded into corresponding SDS polyacrylamide gel wells. After electrophoresis, the proteins were transferred to nitrocellulose membranes and the blots were detected with mouse antibodies raised with the corresponding chlamydial fusion proteins. The mouse antibodies used include monoclonal antibody (mAb) 100a against CPAF (17), anti-CT022, anti-CT067, anti-CT101, anti-CT143, anti-CT208, anti-CT222, anti-CT341, anti-CT403, anti-CT437, anti-CT443, anti-CT456, anti-CT529, anti-CT541, anti-CT610, anti-CT681 (serovar D MOMP), anti-CT694, anti-CT771, anti-CT798, anti-CT806, anti-CT813, anti-CT823, anti-CT828 and anti-pCT03 (Pgp3). The primary antibody binding was probed with an HRP (horse radish peroxidase)-conjugated secondary antibody (Mouse TrueBlot™, ULTRA, cat#18-8817-33, eBioscience, San Diego, Calif. 92121) and visualized with an enhanced chemiluminescence (ECL) kit (Santa Cruz Biotechnology, Inc., Santa Cruz, Calif.).

### Statistical Analysis.

ANOVA Test http://www.physics.csbsju.edu/stats/anova.html) was performed to analyze data from multiple groups and a two-tailed Student t test (Microsoft Excel) was used to compare the means between two groups. A Chi squared test was used for comparing the rate of incidence between two groups.

Mapping B Cell ANTIGENome and Identifying Immunodominant Antigens in Women Urogenitally Infected with C. trachomatis.

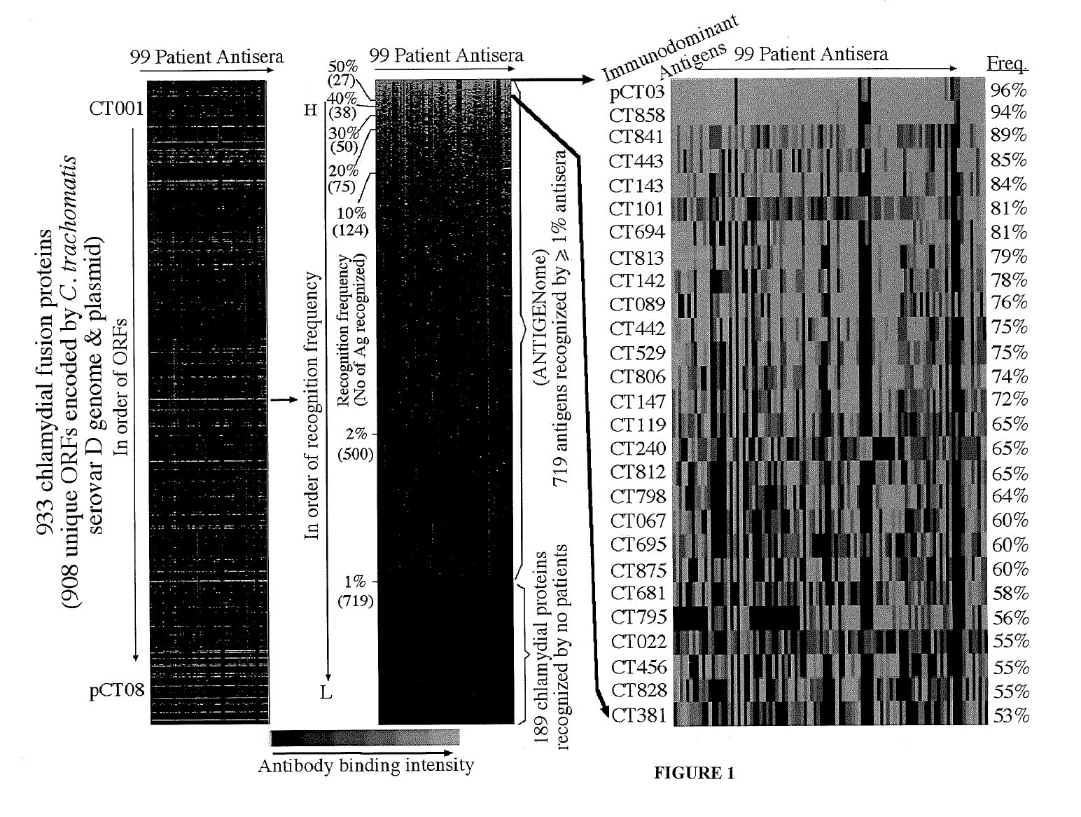

A total of 934 GST fusion proteins representing 909 unique ORFS encoded by Chlamydia trachomatis serovar D genome and plasmid were arrayed onto ten 96 well microplates for assaying human antibody recognition of chlamydial proteins using an ELISA. The antisera from 99 women urogenitally infected with C. trachomatis detected chlamydial proteins that are distributed across the entire chlamydial genome although some chlamydial proteins were more frequently detected than others (FIG. 1, left panel). There was great variation in both the number and type of antigens recognized among the human antisera, with some recognizing many of the 909 antigens and others only being able to recognize a few. When the antigens were rearranged according to recognition frequency (middle panel), 719 out of the 909 ORF-encoded chlamydial proteins were recognized by at least one of the 99 human antisera. The 719 antigens thus form the B cell ANTIGENome of C. trachomatis as defined with these patient antisera. Among the 719 antigens, 124 were recognized by 10%, 75 by 20%, 50 by 30% and 38 by 40% or more antisera respectively. The 27 proteins recognized by 50% or more antisera with an average OD of 0.2 or above were designated as immunodominant antigens (right panel). These 27 immunodominant antigens cover a wide range of proteins including those localized in the organism membrane such as CT681 (MOMP), CT443 (OmcB), CT442, CT812 (PmpD), pCT03 (Pgp3) and the ABC transporters CT067 and CT381, proteins in the inclusion membrane such as CT119 (IncA), CT147, CT442, CT529 and CT813, and proteins secreted into host cell cytosol such as CT858 (CPAF) and pCT03 (Pgp3). In addition, some type III secretion system-related proteins such as CT089 and CT456, metabolic enzymes and proteases such as CT240 (recR), CT798 (glgA), CT806 (ptr), CT828 (nrdB) and CT841 (FtsH) as well as various hypothetical proteins are also among the immunodominant antigens.

### Human Antibody Binding to the Chlamydial Fusion Proteins is Specific and the Fusion Protein-Based Proteome Array Detects Both Linear and Conformation-Dependent Human Antibodies.

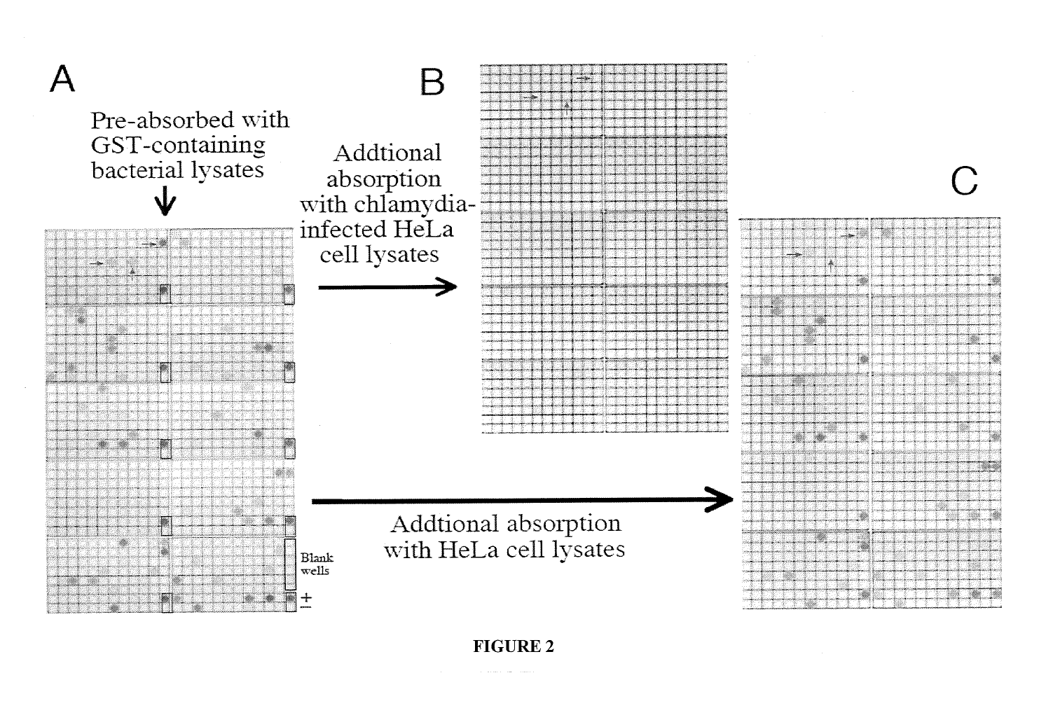

The specificity of the human antibody binding to the arrayed fusion proteins was confirmed at each individual serum level using both absorption and Western blot approaches. In addition, each human antiserum was pre-absorbed with bacterial lysates containing GST alone to remove antibodies that can recognize GST or other bacterial components. Shown in FIG. 2 are the pre-absorption and absorption results for one of the 99 antisera. After pre-absorption, this particular antiserum recognized a total of 54 GST fusion proteins, each representing a unique C. trachomatis ORF (panel A). To confirm the binding specificity, portions of the pre-absorbed antiserum were further absorbed with either Chlamydia-infected (containing endogenous chlamydial proteins, panel B) or normal HeLa lysates (C) prior to reacting with the microplate-arrayed fusion proteins. The absorption with Chlamydia-infected HeLa lysates blocked antibody binding to all fusion proteins while a similar absorption with HeLa alone lysates failed to do so. Individual serum samples from five health women were used to react with the fusion proteins and no significant binding was detected. These observations have demonstrated that the antibody binding to the arrayed fusion proteins detected with the bacterial lysates-preabsorbed human antisera is specific to chlamydial antigens.

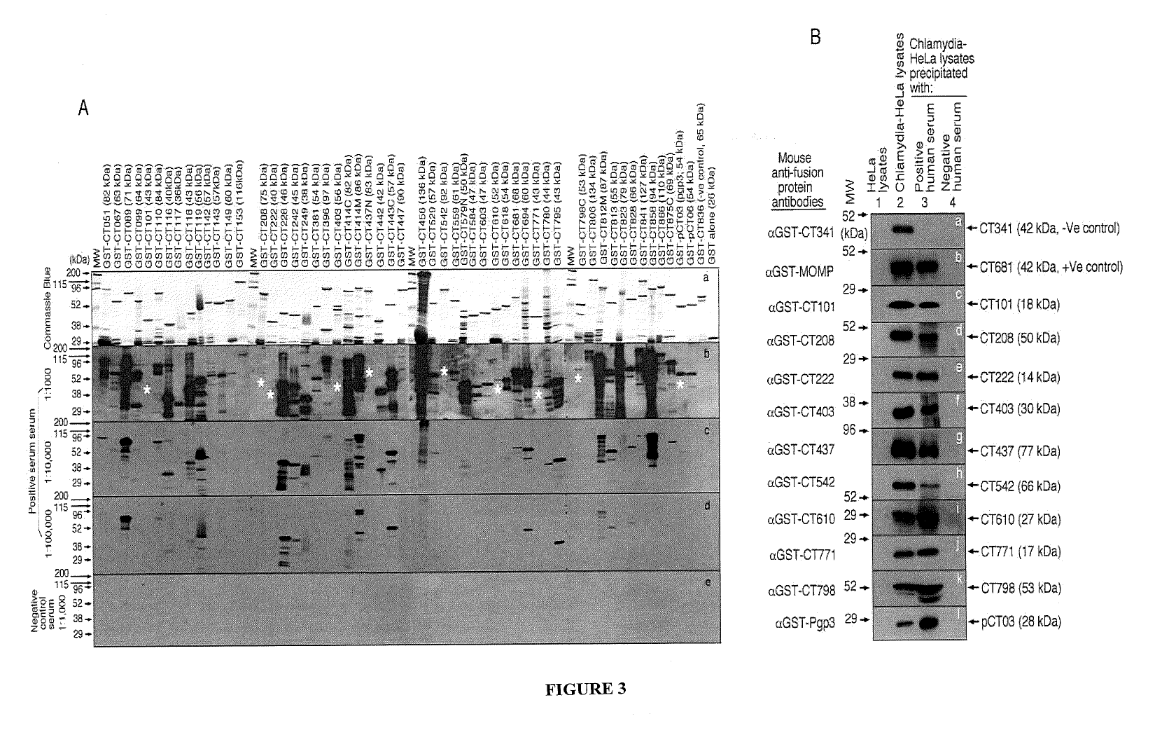

For each antiserum sample, the human antibody binding to the positive antigens was further visualized using a Western blot (FIG. 3). Most of the 54 GST fusion proteins recognized by this particular antiserum sample in the proteome array ELISA assay were also detected by the same antiserum in Western blot assay, indicating that most chlamydial antigen-specific antibodies in the human serum sample are able to react with the SDS-denatured proteins. However, 10 of the 54 antigens were not or only minimally detected by the serum sample in Western blot (marked with a white star in FIG. 3), suggesting that human antibody recognition of the 10 antigens may be dependent on antigen conformation. The conformation may be maintained under proteome ELISA but not Western blot assay conditions. To test this, the same human antiserum sample was used to precipitate endogenous chlamydial antigens from Chlamydia-infected HeLa cell lysates under a non-denaturing condition and then the precipitated antigens were detected in Western blot with the corresponding mouse anti-GST fusion protein antibodies (FIG. 3).

Human antibodies that are affinity-purified with the corresponding chlamydial fusion proteins were used to detect endogenous chlamydial antigens in Chlamydia-infected cells in an immunofluorescence assay. The Chlamydia-infected cells were fixed with either paraformaldehyde (to preserve protein conformation) or methanol (to denature proteins) as described herein. The affinity-purified mono-specific human antibody binding to the endogenous chlamydial antigens was visualized with a goat anti-human IgG conjugated with Cy3 and the DNA was detected with DAPI. All ten human antibodies only recognized antigens in the paraformaldehyde-preserved but not methanol-fixed samples, indicating these human antibodies are conformation-dependent (data not shown).

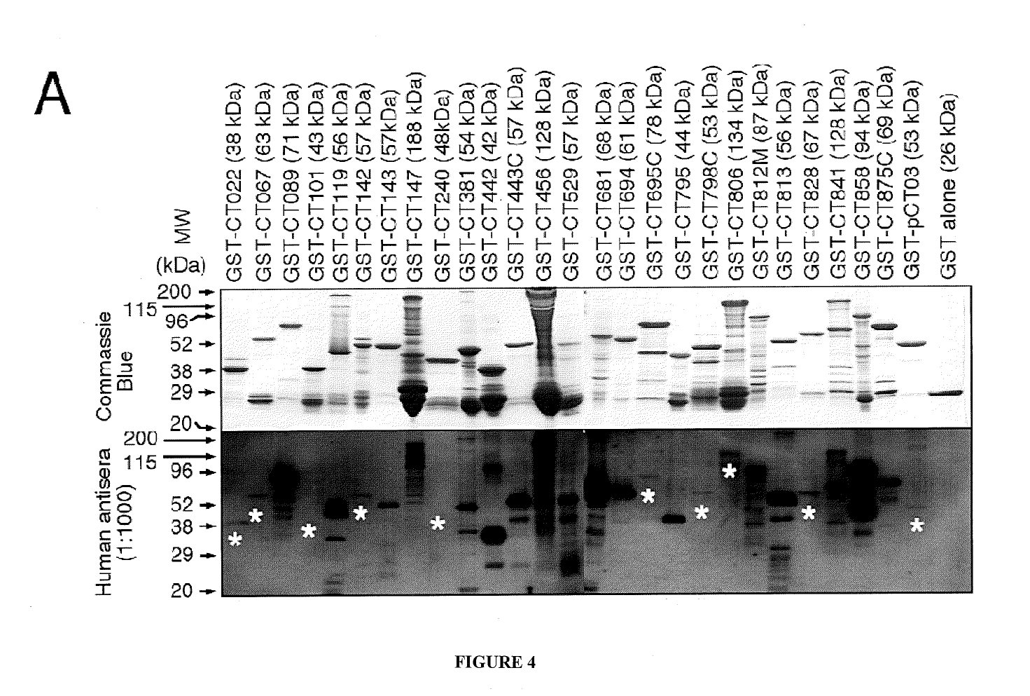

All ten antigens were successfully precipitated by the human antiserum and the amounts of antigens precipitated relative to the amounts of antigens available in the infected cell lysates largely reflected the OD values obtained from the proteome array ELISA. Thus, this human antiserum recognized 10 chlamydial antigens in conformation-dependent manner. These antigens were defined as conformation-dependent. When the 27 immunodominant antigens were similarly analyzed, 10 were no longer detectable by the same antisera after linearization of the antigens in Western blot (FIG. 4A), suggesting that these 10 antigens may be conformation-dependent. To test whether the corresponding human antibodies really require chlamydial antigen conformation for recognition, the agarose bead-immobilized chlamydial fusion proteins were used to affinity-purify the corresponding human antibodies and the purified human mono-specific antibodies were applied to endogenous chlamydial antigens. The affinity-purified human antibodies only recognized the well preserved native-like but not methanol-denatured antigens in Chlamydia-infected cells (FIG. 4B). The above observations all together have demonstrated that the GST fusion protein-based proteome array ELISA can specifically detect both linear and conformation-dependant antibodies.

Comparison of C. trachomatis Antigen Profiles Recognized by Infected Versus Immunized Hosts Reveals Infection-Dependent Antigens.

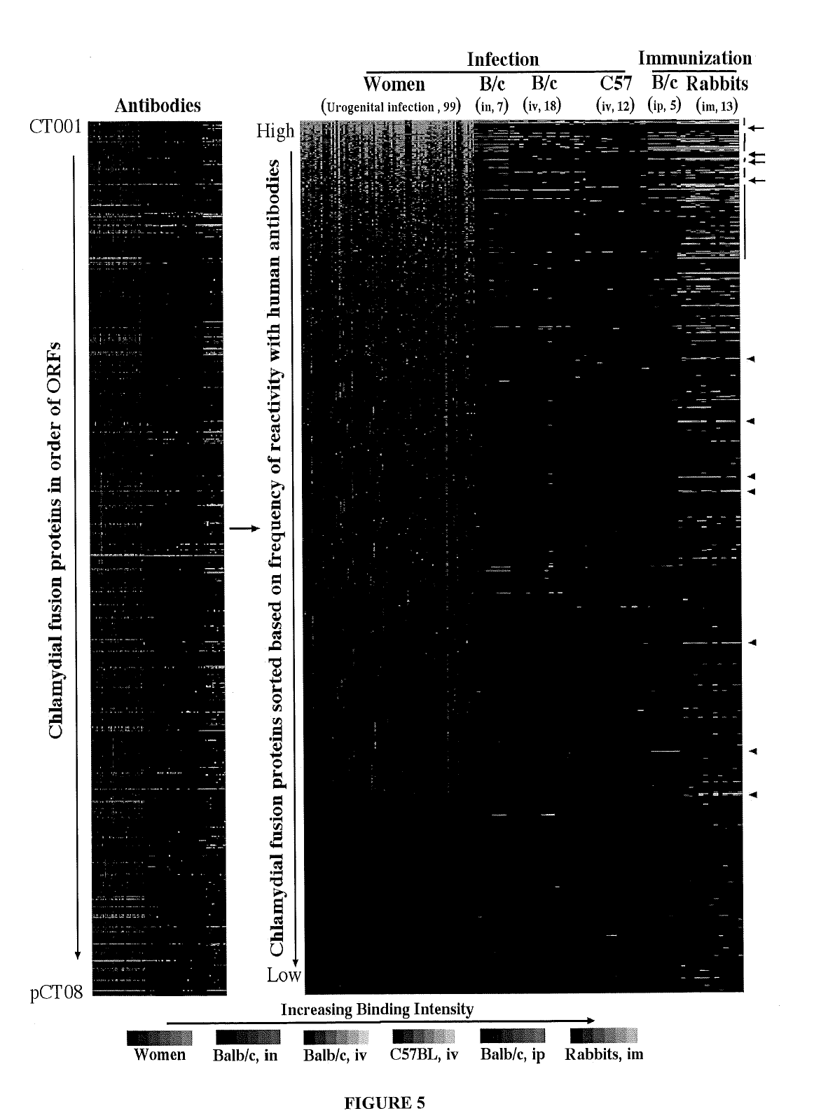

The human-recognized C. trachomatis antigens were compared with those detected by antisera from rabbits and different strains of mice (FIG. 5), since these hosts are frequently used to produce antibodies to C. trachomatis antigens and/or to identify/evaluate chlamydial vaccine candidate antigens. When all possible antigens were considered (as long as an antigen was recognized by at least one antiserum from a given species), it was found that the antisera from 13 immunized rabbits recognized a total of 173 antigens and antisera from 42 mice, regardless of infection and immunization routes, recognized 100. When the antigen profiles were compared at the recognition frequency of 20% or more, the numbers of antigens recognized by the infected humans and immunized rabbits were 75 and 88, respectively, while mice only recognized 18 antigens. This trend continued when analyzed at the frequency of 50% or higher, with 27 by humans, 45 by rabbits and 3 by mice. In a comparison of the types of antigens recognized by the 3 host species, it was found that many human-recognized antigens were also detected by the two rodents species (FIG. 5, marked with vertical bars), suggesting that rodents can be used to study chlamydial immunology and evaluate chlamydial vaccine antigens. Nevertheless, there were also major differences in the types of antigens recognized by the different host species. The most obvious difference was between antigen profiles recognized by the infected humans and immunized rabbits. Some antigens predominantly detected by the immunized rabbits were only minimally recognized or not recognized by human antisera (FIG. 5), while the other antigens recognized by infected humans and mice were not detected with antisera from immunized animals (FIG. 5, marked with arrows), suggesting that expression of these antigens is dependent on live chlamydial infection. This assumption is consistent with a previous observation that the Chlamydia-secreted protein CPAF is not associated with the purified organisms and only expressed during live infection. Thus, anti-CPAF antibodies were only detected in live organism-infected but not dead organism-immunized mice (19). Indeed, CPAF was found to be an immunodominant antigen in both humans and mice infected with live chlamydial organisms but no anti-CPAF antibody was detected in dead organism-immunized rabbits or mice. Thus, by comparing the antigen profiles recognized by live organism-infected versus dead organism-immunized hosts, antigens that are expressed during live infection can be identified. These proteins are designated as infection-dependent antigens.

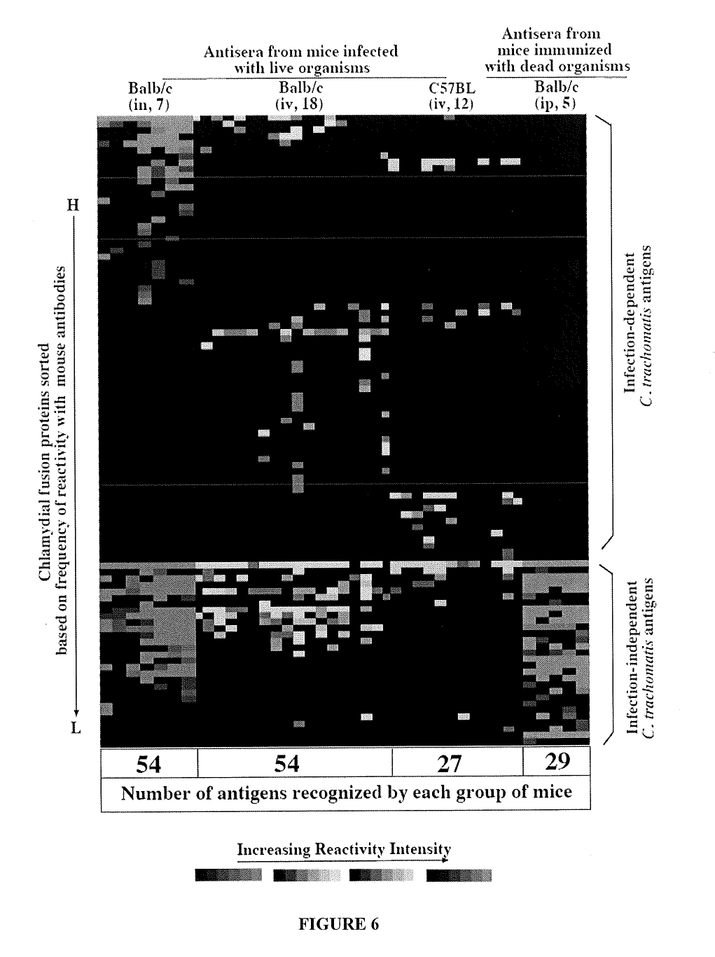

A more careful analysis of antigens recognized by the 42 mice revealed a correlation between the number of antigens recognized by antibodies and the severity of infection (FIG. 6). Intranasal or intravaginal infection of Balb/c mice both induced antibodies to 54 chlamydial antigens, respectively, while intravaginal infection of C57 mice induced antibodies to 27 antigens only. Balb/c mice are known to be more susceptible to chlamydial infection than C57BL mice. More severe or successful infection may allow chlamydial organisms to produce more antigens that can be presented to host immune system. This is supported by the observation that the susceptible Balb/c mice only produced antibodies to 29 chlamydial antigens when dead organisms were used to immunize the mice. This is probably due to the fact that dead organisms are no longer able to express any more proteins even in susceptible hosts. Comparison of the antigens recognized by live organism-infected versus dead organism-immunized mice has led to the identification of infection-dependent antigens, many of which overlap with those identified with human antibodies. The antigens recognized by both the immunized and infected mice are likely associated with the purified EB organisms and antibody production to these antigens is not necessarily dependant on live infection, thus defining them as infection-independent antigens.

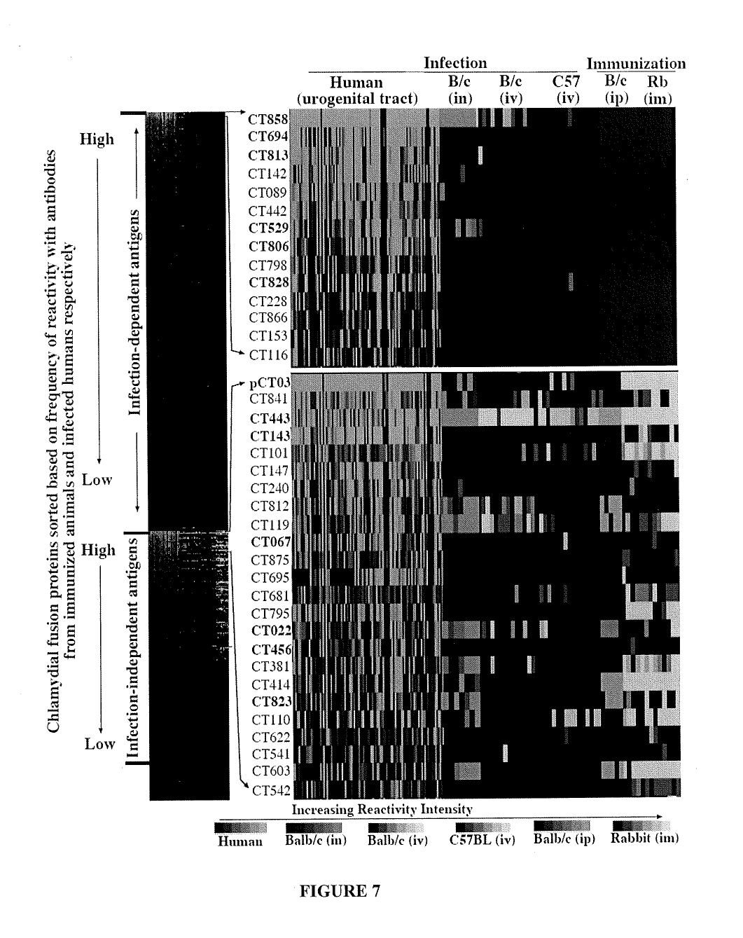

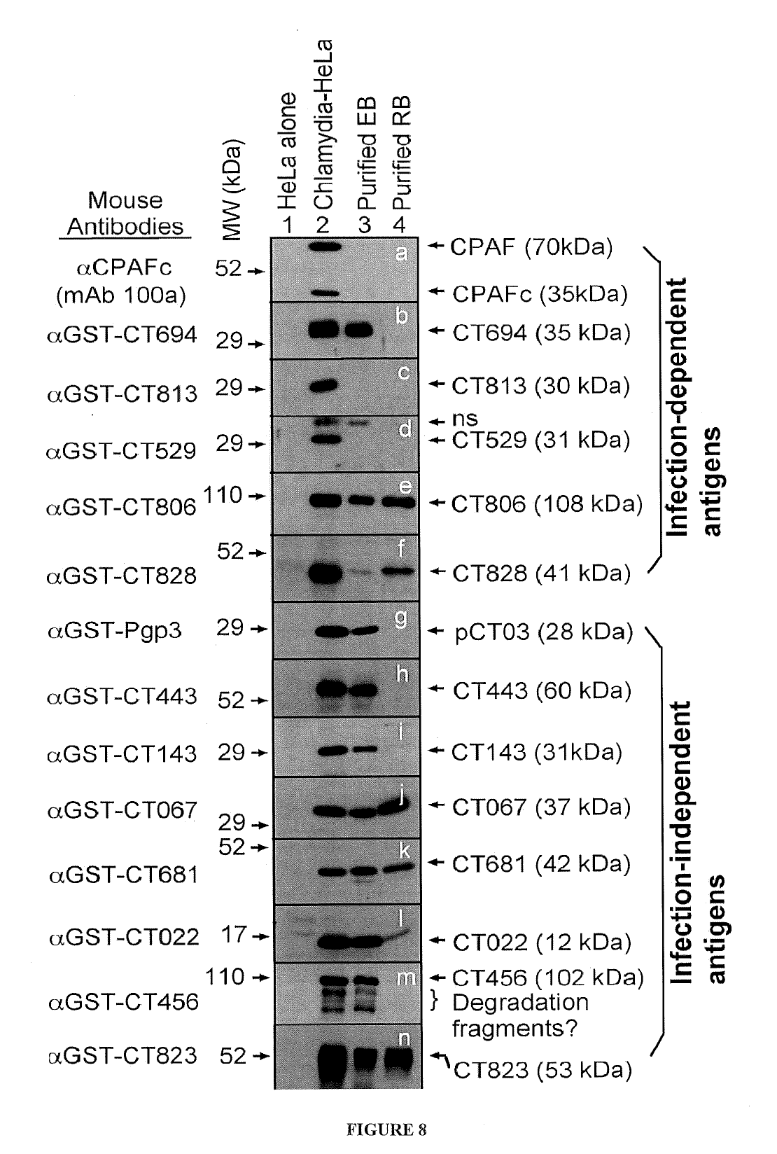

When the 719 human-recognized antigens were resorted based on their detectability by the immunized rabbits and mice (FIG. 7), it was found that 563 of the 719 human-recognized antigens were not detected by either the immunized rabbits or mice, thus classified as infection-dependent antigens, while the remaining 156 are classified as infection-independent antigens. When the levels of endogenous antigens in purified organisms versus infected whole cells were compared, it was found that most of the infection-dependent antigens were significantly enriched during chlamydial intracellular replication in cell culture and many of these antigens were not detectable or only minimally detected in the purified EBs, suggesting that these proteins are produced during live infection but not or only minimally packaged into the infectious EB organisms (FIG. 8). The infection-dependent antigens include proteins secreted to either the inclusion membrane (CT813 and CT529) or host cell cytosol (CT858) during intracellular infection and proteins mainly restricted within the noninfectious RB organisms (CT828). In contrast, most of the infection-independent antigens maintained significant amounts in the purified EB organisms and were not necessarily further enriched during live infection. Many of these antigens are structural proteins of the infectious EB organisms (CT681 & CT443) and/or proteins required for EB invasion of host cells (CT456).

The present study has employed a whole genome scale proteome array consisting of 909 ORFs encoded in C. trachomatis genome and plasmid to profile antibody responses to chlamydial infection in women and identified 719 chlamydial antigens, demonstrating that more than 79% of chlamydial proteins are immunogenic during chlamydial infection in humans. Although there have been many attempts to map C. trachomatis ANTIGENomes using methods such as 2-D gel resolution plus Western blot detection (21), radioimmunoprecipitation plus 2-D gel resolution (22) and partial protein array by drying antigens onto membrane (23), this fusion protein-based whole genome scale proteome array ELISA approach has multiple advantages over the other approaches. First, the amounts of each chlamydial protein are similar in this assay while the amounts of chlamydial proteins used in the traditional 2-D gel approach vary considerably. This is because, in this system, each fusion protein is individually optimized for expression in soluble form and arrayed by saturating the GST binding sites in microplates to ensure that an equal amount of each fusion protein is immobilized to corresponding wells. In contrast, the antigens used in 2-D gels are often derived from either purified chlamydial organisms or infected whole cell lysates, thus the amounts of each protein are determined by ehlamydial biology. The OD values measured in this assay can more accurately reflect the antibody binding intensity while the antibody binding detected in the 2-D gel resolution-based assays can be significantly affected by the varied antigen amounts.

Second, this assay can detect both linear and conformation-dependent antigen/antibody interactions. This is because antigens used in the proteome array were produced in soluble form and assayed in solution and under non-denaturing conditions. The Western blot detection-based assay denatures proteins in SDS and the conventional protein microarray assay requires drying proteins onto membrane that may alter protein conformation. Although this assay system is still based on fusion proteins which cannot always precisely mimic the native conformation of endogenous chlamydial proteins, by assaying interactions of the fusion proteins with human antibodies in solution, this assay system can significantly improve detection sensitivity. For example, among the 27 immunodominant antigens detected in this proteome array assay, 10 were not or only minimally detected in Western blot. The human antibody binding to these conformation-dependent antigens was confirmed by either direct precipitation of the corresponding endogenous chlamydial proteins from Chlamydia-infected cell lysates (FIG. 3) or labeling of the endogenous chlamydial proteins in Chlamydia-infected cells with fusion protein-purified mono-specific human antibodies (FIG. 4).

The finding that 10 out of 27 (37%) immunodominant antigens are conformation-dependant even when pooled polyclonal human antisera were assayed suggests that many important chlamydial antigens are conformation-dependent, which is consistent with the concept that well structured chlamydial proteins are likely presented to the immune system during chlamydial infection in humans. Unfortunately, these chlamydial protein structures may be significantly altered in either Western blot or conventional protein microarray assays and thus may be missed in these assays. The well-studied plasmid-encoded protein Pgp3 is such an example (24). Human antibodies dominantly recognized Pgp3 in ELISA but not Western blot-based assays (25).

Finally, the present approach is comprehensive, covering all proteins that can be expressed. Among all the 919 possible ORFs encoded by the D genome and plasmid, 909 were successfully and only 10 are missing out due to difficulties in expressing soluble proteins. Thus, this whole genome scale array represents 99% coverage assuming all the missing 10 ORFs are real chlamydial proteins. No previous work has ever analyzed chlamydial protein antigenicity on such a grand scale. The significant improvement of this assay system may also contribute to the detection of a higher percentage of chlamydial proteins as antigens.

### Example II

### Protection and Pathology Studies on Immunodominant Proteins

Having determined that the proteins described herein are dominantly recognized by antisera from patients infected with Chlamydia, further studies were carried out to evaluate whether these proteins can also induce protective immunity against chlamydial challenge infection in mice and/or reduce oviduct pathology (hydrosalpinx).

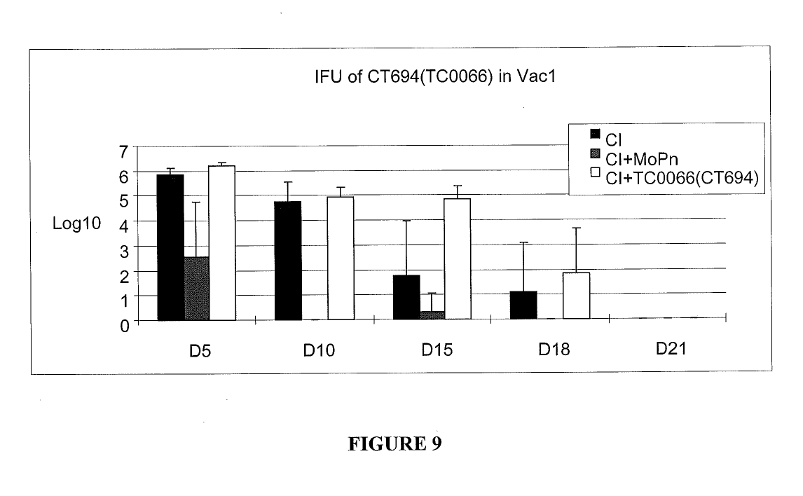

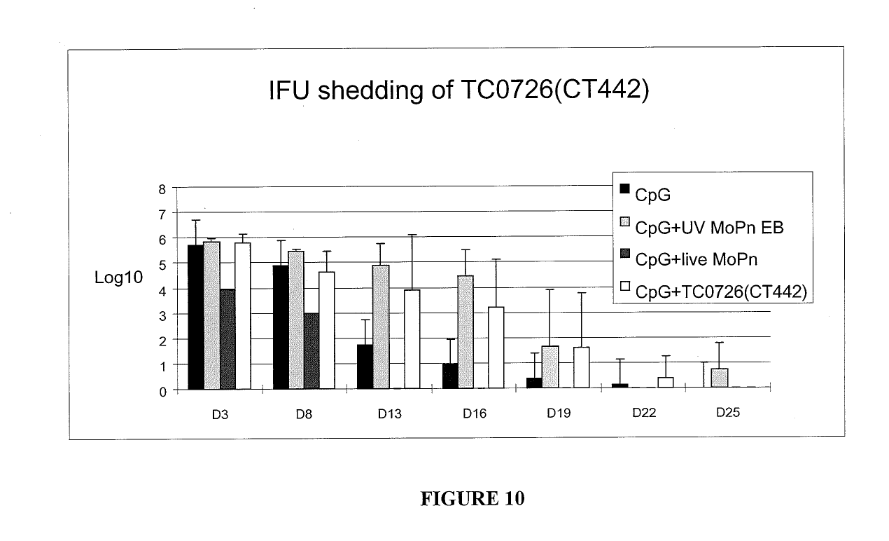

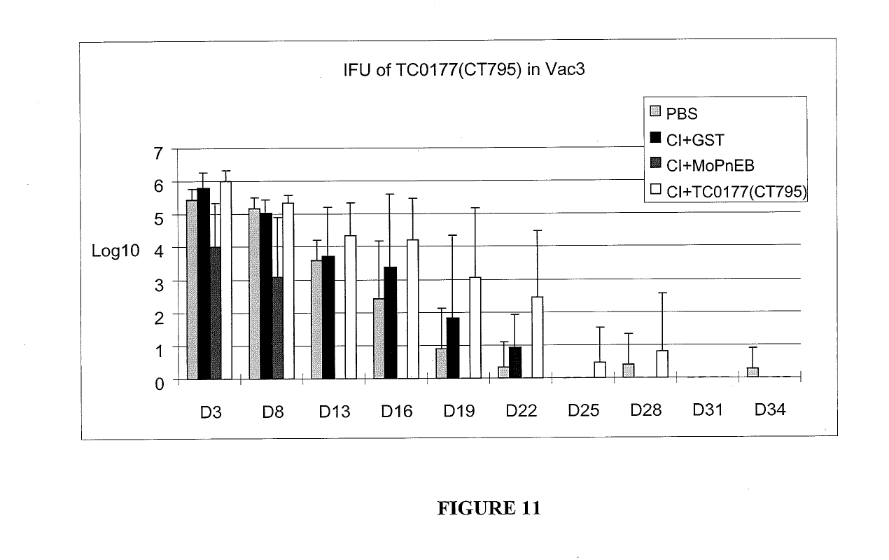

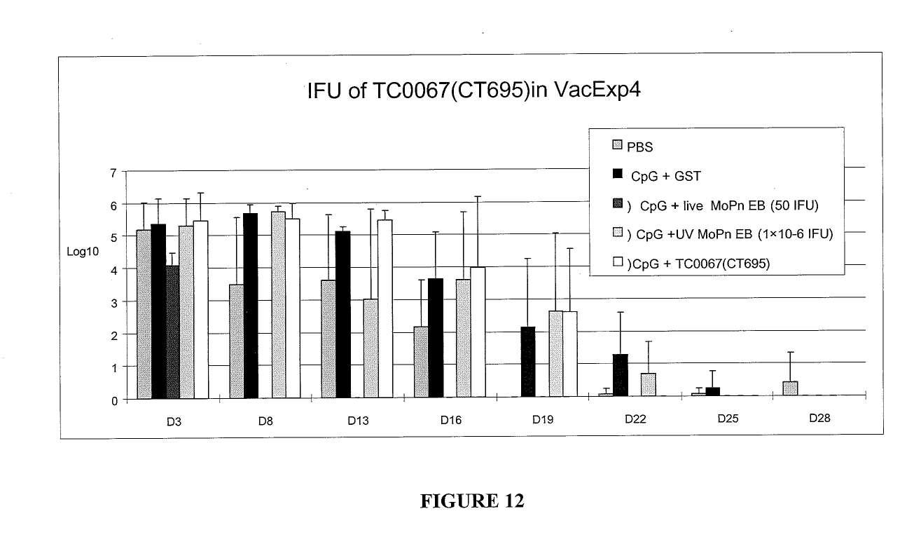

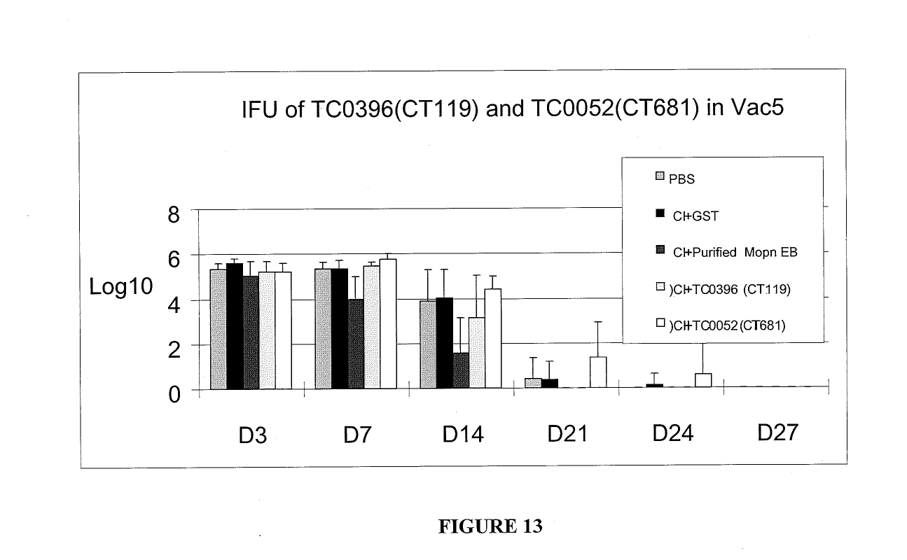

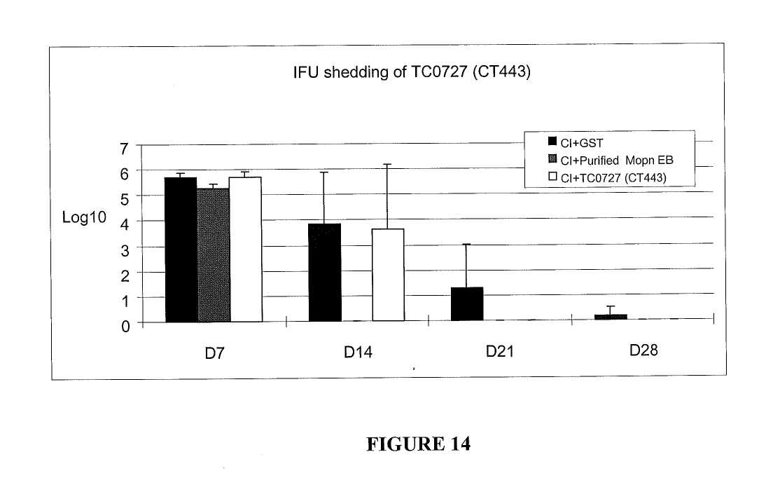

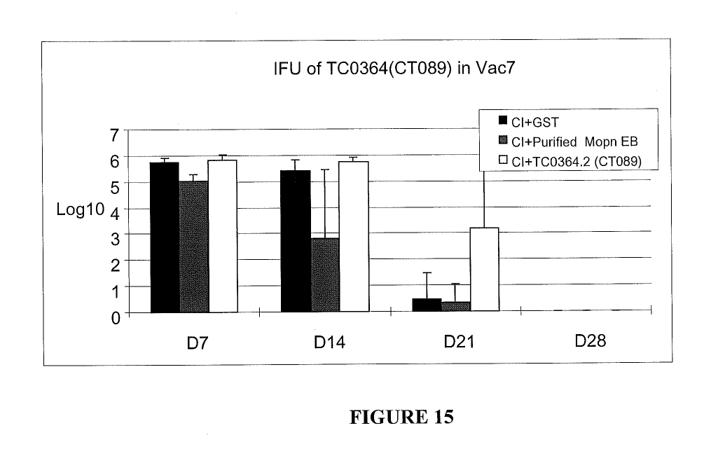

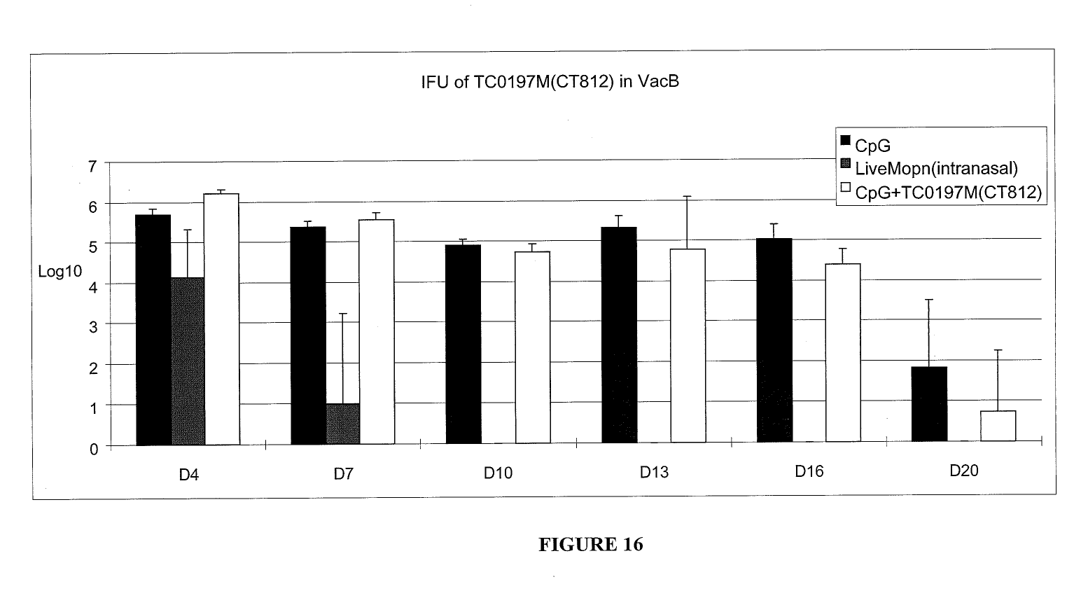

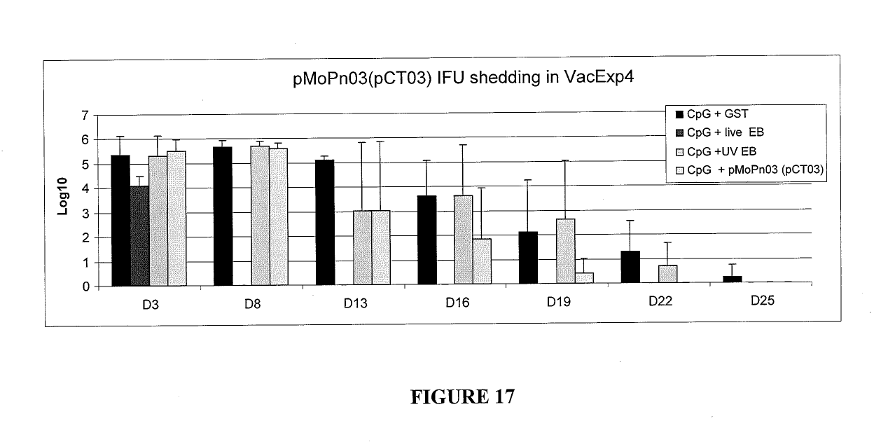

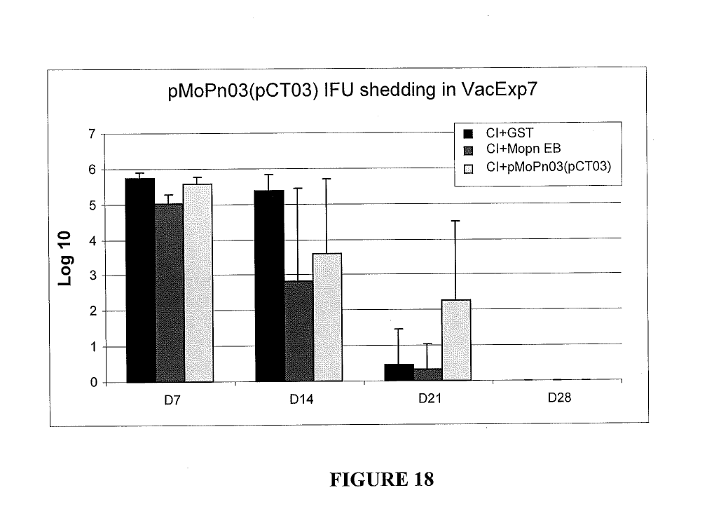

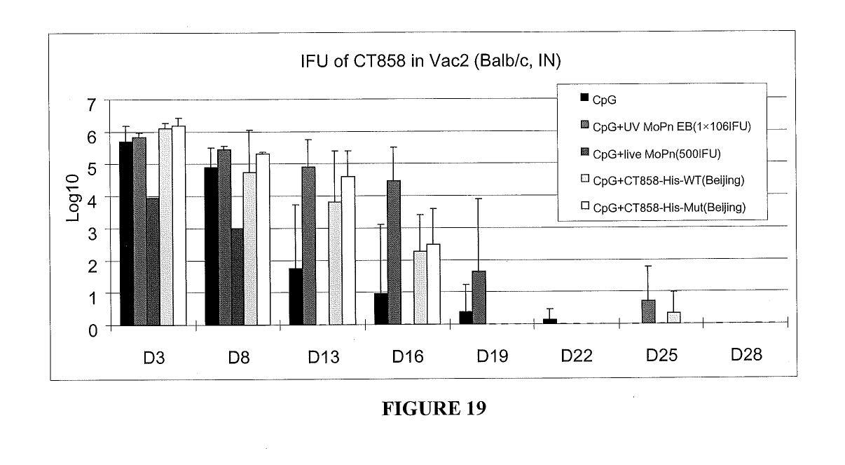

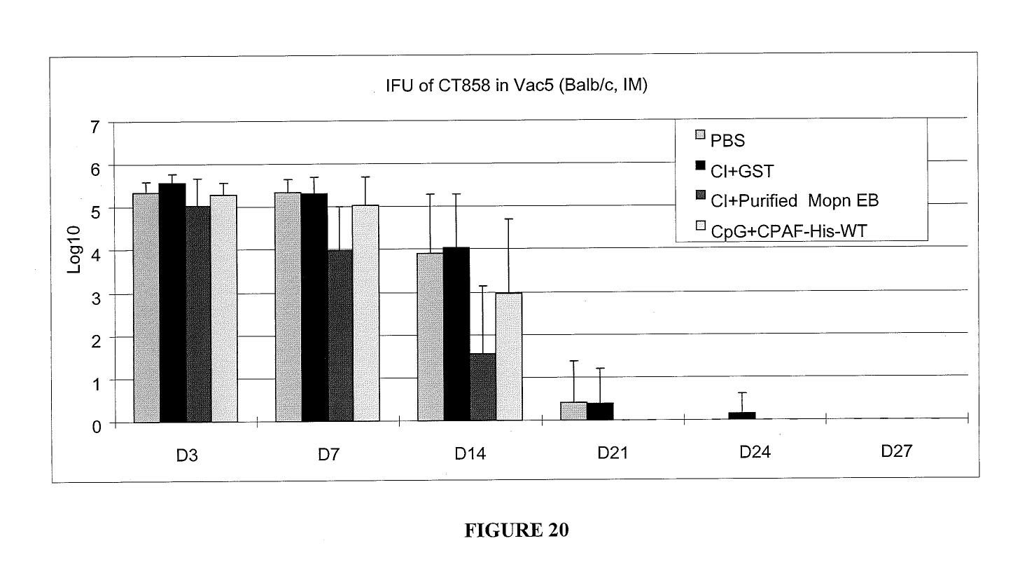

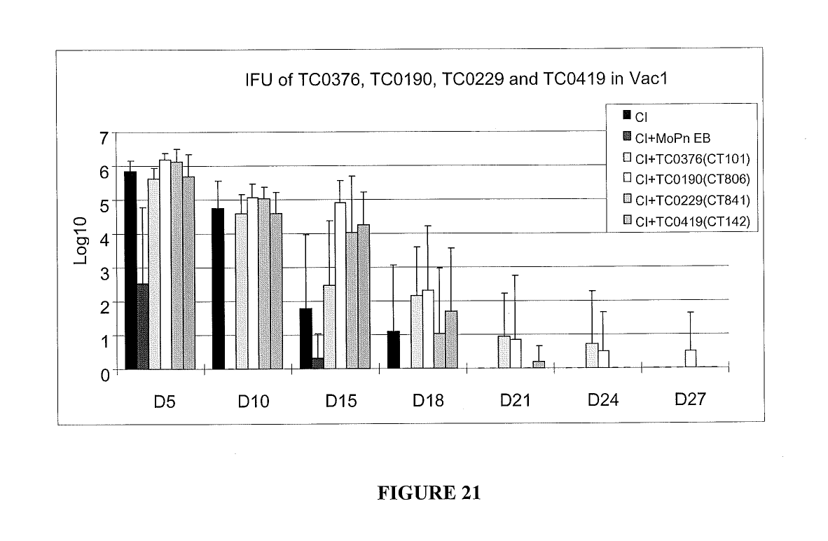

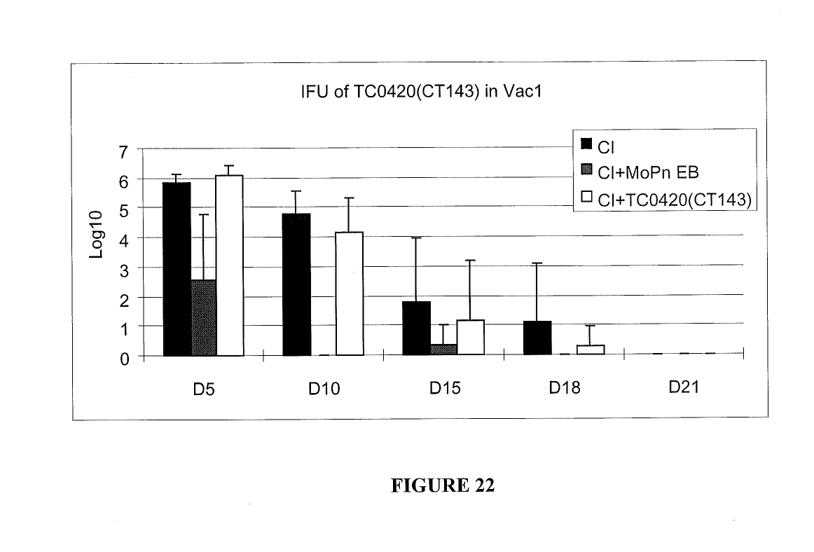

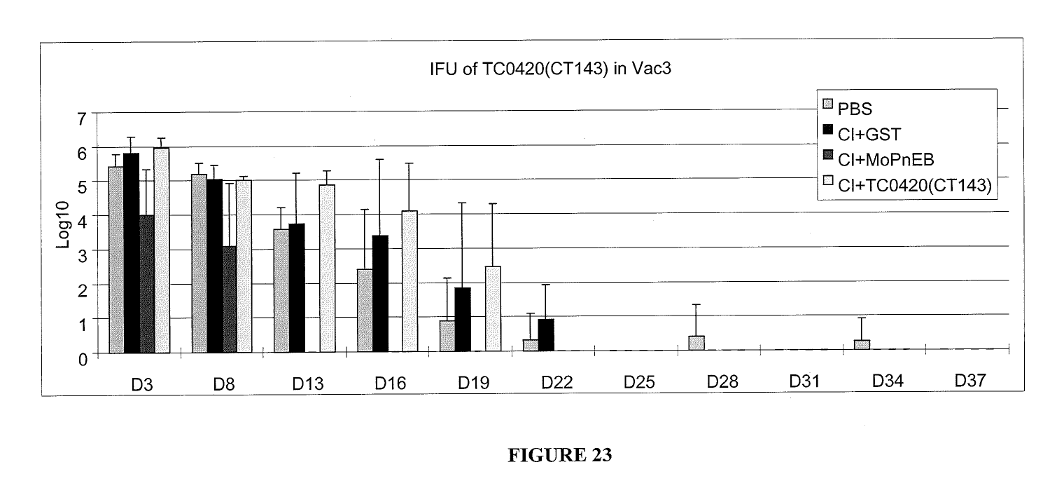

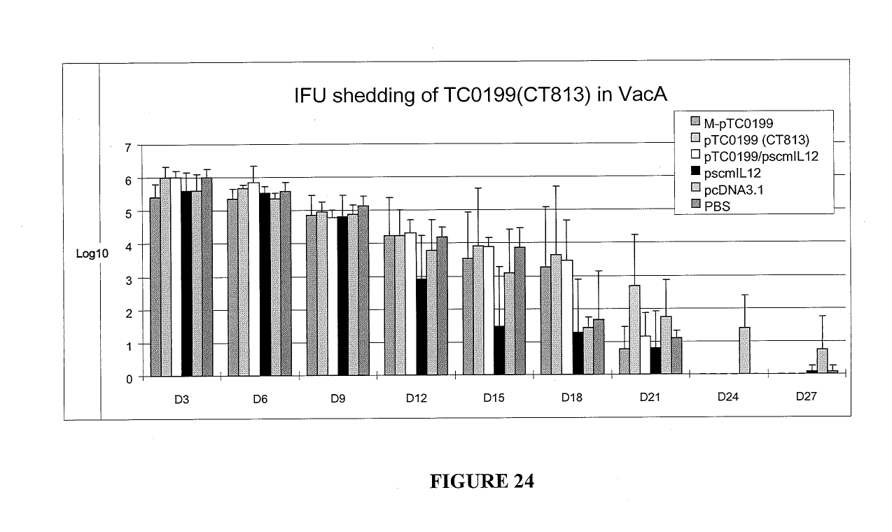

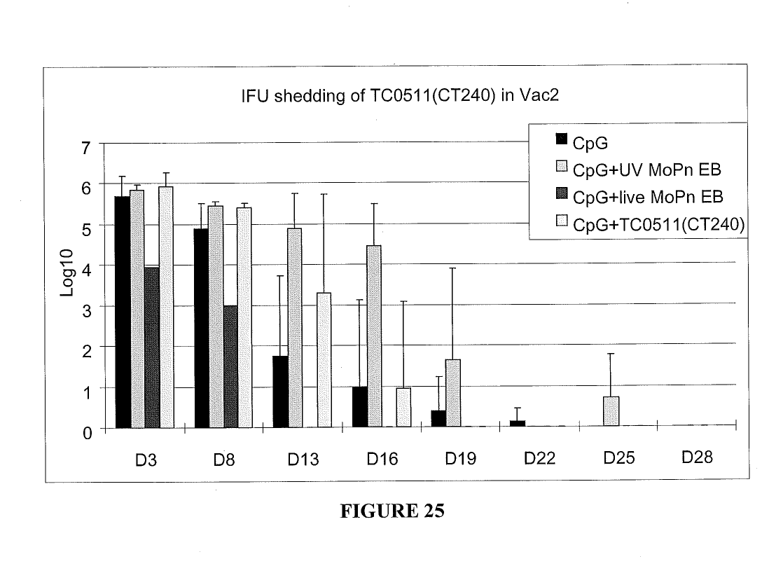

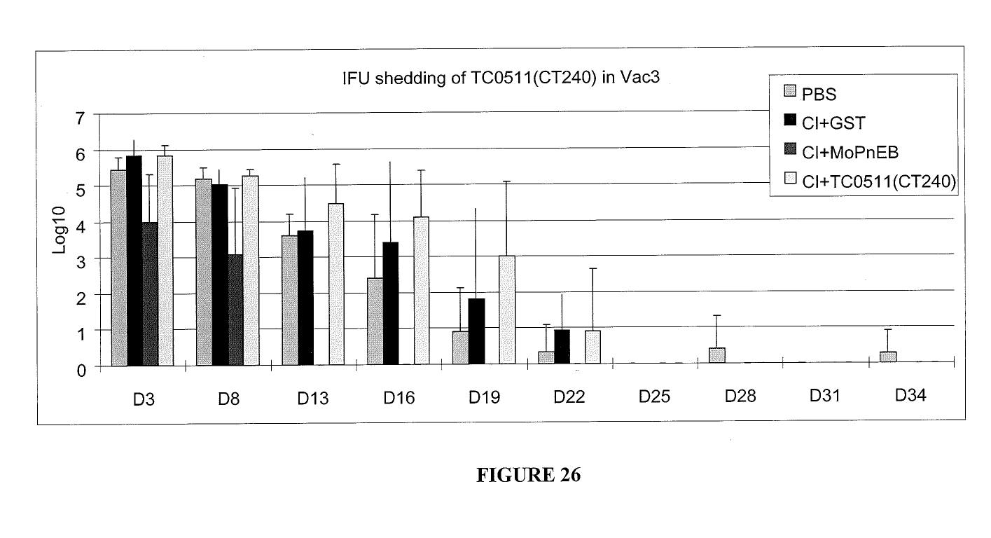

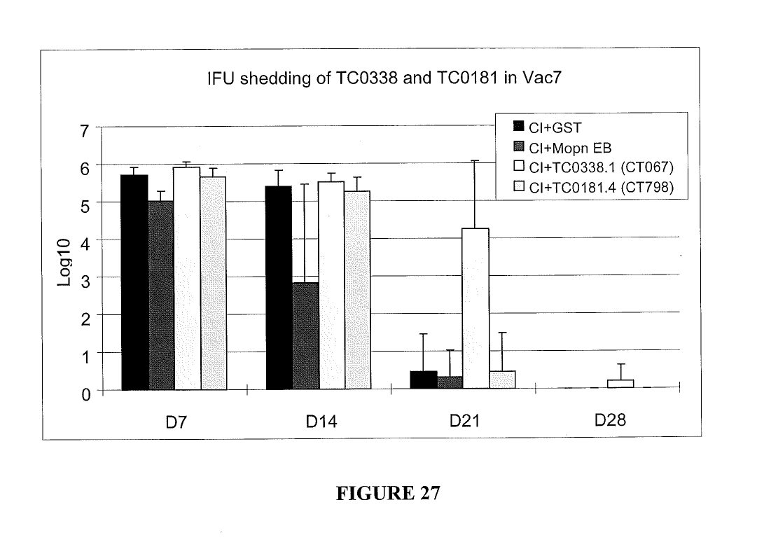

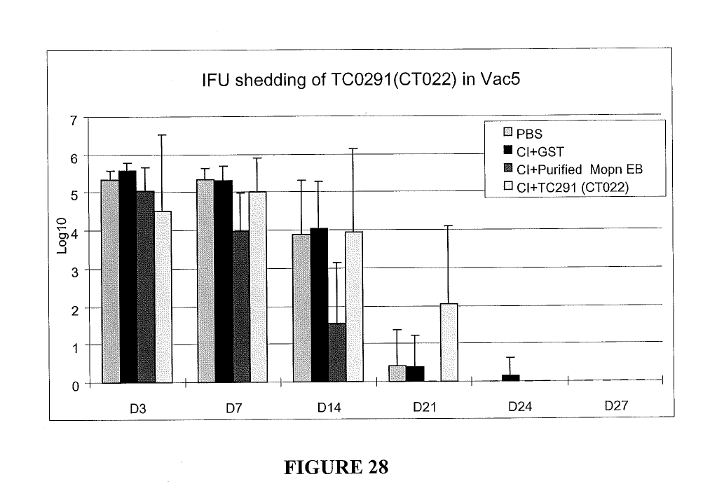

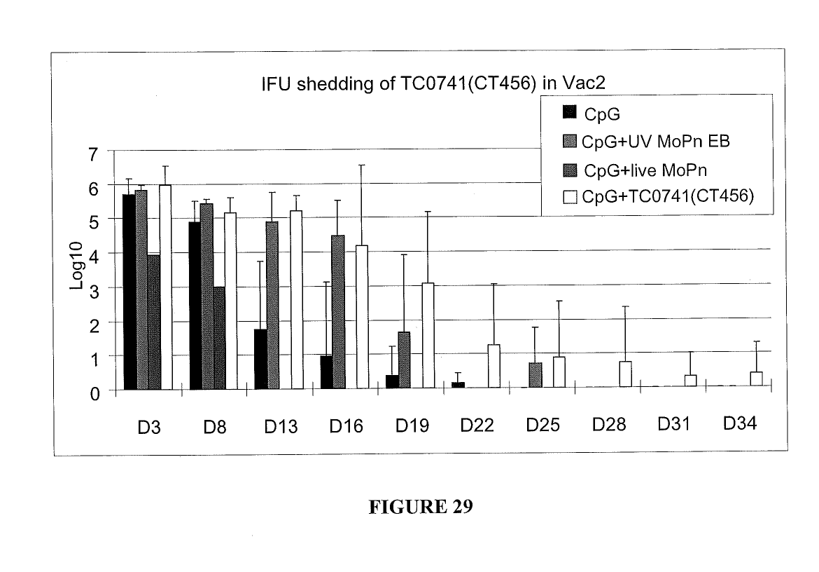

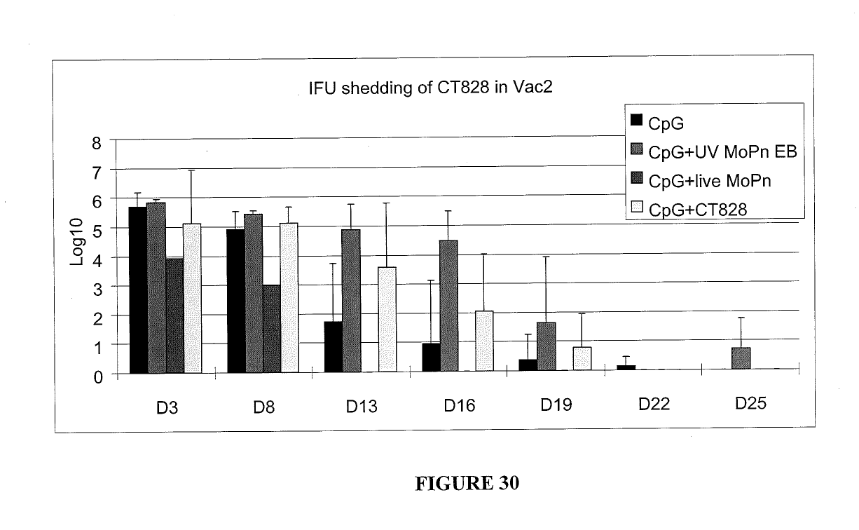

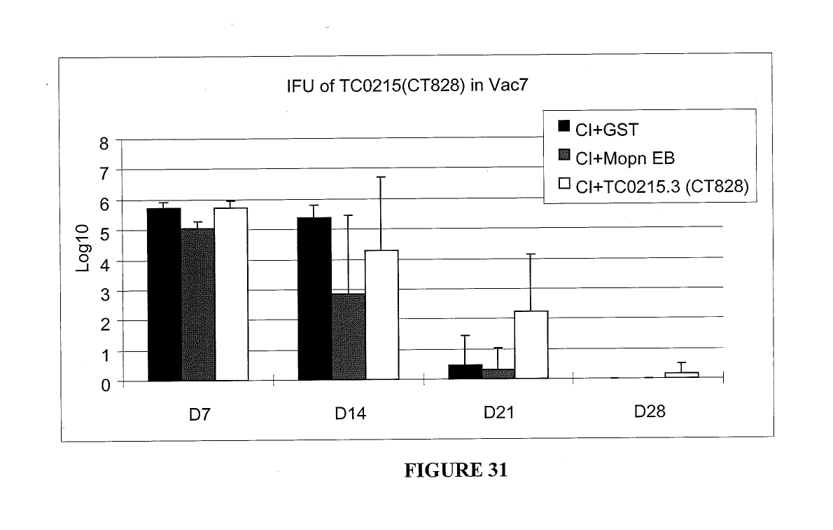

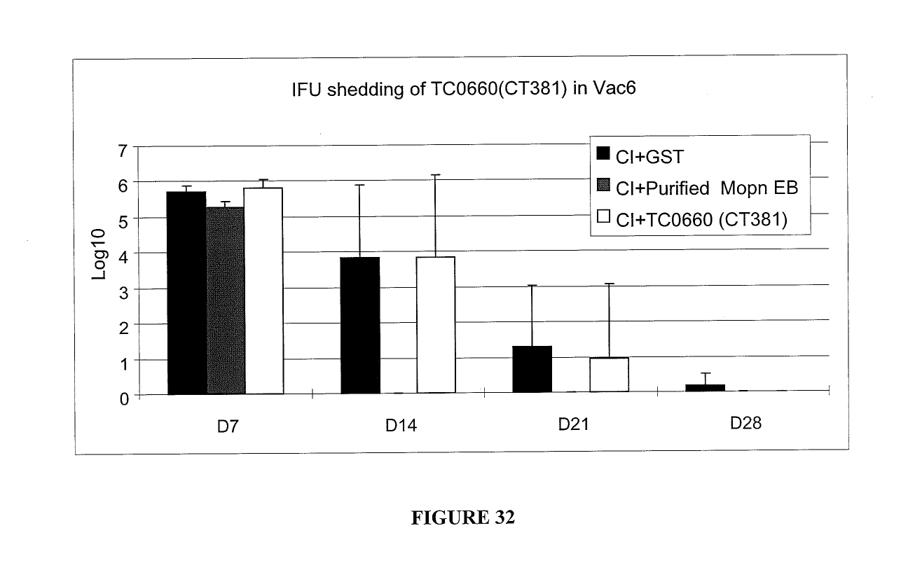

Balb/c female mice were immunized with the corresponding purified chlamydial antigens plus CpG adjuvant for intranasal immunization (IN) [Table 2 and FIG. 10 (CT442), Table 4 and FIG. 12 (CT695), Table 8 and FIG. 16 (CT812), Table 9 and FIG. 17 (pCT03), Table 11 and FIG. 19 (CT858), Table 16 and FIG. 24 (CT813), Table 17 and FIG. 25 (CT240), Table 21 and FIG. 29 (CT456) and Table 22 and FIG. 30 (CT828)]. Purified chlamydial antigens plus CpG adjuvant were in incomplete Freund's adjuvant (WA) for intramuscular injection (IM) [Table 1 and FIG. 9 (CT694), Table 3 and FIG. 11 (CT795), Table 5 and FIG. 13 (CT119 and CT681), Table 6 and FIG. 14 (CT443), Table 7 and FIG. 15 (CT089), Table 10 and FIG. 18 (pCT03), Table 12 and FIG. 20 (CT858), Table 13 and FIG. 21 (CT841, CT101, CT142, CT806), Table 14 and FIG. 22 (CT143), Table 15 and FIG. 23 (CT143), Table 18 and FIG. 26 (CT240), Table 19 and FIG. 27 (CT798, CT067), Table 20 and FIG. 28 (CT022), Table 23 and FIG. 31 (CT828), Table 24 and FIG. 32 (CT381)].

Mice were immunized three times each with 20 to 200 ug of a given antigen with 10 ug of CpG and equal volume of IFA (IFA for IM only). Thirty days after the last injection, mice were challenged intravaginally with live MoPn organisms. On different days (D) after infection, vaginal swabs were taken for monitoring the shedding of live organisms. On days 60 to 80 post infection, all mice were sacrificed for visualizing oviduct pathologies (visible hydrosalpinx). The live organism recovery yield was expressed as Log¹⁰ IFUs and the hydrosalpinx pathology as incidence and severity scores. Two control groups were included in each experiment, with CpG+GST immunization for negative control and CpG+MoPn EB as positive control.

The foregoing is illustrative of the present invention, and is not to be construed as limiting thereof. The invention is defined by the following claims, with equivalents of the claims to be included therein. All publications, patent applications, patents, patent publications, sequences (nucleotide sequences, single polymorphism nucleotides, amino acid sequences, etc.) identified in the GenBank® database or other sequence databases according to the accession numbers provided herein, and any other references cited herein are incorporated by reference in their entireties for the teachings relevant to the sentence and/or paragraph in which the reference is presented.

### REFERENCES

1. Tang, F. F., H. L. Chang, Y. T. Huang, and K. C. Wang. 1957. Studies on the etiology of trachoma with special reference to isolation of the virus in chick embryo. Chin Med J 75:429-447.
2. Schachter, J., E. C. Hill, E. B. King, V. R. Coleman, P. Jones, and K. F. Meyer. 1975. Chlamydial infection in women with cervical dysplasia. Am J Obstet Gynecol 123:753-757.
3. Sherman, K. J., J. R. Daling, A. Stergachis, N. S. Weiss, H. M, Foy, S. P. Wang, and J. T. Grayston. 1990. Sexually transmitted diseases and tubal pregnancy. Sex Transm Dis 17:115-121.
4. Kinnunen, A. H., H. M. Surcel, M. Lehtinen, J. Karhukorpi, A. Tiitinen, M. Halttunen, A. Bloigu, R. P. Morrison, R. Karttunen, and J. Paavonen. 2002. HLA DQ alleles and interleukin-10 polymorphism associated with Chlamydia trachomatis-related tubal factor infertility: a case-control study. Hum Reprod 17:2073-2078.
5. Grayston, J. T., R. L. Woolridge, and S. Wang. 1962. Trachoma vaccine studies on Taiwan. Ann N Y Acad Sci 98:352-367.
6. Rasmussen, S. J., L. Eckmann, A. J. Quayle, L. Shen, Y. X. Zhang, D. J. Anderson, J. Fierer, R. S. Stephens, and M. F. Kagnoff. 1997. Secretion of proinflammatory cytokines by epithelial cells in response to Chlamydia infection suggests a central role for epithelial cells in chlamydial pathogenesis. J Clin Invest 99:77-87.
7. Cheng, W., P. Shivshankar, Y. Zhong, D. Chen, Z. Li, and G. Zhong. 2008. Intracellular interleukin-1alpha mediates interleukin-8 production induced by Chlamydia trachomatis infection via a mechanism independent of type I interleukin-1 receptor. Infect Immun 76:942-951.
8. Stephens, R. S. 2003. The cellular paradigm of chlamydial pathogenesis. Trends Microbiol 11:44-51.
9. Zhong, G. 2009. Killing me softly: chlamydial use of proteolysis for evading host defenses. Trends Microbial 17:467-474.
10. Hackstadt, T. 1998. The diverse habitats of obligate intracellular parasites. Curr Opin Microbial 1:82-87.
11. Hackstadt, T., E. R. Fischer, M. A. Scidmore, D. D. Rockey, and R. A. Heinzen. 1997. Origins and functions of the chlamydial inclusion. Trends Microbial 5:288-293.
12. Jorgensen, I., and R. H. Valdivia. 2008. Pmp-like proteins Pls1 and Pls2 are secreted into the lumen of the Chlamydia trachomatis inclusion. Infect Immun 76:3940-3950.
13. Rockey, D. D., D. Grosenbach, D. E. Hruby, M. G. Peacock, R. A. Heinzen, and T. Hackstadt. 1997. Chlamydia psittaci IncA is phosphorylated by the host cell and is exposed on the cytoplasmic face of the developing inclusion. Mol Microbiol 24:217-228.
14. Scidmore, M. A., and T. Hackstadt. 2001. Mammalian 14-3-3beta associates with the Chlamydia trachomatis inclusion membrane via its interaction with IncG. Mol Microbiol 39:1638-1650.
15. Hackstadt, T., M. A. Scidmore-Carlson, E. I. Shaw, and E. R. Fischer. 1999. The Chlamydia trachomatis IncA protein is required for homotypic vesicle fusion. Cell Microbiol 1:119-130.
16. Li, Z., C. Chen, D. Chen, Y. Wu, Y. Zhong, and G. Zhong. 2008. Characterization of fifty putative inclusion membrane proteins encoded in the Chlamydia trachomatis genome. Infect Immun 76:2746-2757.
17. Zhong, G., P. Fan, H. Ji, F. Dong, and Y. Huang. 2001. Identification of a chlamydial protease-like activity factor responsible for the degradation of host transcription factors. J Exp Med 193:935-942.
18. Clifton, D. R., K. A. Fields, S. S. Grieshaber, C. A. Dooley, E. R. Fischer, D. J. Mead, R. A. Carabeo, and T. Hackstadt. 2004. A chlamydial type III translocated protein is tyrosine-phosphorylated at the site of entry and associated with recruitment of actin. Proc Natl Acad Sci USA 101:10166-10171.
19. Sharma, J., A. M. Bosnic, J. M. Piper, and G. Zhong. 2004. Human antibody responses to a Chlamydia-secreted protease factor. Infect Immun 72:7164-7171.
20. Murthy, A K., J. P. Chambers, P. A. Meier, G. Zhong, and B. P. Arulanandam. 2007. Intranasal vaccination with a secreted chlamydial protein enhances resolution of genital Chlamydia muridarum infection, protects against oviduct pathology, and is highly dependent upon endogenous gamma interferon production. Infect Immun 75:666-676.
21. Caldwell, H. D., C. C. Kuo, and G. E. Kenny. 1975. Antigenic analysis of Chlamydiae by two-dimensional immunoelectrophoresis. II. A trachoma-LGV-specific antigen. J Immunol 115:969-975.
22. Shaw, A. C., B. B. Vandahl, M. R. Larsen, P. Roepstorff, K. Gevaert, J. Vandekerckhove, G. Christiansen, and S. Birkelund. 2002. Characterization of a secreted Chlamydia protease. Cell Microbial 4:411-424.
23. Molina, D. M., S. Pal, M. A. Kayala, A. Teng, R. I. Kim, P. Baldi, P. L. Feigner, X. Liang, and L. M. de la Maza. 2009. Identification of immunodominant antigens of Chlamydia trachomatis using proteome microarrays. Vaccine Epub Dec. 29, 2009.
24. Li, Z., Y. Zhong, L. Lei, Y. Wu, S. Wang, and G. Zhong. 2008. Antibodies from women urogenitally infected with C. trachomatis predominantly recognized the plasmid protein pgp3 in a conformation-dependent manner. BMC Microbial 8:90.
25. Comanducci, M., R. Cevenini, A. Moroni, M. M. Giuliani, S. Ricci, V. Scarlato, and G. Ratti. 1993. Expression of a plasmid gene of Chlamydia trachomatis encoding a novel 28 kDa antigen. J Gen Microbial 139:1083-1092.
26. Murthy, A. K., Y. Cong, C. Murphey, M. N. Guentzel, T. G. Forsthuber, G. Zhong, and B. P. Arulanandam. 2006, Chlamydial protease-like activity factor induces protective immunity against genital chlamydial infection in transgenic mice that express the human HLA-DR4 allele. Infect Immun 74:6722-6729.
27. Brunham, R. C., and R. W. Peeling. 1994. Chlamydia trachomatis antigens: role in immunity and pathogenesis. Infect Agents Dis 3:218-233.
28. Peeling, R. W., J. Kimani, F. Plummer, I. Maclean, M. Cheang, J. Bwayo, and R. C. Brunham. 1997. Antibody to chlamydial hsp60 predicts an increased risk for chlamydial pelvic inflammatory disease. J Infect Dis 175:1153-1158.
29. Yi, Y., X. Yang, and R. C. Brunham. 1997. Autoimmunity to heat shock protein 60 and antigen-specific production of interleukin-10. Infect Immun 65:1669-1674.
30. LaVerda, D., L. N. Albanese, P. E. Ruther, S. G. Morrison, R. P. Morrison, K. A. Ault, and G. I. Byrne. 2000. Seroreactivity to Chlamydia trachomatis Hsp10 correlates with severity of human genital tract disease. Infect Immun 68:303-309.
31. Rockey, D. D., J. Wang, L. Lei, and G. Zhong. 2009. Chlamydia vaccine candidates and tools for chlamydial antigen discovery. Expert Rev Vaccines 8:1365-1377.
32. Greene, W., Y. Xiao, Y. Huang, G. McClarty, and G. Zhong. 2004. Chlamydia-infected cells continue to undergo mitosis and resist induction of apoptosis. Infect Immun 72:451-460.
33. Sharma, J., Y. Zhong, F. Dong, J. M. Piper, G. Wang, and G. Zhong. 2006. Profiling of human antibody responses to Chlamydia trachomatis urogenital tract infection using microplates arrayed with 156 chlamydial fusion proteins. Infect Immun 74:1490-1499.
34. Wang, J., L. Chen, F. Chen, X. Zhang, Y. Zhang, J. Baseman, S. Perdue, I. T. Yeh, R. Sharan, M. Holland, R. Bailey, D. Mabey, P. Yu, and G. Zhong. 2009. A chlamydial type III-secreted effector protein (Tarp) is predominantly recognized by antibodies from humans infected with Chlamydia trachomatis and induces protective immunity against upper genital tract pathologies in mice. Vaccine 27:2967-2980.
35. Stephens, R. S., S. Kalman, C. Lammel, J. Fan, R. Marathe, L. Aravind, W. Mitchell, L. Olinger, R. L. Tatusov, Q. Zhao, E. V. Koonin, and R. W. Davis. 1998. Genome sequence of an obligate intracellular pathogen of humans: Chlamydia trachomatis, Science 282:754-759.
36. Jensen, K. T., L. Petersen, S. Falk, P. Iversen, P. Andersen, M. Theisen, and A. Krogh. 2006. Novel overlapping coding sequences in Chlamydia trachomatis. FEMS Microbiol Lett 265:106-117.
37. Zhong, G., I. Toth, R. Reid, and R. C. Brunham. 1993. Immunogenicity evaluation of a lipidic amino acid-based synthetic peptide vaccine for Chlamydia trachomatis. J Immunol 151:3728-3736.
38. Zhong, G., C. Reis e Sousa, and R. N. Germain. 1997. Production, specificity, and functionality of monoclonal antibodies to specific peptide-major histocompatibility complex class II complexes formed by processing of exogenous protein. Proc Natl Acad Sci USA 94:13856-13861.
39. Zhong, G., J. Berry, and R. C. Brunham. 1994. Antibody recognition of a neutralization epitope on the major outer membrane protein of Chlamydia trachomatis. Infect Immun 62:1576-1583.
40. Zhong, G., and R. C. Brunham. 1992. Antibody responses to the chlamydial heat shock proteins hsp60 and hsp70 are H-2 linked. Infect Immun 60:3143-3149.
41. Zhong, G. M., and R. C. Brunham. 1991. Antigenic determinants of the chlamydial major outer membrane protein resolved at a single amino acid level. Infect Immun 59:1141-1147.
42, Zhong, G., F. Castellino, P. Romagnoli, and R. N. Germain. 1996. Evidence that binding site occupancy is necessary and sufficient for effective major histocompatibility complex (MHC) class II transport through the secretory pathway redefines the primary function of class II-associated invariant chain peptides (CLIP). J Exp Med 184:2061-2066.
43. Fan, T., H. Lu, H. Hu, L. Shi, G. A. McClarty, D. M. Nance, A. H. Greenberg, and G. Zhong. 1998. Inhibition of apoptosis in Chlamydia-infected cells: blockade of mitochondrial cytochrome c release and caspase activation. J Exp Med 187:487-496.
44. Sharma, J., F. Dong, M. Pirbhai, and G. Zhong. 2005. Inhibition of proteolytic activity of a chlamydial proteasome/protease-like activity factor by antibodies from humans infected with Chlamydia trachomatis. Infect Immun 73:4414-4419.
45. Dong, F., Y. Zhong, B. Arulanandam, and G. Thong. 2005. Production of a proteolytically active protein, chlamydial protease/proteasome-like activity factor, by five different Chlamydia species. Infect Immun 73:1868-1872.
46. Dong, F., M. Pirbhai, Y. Xiao, Y. Zhong, Y. Wu, and G. Zhong. 2005. Degradation of the proapoptotic proteins Bik, Puma, and Bim with Bcl-2 domain 3 homology in Chlamydia trachomatis-infected cells. Infect Immun 73:1861-1864.

TABLE 1
VacExp1 Balb/c, IM

| | | | | | | | | | | | Oviduct Pathology | | | |
|---|---|---|---|---|---|---|---|---|---|---|---|---|---|---|
| | | | | | | | | | | | Hydrosalpinx | | | |
| | | IFU(Log 10) | | | | | | | | | Incidence | | | Severity |
| | | D3 | D8 | D13 | D16 | D19 | D22 | D25 | D28 | D31 | Uni- | Bi- | Total | Score |
| CI | X | 5.85 | 4.76 | 1.11 | 0 | 0 | 0 | 0 | 0 | 0 | 1/5 | 2/5 | 3/5 | 1.2 |
| | SD | 0.29 | 0.80 | 1.96 | 0 | 0 | 0 | 0 | 0 | 0 | | | | 1.10 |
| CI + MoPn | X | 2.54 | 0 | 0 | 0 | 0 | 0 | 0 | 0 | 0 | 2/5 | 0/5 | 2/5 | 0.4 |
| | SD | 2.23 | 0 | 0 | 0 | 0 | 0 | 0 | 0 | 0 | | | | 0.55 |
| CI + TC0066*CT694) | X | 6.19 | 4.95 | 1.84 | 0 | 0 | 0 | 0 | 0 | 0 | 1/4 | 2/4 | 3/4 | 2 |
| | SD | 0.15 | 0.40 | 1.81 | 0 | 0 | 0 | 0 | 0 | 0 | | | | 1.41 |

Note:
CI = CpG + IFA
X = mean
SD = Standard deviations

TABLE 2
VacExp2 - Balb/c, IN

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | Severity |
| | | D3 | D8 | D13 | D16 | D19 | D22 | D25 | D28 | D31 | D34 | Uni- | Bi- | Total | Score |
| CpG+ | X | 5.69 | 4.90 | 1.74 | 0.96 | 0.38 | 0.14 | 0 | 0 | 0 | 0 | 2/5 | 1/5 | 3/5 | 2 |
| | SD | 0.48 | 0.61 | 1.99 | 2.15 | 0.85 | 0.31 | 0 | 0 | 0 | 0 | | | | 2 |
| CpG + UV MoPn EB_{(1 × 10}⁶ _{IFU)} | X | 5.83 | 5.44 | 4.89 | 4.47 | 1.64 | 0 | 0.7 | 0 | 0 | 0 | 2/5 | 2/5 | 4/5 | 2.2 |
| | SD | 0.13 | 0.12 | 0.85 | 1.02 | 2.25 | 0 | 1.1 | 0 | 0 | 0 | | | | 1.48 |
| CpG + live MoPn_{(500 IFU)} | X | 3.94 | 3 | 0 | 0 | 0 | 0 | 0 | 0 | 0 | 0 | 0 | 0 | 0/1 | 0 |
| | SD | 0 | 0 | 0 | 0 | 0 | 0 | 0 | 0 | 0 | 0 | | | | 0 |
| CpG + TC0726_{(CT442)} | X | 5.81 | 4.62 | 3.89 | 3.20 | 1.58 | 0.39 | 0 | 0 | 0 | 0 | 1/5 | 4/5 | 5/5 | 3.8 |
| | SD | 0.35 | 0.84 | 2.22 | 1.90 | 2.18 | 0.86 | 0 | 0 | 0 | 0 | | | | 1.30 |

TABLE 3
Vaccine Experiment 3 - Balb/c, IM

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | Severity |
| | | D3 | D8 | D13 | D16 | D19 | D22 | D25 | D28 | D31 | D34 | Uni- | Bi- | Total | Score |
| PBS | X | 5.44 | 5.18 | 3.60 | 2.41 | 0.90 | 0.34 | 0 | 0.41 | 0 | 0.28 | 2/5 | 1/5 | 3/5 | 2.8 |
| | SD | 0.35 | 0.33 | 0.61 | 1.75 | 1.23 | 0.76 | 0 | 0.91 | 0 | 0.63 | | | | 2.95 |
| CI + GST | X | 5.82 | 5.03 | 3.73 | 3.38 | 1.82 | 0.93 | 0 | 0 | 0 | 0 | 1/5 | 2/5 | 3/5 | 4 |
| | SD | 0.46 | 0.41 | 1.49 | 2.23 | 2.51 | 0.99 | 0 | 0 | 0 | 0 | | | | 4 |
| CI + MoPn EB_{(1 × 10}⁵ _{IFU)} | X | 4.00 | 3.10 | 0 | 0 | 0 | 0 | 0 | 0 | 0 | 0 | 1/5 | 2/5 | 3/5 | 1.4 |
| | SD | 1.32 | 1.82 | 0 | 0 | 0 | 0 | 0 | 0 | 0 | 0 | | | | 1.67 |
| CI + TC0177_{(CT795)} | X | 5.99 | 5.34 | 4.35 | 4.20 | 3.06 | 2.47 | 0.47 | 0.79 | 0 | 0 | 1/5 | 4/5 | 5/5 | 3.6 |
| | SD | 0.34 | 0.24 | 1.01 | 1.27 | 2.11 | 2.02 | 1.06 | 1.76 | 0 | 0 | | | | 2.07 |

TABLE 4
VacExp4 - IN

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | Severity |
| | | D3 | D8 | D13 | D16 | D19 | D22 | D25 | D28 | D31 | D34 | Uni- | Bi- | Total | Score |
| PBS | X | 5.18 | 3.49 | 3.61 | 2.16 | 0 | 0.08 | 0.08 | 0.41 | 0 | 0 | 2/5 | 2/5 | 4/5 | 2.4 |
| | SD | 0.84 | 2.08 | 2.02 | 1.46 | 0 | 0.18 | 0.18 | 0.92 | 0 | 0 | | | | 2.41 |
| CpG + GST | X | 5.35 | 5.67 | 5.11 | 3.62 | 2.14 | 1.28 | 0.24 | 0 | 0 | 0 | 1/5 | 4/5 | 5/5 | 5 |
| | SD | 0.79 | 0.26 | 0.15 | 1.45 | 2.12 | 1.29 | .053 | 0 | 0 | 0 | | | | 2 |
| CpG + live MoPn EB (50 IFU) | X | 4.07 | 0 | 0 | 0 | 0 | 0 | 0 | 0 | 0 | 0 | 0/5 | 1/5 | 1/5 | 0.4 |
| | SD | 0.40 | 0 | 0 | 0 | 0 | 0 | 0 | 0 | 0 | 0 | | | | 0.89 |
| CpG + UV MoPn EB (1 × 10−6 IFU) | X | 5.31 | 5.71 | 3.03 | 3.61 | 2.62 | 0.69 | 0 | 0 | 0 | 0 | 1/5 | 4/5 | 5/5 | 5.2 |
| | SD | 0.83 | 0.18 | 2.78 | 2.09 | 2.42 | 0.98 | 0 | 0 | 0 | 0 | | | | 3.03 |
| CpG + TC0067 (CT695) | X | 5.45 | 5.49 | 5.44 | 3.98 | 2.61 | 0 | 0 | 0 | 0 | 0 | 2/4 | 2/4 | 4/4 | 3.25 |
| | SD | 0.86 | 0.48 | 0.31 | 2.19 | 1.94 | 0 | 0 | 0 | 0 | 0 | | | | 3.20 |

TABLE 5
VacExp5 - Balb/c, IM

| | | | | | | | | Oviduct Pathology | | | |
|---|---|---|---|---|---|---|---|---|---|---|---|
| | | | | | | | | Hydrosalpinx | | | |
| | | IFU(Log 10) | | | | | | Incidence | | | Severity |
| | | D3 | D7 | D14 | D21 | D24 | D27 | Uni- | Bi- | Total | Score |
| PBS | X | 5.34 | 5.34 | 3.90 | 0.41 | 0 | 0 | 4/10 | 0/10 | 4/10 | 0.9 |
| | SD | 0.25 | 0.31 | 1.40 | 0.96 | 0 | 0 | | | | 1.20 |
| CI + GST | X | 5.57 | 5.32 | 4.03 | 0.37 | 0.15 | 0 | 2/10 | 2/10 | 4/10 | 1.9 |
| | SD | 0.22 | 0.38 | 1.26 | 0.83 | 0.47 | 0 | | | | 2.51 |
| CI + Purified Mopn EB | X | 5.04 | 3.99 | 1.55 | 0 | 0 | 0 | 2/10 | 0/10 | 2/10 | 0.6 |
| | SD | 0.63 | 1.01 | 1.60 | 0 | 0 | 0 | | | | 1.35 |
| CI + TC0396 (CT119) | X | 5.20 | 5.47 | 3.12 | 0 | 0 | 0 | 0/5 | 1/5 | 1/5 | 1.6 |
| | SD | 0.47 | 0.15 | 1.90 | 0 | 0 | 0 | | | | 3.58 |
| CI + TC0052 (CT681) | X | 5.21 | 5.75 | 4.39 | 1.36 | 1.36 | 0 | 0/5 | 2/5 | 2/5 | 2.2 |
| | SD | 0.38 | 0.26 | 0.60 | 1.56 | 1.35 | 0 | | | | 3.03 |

| | T test | | | |
|---|---|---|---|---|
| | D7 | | D14 | |
| | PBS | CI + GST | PBS | CI + GST |
| 1XPBS | 1 | 0.909339 | 1 | 0.820552 |
| CI + GST | 0.909339 | 1 | 0.820552 | 1 |
| CI + Purified Mopn EB | 0.000761 | 0.001034 | 0.002587 | 0.001136 |
| CI + TC0396 (CT119) | 0.380658 | 0.408431 | 0.383007 | 0.281467 |

Note:
CI = CpG + IFA
X = mean
SD = Standard deviations

TABLE 6
VacExp6 - Balb/c, IM

| | | | | | | Oviduct Pathology | | | |
|---|---|---|---|---|---|---|---|---|---|
| | | | | | | Hydrosalpinx | | | |
| | | IFU | | | | Incidence | | | Severity |
| | | D7 | D14 | D21 | D28 | Uni- | Bi- | Total | Score |
| CI + GST | X | 5.69 | 3.85 | 1.28 | 0.16 | 1/5 | 3/5 | 4/5 | 3.2 |
| | SD | 0.19 | 2.02 | 1.76 | 0.35 | | | | 2.39 |
| CI + | X | 5.26 | 0 | 0 | 0 | 0/5 | 1/5 | 1/5 | 0.6 |
| Purified | SD | 0.18 | 0 | 0 | 0 | | | | 1.34 |
| Mopn EB | | | | | | | | | |
| CI + | X | 5.69 | 3.61 | 0.00 | 0 | 0/5 | 5/5 | 5/5 | 5.6 |
| TC0727 | SD | 0.24 | 2.58 | 0.00 | 0 | | | | 0.89 |
| (CT443) | | | | | | | | | |

| | TTEST | | | |
|---|---|---|---|---|
| | D7 | D14 | D21 | D28 |
| | CI + GST | CI + GST | CI + GST | CI + GST |
| CI + GST | 1 | 1 | 1 | 1 |
| CI + | 0.00625769 | 0.0028 | 0.14316436 | 0.346594 |
| Purified | | | | |
| Mopn EB | | | | |
| CI + | 0.96563701 | 0.87686 | 0.14316436 | 0.346594 |
| TC0727 | | | | |
| (CT443) | | | | |

TABLE 7
VacExp7 - Balb/c, IM

| | | | | | | Oviduct Pathology | | | |
|---|---|---|---|---|---|---|---|---|---|
| | | | | | | Hydrosalpinx | | | |
| | | IFU(Log10) | | | | Incidence | | | Severity |
| | | D7 | D14 | D21 | D28 | Uni- | Bi- | Total | Score |
| CI + GST | X | 5.73 | 5.39 | 0.45 | 0 | 1/5 | 4/5 | 5/5 | 4.20 |
| | SD | 0.18 | 0.43 | 1.00 | 0 | | | | 3.49 |
| CI + Purified Mopn EB | X | 5.03 | 2.83 | 0.32 | 0 | 2/5 | 1/5 | 3/5 | 1.80 |
| | SD | 0.25 | 2.62 | 0.72 | 0 | | | | 2.49 |
| CI + TC0364.2_{(CT089)} | X | 5.83 | 5.75 | 3.18 | 0 | 2/5 | 3/5 | 5/5 | 3.00 |
| | SD | 0.20 | 0.15 | 2.28 | 0 | | | | 2.92 |

Note:
CI = CpG + IFA
X = mean
SD = Standard deviations

TABLE 8
VacExpB - Balb/c, IN

| | | | | | | | | | | Pathology in Oviduct | | | Pathology in Uterine Horn | | |
|---|---|---|---|---|---|---|---|---|---|---|---|---|---|---|---|
| | | IFU(Log10) | | | | | | | | Hydro- | DI | IN | Dila- | DI | IN |
| | | D4 | D7 | D10 | D13 | D16 | D20 | D24 | D27 | salpinx | score | score | tion | score | score |
| CpG | X | 5.69 | 5.37 | 4.91 | 5.33 | 5.03 | 1.81 | 0 | 0 | | 1.8 | 2.6 | | 0 | 1.2 |
| | SD | 0.15 | 0.16 | 0.15 | 0.29 | 0.36 | 1.68 | 0 | 0 | | 1.10 | 0.89 | | 0 | 1.10 |
| LiveMopn(intranasal) | X | 4.14 | 1.0 | 0 | 0 | 0 | 0 | 0 | 0 | | 1 | 1.8 | | 0.2 | 0.2 |
| | SD | 1.19 | 2.23 | 0 | 0 | 0 | 0 | 0 | 0 | | 1.22 | 0.84 | | 0.45 | 0.45 |
| CpG + | X | 6.21 | 5.56 | 4.74 | 4.79 | 4.40 | 0.75 | 0 | 0 | | 2.5 | 2.25 | | 0 | 1 |
| TC0197M(CT812) | SD | 0.11 | 0.17 | 0.18 | 1.30 | 0.37 | 1.5 | 0 | 0 | | 0.58 | 0.96 | | 0 | 0.82 |

Note:
X = mean
SD = Standard deviations
DI score = dilatation score
IN score = inflammation score

TABLE 9
pCT03 in VacExp4 ----- Balb/c, IN

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | Severity |
| | | D3 | D8 | D13 | D16 | D19 | D22 | D25 | D28 | D31 | D34 | Uni- | Bi- | Total | score |
| CpG + GST | X | 5.35 | 5.67 | 5.11 | 3.62 | 2.14 | 1.28 | 0.24 | 0 | 0 | 0 | 1/5 | 4/5 | 5/5 | 5 |
| | SD | 0.79 | 0.26 | 0.15 | 1.45 | 2.12 | 1.29 | 0.53 | 0 | 0 | 0 | | | | 2 |
| CpG + live MoPn EB (50 IFU) | X | 4.07 | 0 | 0 | 0 | 0 | 0 | 0 | 0 | 0 | 0 | 0/5 | 1/5 | 1/5 | 0.4 |
| | SD | 0.40 | 0 | 0 | 0 | 0 | 0 | 0 | 0 | 0 | 0 | | | | 0.89 |
| CpG + UV MoPn EB (1 × 10−6 | X | 5.31 | 5.71 | 3.03 | 3.61 | 2.62 | 0.69 | 0 | 0 | 0 | 0 | 1/5 | 4/5 | 5/5 | 5.2 |
| IFU) | SD | 0.83 | 0.18 | 2.78 | 2.09 | 2.42 | 0.98 | 0 | 0 | 0 | 0 | | | | 3.03 |
| CpG + pMoPn03 (pCT03) | X | 5.52 | 5.60 | 3.05 | 1.83 | 0.44 | 0 | 0 | 0 | 0 | 0 | 1/5 | 1/5 | 2/5 | 1.2 |
| | SD | 0.46 | 0.21 | 2.81 | 2.10 | 0.60 | 0 | 0 | 0 | 0 | 0 | | | | 1.60 |

| | IFU TTEST | | | | |
|---|---|---|---|---|---|
| | D3 | D8 | D13 | D16 | D19 |
| | CpG + GST | CpG + GST | CpG + GST | CpG + GST | CpG + GST |
| CpG + GST | 1 | 1 | 1 | 1 | 1 |
| CpG + live EB | 0.011690131 | 5.18256E−07 | 8.01E−08 | 0.002512362 | 0.043686906 |
| CpG + UV EB | 0.940098097 | 0.790412293 | 0.132088 | 0.99115131 | 0.745547399 |
| CpG + pMoPn03 | 0.685579001 | 0.628116488 | 0.13948 | 0.155121592 | 0.122611205 |

TABLE 10
pCT03 in VacExp7 ----- Balb/c, IM

| | | | | | | Oviduct Pathology | | | |
|---|---|---|---|---|---|---|---|---|---|
| | | | | | | Hydrosalpinx | | | |
| | | IFU(Log10) | | | | Incidence | | | Severity |
| | | D7 | D14 | D21 | D28 | Uni- | Bi- | Total | score |
| CI + GST | X | 5.73 | 5.39 | 0.45 | 0 | 1/5 | 4/5 | 5/5 | 4.20 |
| | SD | 0.18 | 0.43 | 1.00 | 0 | | | | 3.49 |
| CI + | X | 5.03 | 2.83 | 0.32 | 0 | 2/5 | 1/5 | 3/5 | 1.80 |
| Mopn | SD | 0.25 | 2.62 | 0.72 | 0 | | | | 2.49 |
| EB | | | | | | | | | |
| CI + | X | 5.58 | 3.60 | 2.26 | 0 | 0/5 | 5/5 | 5/5 | 4.20 |
| pMoPn03 | SD | 0.18 | 2.10 | 2.25 | 0 | | | | 1.79 |
| (pCT03) | | | | | | | | | |

| | IFU TTEST | | |
|---|---|---|---|
| | D7 | D14 | D21 |
| | CpG + GST | CpG + GST | CpG + GST |
| CI + GST | 1 | 1 | 1 |
| CI + Mopn EB | 0.0009686 | 0.063435448 | 0.823618612 |
| CI + pMoPn03 | 0.2394361 | 0.099443178 | 0.138800272 |

TABLE 11
CT858 in VacExp2 ---Balb/c, IN

| | | | | | | | | | | Oviduct Pathology | | | |
|---|---|---|---|---|---|---|---|---|---|---|---|---|---|
| | | | | | | | | | | Hydrosalpinx | | | |
| | | IFU(Log10) | | | | | | | | Incidence | | | |
| | | D3 | D8 | D13 | D16 | D19 | D22 | D25 | D28 | Uni- | Bi- | Total | Severity |
| CpG | X | 5.69 | 4.90 | 1.74 | 0.96 | 0.38 | 0.14 | 0 | 0 | 2/5 | 1/5 | 3/5 | 2 |
| | SD | 0.48 | 0.61 | 1.99 | 2.15 | 0.85 | 0.31 | 0 | 0 | | | | 2 |
| CpG + UV MoPn EB(1 × 10⁶ IFU) | X | 5.83 | 5.44 | 4.89 | 4.47 | 1.64 | 0 | 0.7171 | 0 | 2/5 | 2/5 | 4/5 | 2.2 |
| | SD | 0.13 | 0.12 | 0.85 | 1.02 | 2.25 | 0 | 1.0561 | 0 | | | | 1.48 |
| CpG + live MoPn(500 IFU) | X | 3.94 | 3 | 0 | 0 | 0 | 0 | 0 | 0 | 0/1 | 0/1 | 0/1 | 0 |
| | SD | 0 | 0 | 0 | 0 | 0 | 0 | 0 | 0 | | | | 0 |
| CpG + CPAF(CT858)-His- | X | 6.09 | 4.74 | 3.80 | 2.261 | 0 | 0 | 0.3253 | 0 | 1/4 | 2/4 | 3/4 | 2.5 |
| WT(Beijing) | SD | 0.18 | 1.32 | 1.59 | 1.152 | 0 | 0 | 0.6505 | 0 | | | | 1.73 |
| CpG + CPAF(CT858-His- | X | 6.19 | 5.30 | 4.60 | 2.47 | 0 | 0 | 0 | 0 | 2/5 | 1/5 | 3/5 | 3 |
| Mut(Beijing) | SD | 0.25 | 0.08 | 0.80 | 1.11 | 0 | 0 | 0 | 0 | | | | 3.32 |

TABLE 12
CT858 in VacExp 5 --- Balb/c, IM

| | | | | | | | | Oviduct Pathology | | | |
|---|---|---|---|---|---|---|---|---|---|---|---|
| | | | | | | | | Hydrosalpinx | | | |
| | | IFU(Log10) | | | | | | Incidence | | | Severity |
| | | D3 | D7 | D14 | D21 | D24 | D27 | Uni- | Bi- | Total | score |
| PBS | X | 5.34 | 5.34 | 3.90 | 0.41 | 0 | 0 | 4/10 | 0/10 | 4/10 | 0.9 |
| | SD | 0.25 | 0.31 | 1.40 | 0.96 | 0 | 0 | | | | 1.20 |
| CI + GST | X | 5.57 | 5.32 | 4.03 | 0.37 | 0.15 | 0 | 2/10 | 2/10 | 4/10 | 1.9 |
| | SD | 0.22 | 0.38 | 1.26 | 0.83 | 0.47 | 0 | | | | 2.51 |
| CI + Purified Mopn EB | X | 5.04 | 3.99 | 1.55 | 0 | 0 | 0 | 2/10 | 0/10 | 2/10 | 0.6 |
| | SD | 0.63 | 1.01 | 1.60 | 0 | 0 | 0 | | | | 1.35 |
| CpG + CPAF(CT858)-His-WT | X | 5.29 | 5.03 | 2.981 | 0 | 0 | 0 | 2/10 | 0/10 | 2/10 | 0.5 |
| | SD | 0.27 | 0.66 | .72 | 0 | 0 | 0 | | | | 1.08 |

CI = CpG + IFA
X = mean
SD = Standard deviations

TABLE 13
CT841, CT101, CT142 and CT806 in VacExp1 ---Balb/c, IM

| | | | | | | | | | | Oviduct Pathology | | | |
|---|---|---|---|---|---|---|---|---|---|---|---|---|---|
| | | | | | | | | | | Hydrosalpinx | | | |
| | | IFU(Log10) | | | | | | | | Incidence | | | |
| | | D5 | D10 | D15 | D18 | D21 | D24 | D27 | D30 | Uni- | Bi- | Total | Severity |
| CI | X | 5.85 | 4.76 | 1.77 | 1.11 | 0 | 0 | 0 | 0 | 1/5 | 2/5 | 3/5 | 1.2 |
| | SD | 0.29 | 0.80 | 2.19 | 1.96 | 0 | 0 | 0 | 0 | | | | 1.10 |
| CI + MoPn EB_{(1 × 10}_{⁵} IFU) | X | 2.54 | 0 | 0.32 | 0 | 0 | 0 | 0 | 0 | 2/5 | 0/5 | 2/5 | 0.4 |
| | SD | 2.23 | 0 | 0.72 | 0 | 0 | 0 | 0 | 0 | | | | 0.55 |
| CI + TC0376_{(CT101)} | X | 5.64 | 4.58 | 2.46 | 2.14 | 0.93 | 0.71 | 0 | 0 | 1/5 | 4/5 | 5/5 | 3 |
| | SD | 0.29 | 0.59 | 1.92 | 1.46 | 1.28 | 1.59 | 0 | 0 | | | | 1.00 |
| CI + TC0190_{(CT806)} | X | 6.18 | 5.07 | 4.92 | 2.30 | 0.85 | 0.51 | 0.50 | 0 | 1/5 | 3/5 | 4/5 | 3.2 |
| | SD | 0.18 | 0.40 | 0.65 | 1.92 | 1.89 | 1.15 | 1.12 | 0 | | | | 2.59 |
| CI + TC0229_{(CT841)} | X | 6.12 | 5.04 | 4.02 | 1.03 | 0 | 0 | 0 | 0 | 0/5 | 5/5 | 5/5 | 6.2 |
| | SD | 0.39 | 0.35 | 1.65 | 1.95 | 0 | 0 | 0 | 0 | | | | 1.30 |
| CI + TC0419_{(CT142)} | X | 5.70 | 4.59 | 4.24 | 1.68 | 0.20 | 0 | 0 | 0 | 1/4 | 3/4 | 4/4 | 3.8 |
| | SD | 0.66 | 0.63 | 0.98 | 1.88 | 0.45 | 0 | 0 | 0 | | | | 0.84 |

Note:
CpG Invivogent
CI = CpG + IFA
X = mean
SD = Standard deviations

TABLE 14
CT143 in VacExp1 --- Balb/c, IM

| | | | | | | | Oviduct Pathology | | | |
|---|---|---|---|---|---|---|---|---|---|---|
| | | | | | | | Hydrosalpinx | | | |
| | | IFU(Log10) | | | | | Incidence | | | Severity |
| | | D5 | D10 | D15 | D18 | D21 | Uni- | Bi- | Total | score |
| CI | X | 5.85 | 4.76 | 1.77 | 1.11 | 0 | 1/5 | 2/5 | 3/5 | 1.2 |
| | SD | 0.29 | 0.80 | 2.19 | 1.96 | 0 | | | | 1.10 |
| CI + MoPn | X | 2.54 | 0 | 0.32 | 0 | 0 | 2/5 | 0/5 | 2/5 | 0.4 |
| | SD | 2.23 | 0 | 0.72 | 0 | 0 | | | | 0.55 |
| CI + TC0420(CT143) | X | 6.11 | 4.16 | 1.14 | 0.30 | 0 | 2/5 | 1/5 | 3/5 | 2 |
| | SD | 0.30 | 1.13 | 2.04 | 0.66 | 0 | | | | 2.55 |

TABLE 15
CT143 in VacExp 3 --- Balb/c, IM

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | |
| | | D3 | D8 | D13 | D16 | D19 | D22 | D25 | D28 | D31 | D34 | Uni- | Bi- | Total | Severity |
| PBS | X | 5.44 | 5.18 | 3.60 | 2.41 | 0.90 | 0.34 | 0 | 0.41 | 0 | 0.28 | 2/5 | 1/5 | 3/5 | 2.8 |
| | SD | 0.35 | 0.33 | 0.61 | 1.75 | 1.23 | 0.76 | 0 | 0.91 | 0 | 0.63 | | | | 2.95 |
| CI + GST | X | 5.82 | 5.03 | 3.73 | 3.38 | 1.82 | 0.93 | 0 | 0 | 0 | 0 | 1/5 | 2/5 | 3/5 | 4 |
| | SD | 0.46 | 0.41 | 1.49 | 2.23 | 2.51 | 0.99 | 0 | 0 | 0 | 0 | | | | 4 |
| CI + MoPn | X | 4.00 | 3.10 | 0 | 0 | 0 | 0 | 0 | 0 | 0 | 0 | 1/5 | 2/5 | 3/5 | 1.4 |
| EB(1 × 10⁵ IFU) | SD | 1.32 | 1.82 | 0 | 0 | 0 | 0 | 0 | 0 | 0 | 0 | | | | 1.67 |
| CI + TC0420(CT143) | X | 5.95 | 5.01 | 4.86 | 4.09 | 2.45 | 0 | 0 | 0 | 0 | 0 | 1/4 | 2/4 | 3/4 | 3.5 |
| | SD | 0.30 | 0.12 | 0.42 | 1.40 | 1.87 | 0 | 0 | 0 | 0 | 0 | | | | 3.42 |

TABLE 16
CT813 in VacExpA --- Balb/c, IN

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | Severity |
| | | D3 | D6 | D9 | D12 | D15 | D18 | D21 | D24 | D27 | D30 | Uni- | Bi- | Total | score |
| M-pTC0199 (CT813) | X | 5.40 | 5.36 | 4.85 | 4.23 | 3.53 | 3.26 | 0.78 | 0 | 0 | 0 | 1/5 | 4/5 | 5/5 | 6.20 |
| | SD | 0.40 | 0.29 | 0.61 | 1.16 | 1.39 | 1.82 | 0.66 | 0 | 0 | 0 | | | | 2.17 |
| pTC0199 (CT813) | X | 6.01 | 5.68 | 4.96 | 4.24 | 3.90 | 3.64 | 2.68 | 0 | 0 | 0 | 1/5 | 4/5 | 5/5 | 5.20 |
| | SD | 0.32 | 0.10 | 0.29 | 0.77 | 1.76 | 2.07 | 1.56 | 0 | 0 | 0 | | | | 2.77 |
| pTC0199/pscmIL12 | X | 6.00 | 5.84 | 4.79 | 4.31 | 3.89 | 3.45 | 1.13 | 0 | 0 | 0 | 0/5 | 5/5 | 5/5 | 4.60 |
| | SD | 0.20 | 0.51 | 0.21 | 0.41 | 0.27 | 1.24 | 0.75 | 0 | 0 | 0 | | | | 2.79 |
| pscmIL12 | X | 5.61 | 5.52 | 4.80 | 2.91 | 1.46 | 1.28 | 0.80 | 0 | 0.08 | 0 | 0/5 | 4/5 | 4/5 | 3.20 |
| | SD | 0.54 | 0.20 | 0.65 | 1.31 | 1.83 | 1.61 | 1.11 | 0 | 0.18 | 0 | | | | 3.03 |
| pcDNA3.1 | X | 5.60 | 5.36 | 4.87 | 3.78 | 3.09 | 1.42 | 1.73 | 1.39 | 0.74 | 0 | 3/5 | 2/5 | 5/5 | 4.20 |
| | SD | 0.50 | 0.16 | 0.28 | 0.92 | 1.32 | 0.32 | 1.12 | 1 | 1.02 | 0 | | | | 2.28 |
| PBS | X | 6.00 | 5.58 | 5.12 | 4.19 | 3.87 | 1.66 | 1.09 | 0 | 0.08 | 0 | 0/5 | 5/5 | 5/5 | 5.80 |
| | SD | 0.25 | 0.27 | 0.30 | 0.30 | 0.59 | 1.49 | 0.23 | 0 | 0.18 | 0 | | | | 2.17 |

TABLE 17
CT240 in VacExp2 --- Balb/c, IM

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | |
| | | D3 | D8 | D13 | D16 | D19 | D22 | D25 | D28 | D31 | D34 | Uni- | Bi- | Total | Severity |
| CpG* | X | 5.69 | 4.90 | 1.74 | 0.96 | 0.38 | 0.14 | 0 | 0 | 0 | 0 | 2/5 | 1/5 | 3/5 | 2 |
| | SD | 0.48 | 0.61 | 1.99 | 2.15 | 0.85 | 0.31 | 0 | 0 | 0 | 0 | | | | 2 |
| CpG + UV MoPn | X | 5.83 | 5.44 | 4.89 | 4.47 | 1.64 | 0 | 0.717 | 0 | 0 | 0 | 2/5 | 2/5 | 4/5 | 2.2 |
| EB(1 × 10⁶ IFU) | SD | 0.13 | 0.12 | 0.85 | 1.02 | 2.25 | 0 | 1.056 | 0 | 0 | 0 | | | | 1.48 |
| CpG + live MoPn | X | 3.94 | 3 | 0 | 0 | 0 | 0 | 0 | 0 | 0 | 0 | 0 | 0 | 0/1 | 0 |
| EB(500 IFU) | SD | 0 | 0 | 0 | 0 | 0 | 0 | 0 | 0 | 0 | 0 | | | | 0 |
| CpG + TC0511(CT240) | X | 5.93 | 5.40 | 3.30 | 0.95 | 0 | 0 | 0 | 0 | 0 | 0 | 1/5 | 4/5 | 5/5 | 4.6 |
| | SD | 0.33 | 0.12 | 2.42 | 2.12 | 0 | 0 | 0 | 0 | 0 | 0 | | | | 1.82 |

Note:
CpG* Invitrogen
X = mean
SD = Standard deviations

TABLE 18
CT240 in VacExp3 --- Balb/c, IN

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | |
| | | D3 | D8 | D13 | D16 | D19 | D22 | D25 | D28 | D31 | D34 | Uni- | Bi- | Total | Severity |
| PBS | X | 5.44 | 5.18 | 3.60 | 2.41 | 0.90 | 0.34 | 0 | 0.41 | 0 | 0.28 | 2/5 | 1/5 | 3/5 | 2.8 |
| | SD | 0.35 | 0.33 | 0.61 | 1.75 | 1.23 | 0.76 | 0 | 0.91 | 0 | 0.63 | | | | 2.95 |
| CI + GST | X | 5.82 | 5.03 | 3.73 | 3.38 | 1.82 | 0.93 | 0 | 0 | 0 | 0 | 1/5 | 2/5 | 3/5 | 4 |
| | SD | 0.46 | 0.41 | 1.49 | 2.23 | 2.51 | 0.99 | 0 | 0 | 0 | 0 | | | | 4 |
| CI + MoPn EB(1 × 10⁵ IFU) | X | 4.00 | 3.10 | 0 | 0 | 0 | 0 | 0 | 0 | 0 | 0 | 1/5 | 2/5 | 3/5 | 1.4 |
| | SD | 1.32 | 1.82 | 0 | 0 | 0 | 0 | 0 | 0 | 0 | 0 | | | | 1.67 |
| CI + TC0511(CT240) | X | 5.83 | 5.25 | 4.49 | 4.09 | 3.00 | 0.90 | 0 | 0 | 0 | 0 | 0 | 4/5 | 4/5 | 4.2 |
| | SD | 0.30 | 0.18 | 1.08 | 1.31 | 2.08 | 1.73 | 0 | 0 | 0 | 0 | | | | 2.77 |

TABLE 19
CT798 and CT067 in VacExp7 ---- Balb/c, IM

| | | | | | | Oviduct Pathology | | | |
|---|---|---|---|---|---|---|---|---|---|
| | | | | | | Hydrosalpinx | | | |
| | | IFU(Log10) | | | | Incidence | | | Severity |
| | | D7 | D14 | D21 | D28 | Uni- | Bi- | Total | score |
| CI + GST | X | 5.73 | 5.39 | 0.45 | 0 | 1/5 | 4/5 | 5/5 | 4.20 |
| | SD | 0.18 | 0.43 | 1.00 | 0 | | | | 3.49 |
| CI + | X | 5.03 | 2.83 | 0.32 | 0 | 2/5 | 1/5 | 3/5 | 1.80 |
| Purified | SD | 0.25 | 2.62 | 0.72 | 0 | | | | 2.49 |
| Mopn EB | | | | | | | | | |
| CI + | X | 5.92 | 5.53 | 4.26 | 0.19 | 1/5 | 4/5 | 5/5 | 3.40 |
| TC0338.1 | SD | 0.15 | 0.22 | 1.80 | 0.43 | | | | 1.14 |
| (CT067) | | | | | | | | | |
| CI + | X | 5.64 | 5.25 | 0.46 | 0 | 1/5 | 4/5 | 5/5 | 5.50 |
| TC0181.4 | SD | 0.23 | 0.38 | 1.03 | 0 | | | | 3.32 |
| (CT798) | | | | | | | | | |

Note:
CI = CpG + IFA
X = mean
SD = Standard deviations

TABLE 20
CT022 in VacExp5 ---- Balb/c, IM

| | | | | | | | | Oviduct Pathology | | | |
|---|---|---|---|---|---|---|---|---|---|---|---|
| | | | | | | | | Hydrosalpinx | | | |
| | | IFU(Log10) | | | | | | Incidence | | | Severity |
| | | D3 | D7 | D14 | D21 | D24 | D27 | Uni- | Bi- | Total | score |
| PBS | X | 5.34 | 5.34 | 3.90 | 0.41 | 0 | 0 | 4/10 | 0/10 | 4/10 | 0.9 |
| | SD | 0.25 | 0.31 | 1.40 | 0.96 | 0 | 0 | | | | 1.20 |
| CI + GST | X | 5.57 | 5.32 | 4.03 | 0.37 | 0.15 | 0 | 2/10 | 2/10 | 4/10 | 1.9 |
| | SD | 0.22 | 0.38 | 1.26 | 0.83 | 0.47 | 0 | | | | 2.51 |
| CI + Purified Mopn EB | X | 5.04 | 3.99 | 1.55 | 0 | 0 | 0 | 2/10 | 0/10 | 2/10 | 0.6 |
| | SD | 0.63 | 1.01 | 1.60 | 0 | 0 | 0 | | | | 1.35 |
| CI + TC291 (CT022) | X | 4.50 | 5.01 | 3.94 | 2.04 | 0 | 0 | 2/5 | 1/5 | 3/5 | 2.4 |
| | SD | 2.02 | 0.89 | 2.21 | 2.05 | 0 | 0 | | | | 2.61 |

CI = CpG + IFA
X = mean
SD = Standard deviations

TABLE 21
CT456 in VacExp2 ---- Balb/c, IN

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | |
| | | D3 | D8 | D13 | D16 | D19 | D22 | D25 | D28 | D31 | D34 | Uni- | Bi- | Total | Severity |
| CpG* | X | 5.69 | 4.90 | 1.74 | 0.96 | 0.38 | 0.14 | 0 | 0 | 0 | 0 | 2/5 | 1/5 | 3/5 | 2 |
| | SD | 0.48 | 0.61 | 1.99 | 2.15 | 0.85 | 0.31 | 0 | 0 | 0 | 0 | | | | 2 |
| CpG + UV MoPn | X | 5.83 | 5.44 | 4.89 | 4.47 | 1.64 | 0 | 0.717 | 0 | 0 | 0 | 2/5 | 2/5 | 4/5 | 2.2 |
| EB(1 × 10⁶ IFU) | SD | 0.13 | 0.12 | 0.85 | 1.02 | 2.25 | 0 | 1.056 | 0 | 0 | 0 | | | | 1.48 |
| CpG + live MoPn(500 IFU) | X | 3.94 | 3 | 0 | 0 | 0 | 0 | 0 | 0 | 0 | 0 | 0 | 0 | 0/1 | 0 |
| | SD | 0 | 0 | 0 | 0 | 0 | 0 | 0 | 0 | 0 | 0 | | | | 0 |
| CpG + TC0741(CT456, Tarp) | X | 5.96 | 5.17 | 5.21 | 4.19 | 3.06 | 1.24 | 0.891 | 0.727 | 0.31 | 0.4 | 0 | 2/5 | 2/5 | 2.2 |
| | SD | 0.57 | 0.43 | 0.44 | 2.36 | 2.09 | 1.80 | 1.629 | 1.626 | 0.7 | 0.9 | | | | 3.19 |

Note:
CI = CpG + IFA
X = mean
SD = Standard deviations

TABLE 22
CT828 in VacExp 2 --- Balb/c, IN

| | | | | | | | | | | | | Oviduct Pathology | | | |
|---|---|---|---|---|---|---|---|---|---|---|---|---|---|---|---|
| | | | | | | | | | | | | Hydrosalpinx | | | |
| | | IFU(Log10) | | | | | | | | | | Incidence | | | |
| | | D3 | D8 | D13 | D16 | D19 | D22 | D25 | D28 | D31 | D34 | Uni- | Bi- | Total | Severity |
| CpG* | X | 5.69 | 4.90 | 1.74 | 0.96 | 0.38 | 0.14 | 0 | 0 | 0 | 0 | 2/5 | 1/5 | 3/5 | 2 |
| | SD | 0.48 | 0.61 | 1.99 | 2.15 | 0.85 | 0.31 | 0 | 0 | 0 | 0 | | | | 2 |
| CpG + UV MoPn | X | 5.83 | 5.44 | 4.89 | 4.47 | 1.64 | 0 | 0.717 | 0 | 0 | 0 | 2/5 | 2/5 | 4/5 | 2.2 |
| EB(1 × 10⁶ IFU) | SD | 0.13 | 0.12 | 0.85 | 1.02 | 2.25 | 0 | 1.056 | 0 | 0 | 0 | | | | 1.48 |
| CpG + live | X | 3.94 | 3 | 0 | 0 | 0 | 0 | 0 | 0 | 0 | 0 | 0 | 0 | 0/1 | 0 |
| MoPn(500 IFU) | SD | 0 | 0 | 0 | 0 | 0 | 0 | 0 | 0 | 0 | 0 | | | | 0 |
| CpG + CT828 | X | 5.11 | 5.12 | 3.58 | 2.06 | 0.79 | 0 | 0 | 0 | 0 | 0 | 1/5 | 2/5 | 3/5 | 1.8 |
| | SD | 1.81 | 0.54 | 2.18 | 1.95 | 1.14 | 0 | 0 | 0 | 0 | 0 | | | | 2.05 |

Note:
X = mean
SD = Standard deviations

TABLE 23
CT828 in VacExp 7 --- Balb/c, IM

| | | | | | | Oviduct Pathology | | | |
|---|---|---|---|---|---|---|---|---|---|
| | | | | | | Hydrosalpinx | | | |
| | | IFU(Log10) | | | | Incidence | | | Severity |
| | | D7 | D14 | D21 | D28 | Uni- | Bi- | Total | score |
| CI + GST | X | 5.73 | 5.39 | 0.45 | 0 | 1/5 | 4/5 | 5/5 | 4.20 |
| | SD | 0.18 | 0.43 | 1.00 | 0 | | | | 3.49 |
| CI + | X | 5.03 | 2.83 | 0.32 | 0 | 2/5 | 1/5 | 3/5 | 1.80 |
| Purified | SD | 0.25 | 2.62 | 0.72 | 0 | | | | 2.49 |
| Mopn EB | | | | | | | | | |
| CI + | X | 5.73 | 4.30 | 2.23 | 0.16 | 0/5 | 0/5 | 0/5 | 0.00 |
| TC0215.3 | SD | 0.22 | 2.42 | 1.92 | 0.35 | | | | 0.00 |
| (CT828) | | | | | | | | | |

CI = CpG + IFA
X = mean
SD = Standard deviations

TABLE 24
CT381 in VacExp6 ---- Balb/c, IM

| | | | | | | Pathology in Oviduct | | |
|---|---|---|---|---|---|---|---|---|
| | | D7 | D14 | D21 | D28 | Hydrosalpinx * | | |
| | | IFU | | | | left side | right side | score |
| CI + GST | BB1L | 5.7125 | 1.6812 | 0 | 0 | 1 | 1 | 2 |
| | BB1R | 5.4284 | 5.7761 | 3.4636 | 0 | 0 | 0 | 0 |
| | BB1LR | 5.6725 | 1.6435 | 0 | 0 | 0 | 3 | 3 |
| | BB1RR | 5.977 | 4.8023 | 2.9243 | 0.77815 | 3 | 3 | 6 |
| | BB1N | 5.6713 | 5.3276 | 0 | 0 | 2 | 3 | 5 |
| | x | 5.6923 | 3.8461 | 1.2776 | 0.15563 | incidence: 4/5 | | 3.2 |
| | SD | 0.1949 | 2.0231 | 1.7598 | 0.348 | | | 2.387467277 |
| CI + Purified Mopn EB | BB2L | 5.3636 | 0 | 0 | 0 | 1 | 2 | 3 |
| | BB2R | 5.2401 | 0 | 0 | 0 | 0 | 0 | 0 |
| | BB2LR | 4.9514 | 0 | 0 | 0 | 0 | 0 | 0 |
| | BB2RR | 5.3692 | 0 | 0 | 0 | 0 | 0 | 0 |
| | BB2N | 5.3612 | 0 | 0 | 0 | 0 | 0 | 0 |
| | X | 5.2571 | 0 | 0 | 0 | incidence: 1/5 | | 0.6 |
| | SD | 0.1792 | 0 | 0 | 0 | | | 1.341640786 |
| CI + TC0660 (CT381) | BB9L | 6.2243 | 3.8561 | 4.7176 | 0 | 0 | 3 | 3 |
| | BB9R | 5.6949 | 3.9659 | 0 | 0 | 0 | 0 | 0 |
| | BB9LR | 5.7407 | 0 | 0 | 0 | 1 | 0 | 1 |
| | BB9RR | 5.7866 | 5.8964 | 0 | 0 | 4 | 4 | 8 |
| | BB9N | 5.6007 | 5.4764 | 0 | 0 | 1 | 4 | 5 |
| | X | 5.8094 | 3.839 | 0.9435 | 0 | incidence: 4/5 | | 3.4 |
| | SD | 0.2419 | 2.3275 | 2.1098 | 0 | | | 3.209361307 |

| | | | |
|---|---|---|---|
| D7 | D14 | D21 | D28 |

Average IFU

| | | | | |
|---|---|---|---|---|
| CI + GST | 5.69233769 | 3.8461 | 1.2776 | 0.1556 |
| CI + Purified MopnEB | 5.25707851 | 0 | 0 | 0 |
| CI + TC0660 (CT381) | 5.80944414 | 3.839 | 0.9435 | 0 |

SD

| | | | | |
|---|---|---|---|---|
| CI + GST | 0.19491034 | 2.0231 | 1.7598 | 0.348 |
| CI + Purified MopnEB | 0.17922138 | 0 | 0 | 0 |
| CI + TC0660 (CT381) | 0.24190396 | 2.3275 | 2.1098 | 0 |

1. A composition comprising one or more than one isolated Chlamydia trachomatis protein selected from the group consisting of (1) pCT03, (2) CT858, (3) CT841, (4) CT443, (5) CT143, (6) CT101, (7) CT694, (8) CT813, (9) CT142, (10) CT089, (11) CT442, (12) CT529, (13) CT806, (14) CT147, (15) CT119, (16) CT240, (17) CT812, (18) CT798, (19) CT067, (20) CT695, (21) CT875, (22) CT681, (23) CT795, (24) CT022, (25) CT456, (26) CT828, (27) CT381, an immunogenic fragment thereof, a homologue thereof from a different Chlamydia species and any combination thereof.

2. The composition of claim 1, wherein the one or more than one isolated Chlamydia trachomatis protein is selected from the group consisting of (1) pCT03, (2) CT858, (4) CT443, (7), CT694, (10) CT089, (11) CT 442, (12) CT529, (14) CT147, (15) CT119, (17) CT812, (20) CT695, (22) CT681, (23) CT795, an immunogenic fragment thereof, a homologue thereof from a different Chlamydia species and any combination thereof.

3. The composition of claim 1, further comprising an isolated Chlamydia trachomatis protein selected from the group consisting of porin B (PorB), CT110 (HSP90), CT858 (CPAF), CT681 (MOMP), an immunogenic fragment thereof, a homologue thereof from a different Chlamydia species and any combination thereof.

4. A pharmaceutical composition comprising the composition of claim 1 and a pharmaceutically acceptable carrier.

5. The composition of claim 1, further comprising an adjuvant and/or an immunostimulant.

6. The composition of claim 5, wherein the adjuvant and/or immunostimulant is selected from the group consisting of CpG, IL-12 and any combination thereof.

7. The composition of claim 1, further comprising a protein or immunogenic fragment thereof of a pathogenic organism other than Chlamydia trachomatis.

8. The composition of claim 7, wherein the pathogenic organism is selected from the group consisting of Chlamydia muridarum, Chlamydia pneumoniae, Chlamydia caviae. Trichomonas vaginalis, Candida albicans, Neisseria gonorrheae, Treponema pallidum, Herpes simplex virus, human papilloma virus and human immunodeficiency virus.

9. A method of eliciting an immune response against Chlamydia in a subject, comprising administering to the subject an effective amount of the composition of claim 1, thereby eliciting an immune response against Chlamydia in the subject.

10. A method of inducing immunity against Chlamydia in a subject, comprising administering to the subject an amount of a composition of claim 1 sufficient to elicit an immune response, wherein said immune response is sufficient to decrease risk of onset of disease caused by Chlamydia.

11. A method of treating an infection by Chlamydia in a subject, comprising administering to the subject an effective amount of the composition of claim 1, thereby treating an infection by Chlamydia in the subject.

12. A method of preventing disease caused by infection by Chlamydia in a subject, comprising administering to the subject an effective amount of the composition of claim 1, thereby preventing infection by Chlamydia in the subject.

13. A method of reducing the likelihood of infertility due to Chlamydia infection in a subject, comprising administering to the subject an effective amount of the composition of claim 1, thereby reducing the likelihood of infertility due to Chlamydia infection in the subject.

14. A method of reducing the incidence of hydrosalpinx due to Chlamydia infection in a subject, comprising administering to the subject an effective amount of the composition of claim 1, thereby reducing the incidence of hydrosalpinx due to Chlamydia infection in the subject.

15. A method of detecting an antibody to Chlamydia in a sample, comprising:
a) contacting the sample with the composition of claim 1 under conditions whereby an antigen/antibody complex can form; and
b) detecting antigen/antibody complex formation, thereby detecting an antibody to Chlamydia in the sample.

16. A method of diagnosing a Chlamydia infection in a subject, comprising:
a) contacting a sample from the subject with the composition of claim 1 under conditions whereby an antigen/antibody complex can form; and
b) detecting antigen/antibody complex formation, thereby diagnosing a Chlamydia infection in the subject.

17. A method of detecting a Chlamydia protein in a sample, comprising:
a) contacting the sample with an antibody that specifically binds a Chlamydia protein or immunogenic fragment thereof of claim 1 under conditions whereby an antigen/antibody complex can form; and
b) detecting antigen/antibody complex formation, thereby detecting a Chlamydia protein in the sample.

18. A method of diagnosing a Chlamydia infection in a subject, comprising:
a) contacting a sample from the subject with an antibody that specifically binds a Chlamydia protein or immunogenic fragment thereof of claim 1 under conditions whereby an antigen/antibody complex can form; and
b) detecting antigen/antibody complex formation, thereby diagnosing a Chlamydia infection in the subject.

19. The method of claim 15, wherein the sample is selected from the group consisting of vaginal fluid, vaginal tissue, vaginal washing, vaginal swab, urethral swab, urine, blood, serum, plasma, saliva, semen, urethral discharge, vaginal discharge and any combination thereof.

20. The method of claim 9, wherein the subject is human.
